SEQUENCE LISTING (109 sequences)

SEQ ID NO: 1 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacatgggcgatgactgacatgttccgaccctatagtgagtcgtattaccggcc

SEQ ID NO: 2 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatatcatccttgcgagtgtcagtgaagcaccctatagtgagtcgtattaccggcc

SEQ ID NO: 3 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagaggaagcccgagccatccttgctcaccctatagtgagtcgtattaccggcc

SEQ ID NO: 4 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagctgtcacctctgaagagaagtccaaccctatagtgagtcgtattaccggcc

SEQ ID NO: 5 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacacagtttctgtctgcacacacacgaccctatagtgagtcgtattaccggcc

SEQ ID NO: 6 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgataaggtctggtagagctcctcgggctcaccctatagtgagtcgtattaccggcc

SEQ ID NO: 7 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagccctcgccgtaggtagagatgatgaccctatagtgagtcgtattaccggcc

SEQ ID NO: 8 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatatctccgcaaactgcttcagggtgaaaccctatagtgagtcgtattaccggcc

SEQ ID NO: 9 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatactcggctgcgctcagctggccctcaaccctatagtgagtcgtattaccggcc

SEQ ID NO: 10 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatactgaagttcatgatggcgtcgaacaaccctatagtgagtcgtattaccggcc

SEQ ID NO: 11 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacgctgtccagcttgatgtccagcttaccctatagtgagtcgtattaccggcc

SEQ ID NO: 12 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgataacggtacttctgtgcagtgacggggaccctatagtgagtcgtattaccggcc

SEQ ID NO: 13 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgataaagcggcctttgtcagaggttggcaaccctatagtgagtcgtattaccggcc

SEQ ID NO: 14 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacgtagcggcccatcatcagaatggtaccctatagtgagtcgtattaccggcc

SEQ ID NO: 15 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacactccgaccagccccacaatgtttaccctatagtgagtcgtattaccggcc

SEQ ID NO: 16 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatattgtgagcgtgctcaaaggtagtgaaccctatagtgagtcgtattaccggcc

SEQ ID NO: 17 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatatgttctccagactcctcaatgatgcaccctatagtgagtcgtattaccggcc

SEQ ID NO: 18 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgataccaggtccttaaggcagatctccagaccctatagtgagtcgtattaccggcc

SEQ ID NO: 19 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagtacagccggttgtgcttattggggaccctatagtgagtcgtattaccggcc

SEQ ID NO: 20 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagccatcagggccaaagcaccagatcaccctatagtgagtcgtattaccggcc

SEQ ID NO: 21 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatatcattcaggtactgcacacccttggaccctatagtgagtcgtattaccggcc

SEQ ID NO: 22 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatacgcatgttttcctcacagagagcgcaccctatagtgagtcgtattaccggcc

SEQ ID NO: 23 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgataatgccacccaccacttgctcaggacaccctatagtgagtcgtattaccggcc

SEQ ID NO: 24 - DNA, 166 bp, synthetic construct
  source 1..166: mol_type = other DNA
gccccatcatgtgcctttcctttgacgaagcctgacaatgacgcgacatagattgacgaagcctgacgtacaacctcggttaattgacgaagcctgacagctcggcctgtgatagccagtggtcaaacacgcactggggaccctatagtgagtcgtattaccggcc

SEQ ID NO: 25 - DNA, 166 bp, synthetic construct
  source 1..166: mol_type = other DNA
gccccatcatgtgcctttcctggtcagcgtggacttgccgtggtccattgacgaagcctgacccgcaagtaatctgtattgacgaagcctgaccgtcgataggactcaattgacgaagcctgacctggtgataacgctaaccctatagtgagtcgtattaccggcc

SEQ ID NO: 26 - DNA, 166 bp, synthetic construct
  source 1..166: mol_type = other DNA
gccccatcatgtgcctttcctatttgattgtgatgcagcgctcctgattgacgaagcctgacccgcaagtaatctgtattgacgaagcctgaccgtcgataggactcaattgacgaagcctgacctggtgataacgctaaccctatagtgagtcgtattaccggcc

SEQ ID NO: 27 - DNA, 166 bp, synthetic construct
  source 1..166: mol_type = other DNA
gccccatcatgtgcctttccttggcctggagagtcgatgaggttgaattgacgaagcctgacccgcaagtaatctgtattgacgaagcctgaccgtcgataggactcaattgacgaagcctgacctggtgataacgctaaccctatagtgagtcgtattaccggcc

SEQ ID NO: 28 - DNA, 166 bp, synthetic construct
  source 1..166: mol_type = other DNA
gccccatcatgtgcctttcctgagctccatcggtgacacgcaaggcattgacgaagcctgacccgcaagtaatctgtattgacgaagcctgaccgtcgataggactcaattgacgaagcctgacctggtgataacgctaaccctatagtgagtcgtattaccggcc

SEQ ID NO: 29 - DNA, 166 bp, synthetic construct
  source 1..166: mol_type = other DNA
gccccatcatgtgcctttcctatgcgttcagcgatggcctggcgcaattgacgaagcctgacccgcaagtaatctgtattgacgaagcctgaccgtcgataggactcaattgacgaagcctgacctggtgataacgctaaccctatagtgagtcgtattaccggcc

SEQ ID NO: 30 - DNA, 166 bp, synthetic construct
  source 1..166: mol_type = other DNA
gccccatcatgtgcctttcctgttgacgttctccacgatgcgctggattgacgaagcctgacccgcaagtaatctgtattgacgaagcctgaccgtcgataggactcaattgacgaagcctgacctggtgataacgctaaccctatagtgagtcgtattaccggcc

SEQ ID NO: 31 - DNA, 166 bp, synthetic construct
  source 1..166: mol_type = other DNA
gccccatcatgtgcctttcctatgatattgcccatgggcccactctattgacgaagcctgacccgcaagtaatctgtattgacgaagcctgaccgtcgataggactcaattgacgaagcctgacctggtgataacgctaaccctatagtgagtcgtattaccggcc

SEQ ID NO: 32 - DNA, 166 bp, synthetic construct
  source 1..166: mol_type = other DNA
gccccatcatgtgcctttcctcttggctgcaaacttggccacatacattgacgaagcctgacccgcaagtaatctgtattgacgaagcctgaccgtcgataggactcaattgacgaagcctgacctggtgataacgctaaccctatagtgagtcgtattaccggcc

SEQ ID NO: 33 - DNA, 166 bp, synthetic construct
  source 1..166: mol_type = other DNA
gccccatcatgtgcctttcctatcatgtcctctactttcttggcacattgacgaagcctgacccgcaagtaatctgtattgacgaagcctgaccgtcgataggactcaattgacgaagcctgacctggtgataacgctaaccctatagtgagtcgtattaccggcc

SEQ ID NO: 34 - DNA, 166 bp, synthetic construct
  source 1..166: mol_type = other DNA
gccccatcatgtgcctttcctcgatcagcttggctgtctcctccttattgacgaagcctgacccgcaagtaatctgtattgacgaagcctgaccgtcgataggactcaattgacgaagcctgacctggtgataacgctaaccctatagtgagtcgtattaccggcc

SEQ ID NO: 35 - DNA, 166 bp, synthetic construct
  source 1..166: mol_type = other DNA
gccccatcatgtgcctttccttggcttgccctccttgtctttgtccattgacgaagcctgacccgcaagtaatctgtattgacgaagcctgaccgtcgataggactcaattgacgaagcctgacctggtgataacgctaaccctatagtgagtcgtattaccggcc

SEQ ID NO: 36 - DNA, 166 bp, synthetic construct
  source 1..166: mol_type = other DNA
gccccatcatgtgcctttcctggaggcccctcgtatagcagctcacattgacgaagcctgacccgcaagtaatctgtattgacgaagcctgaccgtcgataggactcaattgacgaagcctgacctggtgataacgctaaccctatagtgagtcgtattaccggcc

SEQ ID NO: 37 - DNA, 166 bp, synthetic construct
  source 1..166: mol_type = other DNA
gccccatcatgtgcctttcctcagagaacactctaccaaaggcataattgacgaagcctgacccgcaagtaatctgtattgacgaagcctgaccgtcgataggactcaattgacgaagcctgacctggtgataacgctaaccctatagtgagtcgtattaccggcc

SEQ ID NO: 38 - DNA, 166 bp, synthetic construct
  source 1..166: mol_type = other DNA
gccccatcatgtgcctttcctacatggcacgtcctcaatcggctccattgacgaagcctgacccgcaagtaatctgtattgacgaagcctgaccgtcgataggactcaattgacgaagcctgacctggtgataacgctaaccctatagtgagtcgtattaccggcc

SEQ ID NO: 39 - DNA, 166 bp, synthetic construct
  source 1..166: mol_type = other DNA
gccccatcatgtgcctttcctgtccccgtcttcacaaggaactggtattgacgaagcctgacccgcaagtaatctgtattgacgaagcctgaccgtcgataggactcaattgacgaagcctgacctggtgataacgctaaccctatagtgagtcgtattaccggcc

SEQ ID NO: 40 - DNA, 166 bp, synthetic construct
  source 1..166: mol_type = other DNA
gccccatcatgtgcctttccttgacgctgaacttcatcacgcgcatattgacgaagcctgacccgcaagtaatctgtattgacgaagcctgaccgtcgataggactcaattgacgaagcctgacctggtgataacgctaaccctatagtgagtcgtattaccggcc

SEQ ID NO: 41 - DNA, 166 bp, synthetic construct
  source 1..166: mol_type = other DNA
gccccatcatgtgcctttcctgcagctcaccggcgccagcaataatattgacgaagcctgacccgcaagtaatctgtattgacgaagcctgaccgtcgataggactcaattgacgaagcctgacctggtgataacgctaaccctatagtgagtcgtattaccggcc

SEQ ID NO: 42 - DNA, 166 bp, synthetic construct
  source 1..166: mol_type = other DNA
gccccatcatgtgcctttcctgatggggatgcaggcatggtcctccattgacgaagcctgacccgcaagtaatctgtattgacgaagcctgaccgtcgataggactcaattgacgaagcctgacctggtgataacgctaaccctatagtgagtcgtattaccggcc

SEQ ID NO: 43 - DNA, 166 bp, synthetic construct
  source 1..166: mol_type = other DNA
gccccatcatgtgcctttcctccatcagggaagggcctggccttcaattgacgaagcctgacccgcaagtaatctgtattgacgaagcctgaccgtcgataggactcaattgacgaagcctgacctggtgataacgctaaccctatagtgagtcgtattaccggcc

SEQ ID NO: 44 - DNA, 166 bp, synthetic construct
  source 1..166: mol_type = other DNA
gccccatcatgtgcctttcctatgtcggtgagaatgttggggccagattgacgaagcctgacccgcaagtaatctgtattgacgaagcctgaccgtcgataggactcaattgacgaagcctgacctggtgataacgctaaccctatagtgagtcgtattaccggcc

SEQ ID NO: 45 - DNA, 166 bp, synthetic construct
  source 1..166: mol_type = other DNA
gccccatcatgtgcctttcctagccagccaccacactgtccttgatattgacgaagcctgacccgcaagtaatctgtattgacgaagcctgaccgtcgataggactcaattgacgaagcctgacctggtgataacgctaaccctatagtgagtcgtattaccggcc

SEQ ID NO: 46 - DNA, 166 bp, synthetic construct
  source 1..166: mol_type = other DNA
gccccatcatgtgcctttcctcatcatgaacatcaaaccgcacaccattgacgaagcctgacccgcaagtaatctgtattgacgaagcctgaccgtcgataggactcaattgacgaagcctgacctggtgataacgctaaccctatagtgagtcgtattaccggcc

SEQ ID NO: 47 - DNA, 166 bp, synthetic construct
  source 1..166: mol_type = other DNA
gccccatcatgtgcctttcctcacgcttcctgttcaggacaccgtaattgacgaagcctgacccgcaagtaatctgtattgacgaagcctgaccgtcgataggactcaattgacgaagcctgacctggtgataacgctaaccctatagtgagtcgtattaccggcc

SEQ ID NO: 48 - DNA, 166 bp, synthetic construct
  source 1..166: mol_type = other DNA
gccccatcatgtgcctttcctgtcaaaaggatccccaggcaggatcattgacgaagcctgacccgcaagtaatctgtattgacgaagcctgaccgtcgataggactcaattgacgaagcctgacctggtgataacgctaaccctatagtgagtcgtattaccggcc

SEQ ID NO: 49 - DNA, 123 bp, synthetic construct
  source 1..123: mol_type = other DNA
gccccatcatgtgcctttccttacccgttgagtggcagggtatctcgcgtctacccgttgagtggcagggtatctcgcgtcctatgtcgcgtcattaccctatagtgagtcgtattaccggcc

SEQ ID NO: 50 - DNA, 123 bp, synthetic construct
  source 1..123: mol_type = other DNA
gccccatcatgtgcctttccttacccgttgagtggcagattagcgccgtattacccgttgagtggcagattagcgccgtatatcacaggccgagctaccctatagtgagtcgtattaccggcc

SEQ ID NO: 51 - DNA, 123 bp, synthetic construct
  source 1..123: mol_type = other DNA
gccccatcatgtgcctttccttacccgttgagtggcaagagtaccgtcgattacccgttgagtggcaagagtaccgtcgattagcgttatcaccagaccctatagtgagtcgtattaccggcc

SEQ ID NO: 52 - DNA, 123 bp, synthetic construct
  source 1..123: mol_type = other DNA
gccccatcatgtgcctttccttacccgttgagtggccctagactgatcgcgtacccgttgagtggccctagactgatcgcgacagattacttgcggaccctatagtgagtcgtattaccggcc

SEQ ID NO: 53 - DNA, 123 bp, synthetic construct
  source 1..123: mol_type = other DNA
gccccatcatgtgcctttccttacccgttgagtggctggcctaatcgcgaatacccgttgagtggctggcctaatcgcgaataaccgaggttgtacaccctatagtgagtcgtattaccggcc

SEQ ID NO: 54 - DNA, 123 bp, synthetic construct
  source 1..123: mol_type = other DNA
gccccatcatgtgcctttccttacccgttgagtggccaatcttcgcgagtatacccgttgagtggccaatcttcgcgagtatgagtcctatcgacgaccctatagtgagtcgtattaccggcc

SEQ ID NO: 55 - DNA, 123 bp, synthetic construct
  source 1..123: mol_type = other DNA
gccccatcatgtgcctttccttacccgttgagtggcaatgacgcgacatagtacccgttgagtggcaatgacgcgacataggacgcgagataccctaccctatagtgagtcgtattaccggcc

SEQ ID NO: 56 - DNA, 123 bp, synthetic construct
  source 1..123: mol_type = other DNA
gccccatcatgtgcctttccttacccgttgagtggcagctcggcctgtgattacccgttgagtggcagctcggcctgtgatatacggcgctaatctaccctatagtgagtcgtattaccggcc

SEQ ID NO: 57 - DNA, 123 bp, synthetic construct
  source 1..123: mol_type = other DNA
gccccatcatgtgcctttccttacccgttgagtggcctggtgataacgctatacccgttgagtggcctggtgataacgctaatcgacggtactcttaccctatagtgagtcgtattaccggcc

SEQ ID NO: 58 - DNA, 123 bp, synthetic construct
  source 1..123: mol_type = other DNA
gccccatcatgtgcctttccttacccgttgagtggcccgcaagtaatctgttacccgttgagtggcccgcaagtaatctgtcgcgatcagtctaggaccctatagtgagtcgtattaccggcc

SEQ ID NO: 59 - DNA, 123 bp, synthetic construct
  source 1..123: mol_type = other DNA
gccccatcatgtgcctttccttacccgttgagtggcgtacaacctcggttatacccgttgagtggcgtacaacctcggttattcgcgattaggccaaccctatagtgagtcgtattaccggcc

SEQ ID NO: 60 - DNA, 123 bp, synthetic construct
  source 1..123: mol_type = other DNA
gccccatcatgtgcctttccttacccgttgagtggccgtcgataggactcatacccgttgagtggccgtcgataggactcatactcgcgaagattgaccctatagtgagtcgtattaccggcc

SEQ ID NO: 61 - DNA, 144 bp, synthetic construct
  source 1..144: mol_type = other DNA
gccccatcatgtgcctttccttgacgaagcctgacggtctcggatatgcaaatgatggcgcggatctggtctaccgtgaagttcacattgacgaagcctgactgcgtacgacaatgtaccctatagtgagtcgtattaccggcc

SEQ ID NO: 62 - DNA, 128 bp, synthetic construct
  source 1..128: mol_type = other DNA
gccccatcatgtgcctttcctacccgttgagtggcaaataggttcccgttcgatacccgttgagtggcaaataggttcccgttcgatggcgagaattcatgaccctatagtgagtcgtattaccggcc

SEQ ID NO: 63 - DNA, 128 bp, synthetic construct
  source 1..128: mol_type = other DNA
gccccatcatgtgcctttcctacccgttgagtggcaacctttagacgccgtaatacccgttgagtggcaacctttagacgccgtaacgcgaagatgcaattaccctatagtgagtcgtattaccggcc

SEQ ID NO: 64 - DNA, 128 bp, synthetic construct
  source 1..128: mol_type = other DNA
gccccatcatgtgcctttcctacccgttgagtggcaaagattagcgccgtatatacccgttgagtggcaaagattagcgccgtataatcacaggccgagctaccctatagtgagtcgtattaccggcc

SEQ ID NO: 65 - DNA, 128 bp, synthetic construct
  source 1..128: mol_type = other DNA
gccccatcatgtgcctttcctacccgttgagtggcaaactagtcgaccgtccatacccgttgagtggcaaactagtcgaccgtccaataccggccttgagtaccctatagtgagtcgtattaccggcc

SEQ ID NO: 66 - DNA, 128 bp, synthetic construct
  source 1..128: mol_type = other DNA
gccccatcatgtgcctttcctacccgttgagtggcaaataccttccgcgtatatacccgttgagtggcaaataccttccgcgtataaacgctgtacgagtgaccctatagtgagtcgtattaccggcc

SEQ ID NO: 67 - DNA, 128 bp, synthetic construct
  source 1..128: mol_type = other DNA
gccccatcatgtgcctttcctacccgttgagtggcaacggctatactcgtacatacccgttgagtggcaacggctatactcgtacaacagttcgcgtatcaaccctatagtgagtcgtattaccggcc

SEQ ID NO: 68 - DNA, 128 bp, synthetic construct
  source 1..128: mol_type = other DNA
gccccatcatgtgcctttcctacccgttgagtggcaaggtttagacgtcgacatacccgttgagtggcaaggtttagacgtcgacatgcatatccgagaccaccctatagtgagtcgtattaccggcc

SEQ ID NO: 69 - DNA, 128 bp, synthetic construct
  source 1..128: mol_type = other DNA
gccccatcatgtgcctttcctacccgttgagtggcaacgagattagcgcggtatacccgttgagtggcaacgagattagcgcggtaacattgtcgtacgcaaccctatagtgagtcgtattaccggcc

SEQ ID NO: 70 - DNA, 128 bp, synthetic construct
  source 1..128: mol_type = other DNA
gccccatcatgtgcctttccttgacgaagcctgacaacatgaattctcgccaattgacgaagcctgacaacatgaattctcgccaacgaacgggaacctataccctatagtgagtcgtattaccggcc

SEQ ID NO: 71 - DNA, 128 bp, synthetic construct
  source 1..128: mol_type = other DNA
gccccatcatgtgcctttccttgacgaagcctgacaaaattgcatcttcgcgattgacgaagcctgacaaaattgcatcttcgcgatacggcgtctaaaggaccctatagtgagtcgtattaccggcc

SEQ ID NO: 72 - DNA, 128 bp, synthetic construct
  source 1..128: mol_type = other DNA
gccccatcatgtgcctttccttgacgaagcctgacaaagctcggcctgtgatattgacgaagcctgacaaagctcggcctgtgataatacggcgctaatctaccctatagtgagtcgtattaccggcc

SEQ ID NO: 73 - DNA, 128 bp, synthetic construct
  source 1..128: mol_type = other DNA
gccccatcatgtgcctttccttgacgaagcctgacaaactcaaggccggtatattgacgaagcctgacaaactcaaggccggtataggacggtcgactagtaccctatagtgagtcgtattaccggcc

SEQ ID NO: 74 - DNA, 128 bp, synthetic construct
  source 1..128: mol_type = other DNA
gccccatcatgtgcctttccttgacgaagcctgacaacactcgtacagcgttattgacgaagcctgacaacactcgtacagcgttaatacgcggaaggtataccctatagtgagtcgtattaccggcc

SEQ ID NO: 75 - DNA, 128 bp, synthetic construct
  source 1..128: mol_type = other DNA
gccccatcatgtgcctttccttgacgaagcctgacaatgatacgcgaactgtattgacgaagcctgacaatgatacgcgaactgtagtacgagtatagccgaccctatagtgagtcgtattaccggcc

SEQ ID NO: 76 - DNA, 128 bp, synthetic construct
  source 1..128: mol_type = other DNA
gccccatcatgtgcctttccttgacgaagcctgacaaggtctcggatatgcaattgacgaagcctgacaaggtctcggatatgcaagtcgacgtctaaaccaccctatagtgagtcgtattaccggcc

SEQ ID NO: 77 - DNA, 128 bp, synthetic construct
  source 1..128: mol_type = other DNA
gccccatcatgtgcctttccttgacgaagcctgacaatgcgtacgacaatgtattgacgaagcctgacaatgcgtacgacaatgtaaccgcgctaatctcgaccctatagtgagtcgtattaccggcc

SEQ ID NO: 78 - DNA, 130 bp, synthetic construct
  source 1..130: mol_type = other DNA
gccccatcatgtgcctttccttgacgaagcctgacagctcggcctgtgatattgacgaagcctgacccgcaagtaatctgtaggtcagcgtggacttgccgtgaccctatagtgagtcgtattaccggcc

SEQ ID NO: 79 - DNA, 130 bp, synthetic construct
  source 1..130: mol_type = other DNA
gccccatcatgtgcctttccgtccacatgggcgatgactgaattgacgaagcctgacgtacaacctcggttaattgacgaagcctgaccgtcgataggactcaaccctatagtgagtcgtattaccggcc

SEQ ID NO: 80 - DNA, 144 bp, synthetic construct; identical to SEQ ID NO: 61
  source 1..144: mol_type = other DNA

SEQ ID NO: 81 - DNA, 144 bp, synthetic construct
  source 1..144: mol_type = other DNA
gccccatcatgtgcctttccttgacgaagcctgacggtctcggatatgcaaagtctgtcagcgtggacttgccatggtccacgtggattgacgaagcctgactgcgtacgacaatgtaccctatagtgagtcgtattaccggcc

SEQ ID NO: 82 - DNA, 144 bp, synthetic construct
  source 1..144: mol_type = other DNA
gccccatcatgtgcctttccttgacgaagcctgacggtctcggatatgcaacattctccgagagctcgtagaagagggagatggcaattgacgaagcctgactgcgtacgacaatgtaccctatagtgagtcgtattaccggcc

SEQ ID NO: 83 - DNA, 144 bp, synthetic construct
  source 1..144: mol_type = other DNA
gccccatcatgtgcctttccttgacgaagcctgacggtctcggatatgcaaccggcaccgtccttgctctgcttgatgaagttcaaattgacgaagcctgactgcgtacgacaatgtaccctatagtgagtcgtattaccggcc

SEQ ID NO: 84 - DNA, 144 bp, synthetic construct
  source 1..144: mol_type = other DNA
gccccatcatgtgcctttccttgacgaagcctgacggtctcggatatgcaatcatcatcagcacaggcttgatgcgctcggcaatgattgacgaagcctgactgcgtacgacaatgtaccctatagtgagtcgtattaccggcc

SEQ ID NO: 85 - DNA, 144 bp, synthetic construct
  source 1..144: mol_type = other DNA
gccccatcatgtgcctttccttgacgaagcctgacggtctcggatatgcaacacgatgcgctggaaagtctggtagagctcctcggattgacgaagcctgactgcgtacgacaatgtaccctatagtgagtcgtattaccggcc

SEQ ID NO: 86 - DNA, 144 bp, synthetic construct
  source 1..144: mol_type = other DNA
gccccatcatgtgcctttccttgacgaagcctgacggtctcggatatgcaacctcgccgtaggtggagatgatgacgttcacgttcattgacgaagcctgactgcgtacgacaatgtaccctatagtgagtcgtattaccggcc

SEQ ID NO: 87 - DNA, 144 bp, synthetic construct
  source 1..144: mol_type = other DNA
gccccatcatgtgcctttccttgacgaagcctgacggtctcggatatgcaagacccaaagcccacggtaccgaggacaggatcgatattgacgaagcctgactgcgtacgacaatgtaccctatagtgagtcgtattaccggcc

SEQ ID NO: 88 - DNA, 144 bp, synthetic construct
  source 1..144: mol_type = other DNA
gccccatcatgtgcctttccttgacgaagcctgacggtctcggatatgcaaacttgctgaacttgccgttggctgggtcaaagtacattgacgaagcctgactgcgtacgacaatgtaccctatagtgagtcgtattaccggcc

SEQ ID NO: 89 - DNA, 144 bp, synthetic construct
  source 1..144: mol_type = other DNA
gccccatcatgtgcctttccttgacgaagcctgacggtctcggatatgcaagcaggggtttgccttctttgtccttgtcctcgctgattgacgaagcctgactgcgtacgacaatgtaccctatagtgagtcgtattaccggcc

SEQ ID NO: 90 - DNA, 144 bp, synthetic construct
  source 1..144: mol_type = other DNA
gccccatcatgtgcctttccttgacgaagcctgacggtctcggatatgcaaaggggagggcaggtggatggtgatcatctgcaacaattgacgaagcctgactgcgtacgacaatgtaccctatagtgagtcgtattaccggcc

SEQ ID NO: 91 - DNA, 144 bp, synthetic construct
  source 1..144: mol_type = other DNA
gccccatcatgtgcctttccttgacgaagcctgacggtctcggatatgcaaagccccgagaagactcgtccaaaggcgtagaaccgattgacgaagcctgactgcgtacgacaatgtaccctatagtgagtcgtattaccggcc

SEQ ID NO: 92 - DNA, 144 bp, synthetic construct
  source 1..144: mol_type = other DNA
gccccatcatgtgcctttccttgacgaagcctgacggtctcggatatgcaatcaggtagaggtcctccttcttcccaggggtatagattgacgaagcctgactgcgtacgacaatgtaccctatagtgagtcgtattaccggcc

SEQ ID NO: 93 - DNA, 144 bp, synthetic construct
  source 1..144: mol_type = other DNA
gccccatcatgtgcctttccttgacgaagcctgacggtctcggatatgcaatgggctccacgtagcggcccatcatcaagattgttattgacgaagcctgactgcgtacgacaatgtaccctatagtgagtcgtattaccggcc

SEQ ID NO: 94 - DNA, 144 bp, synthetic construct
  source 1..144: mol_type = other DNA
gccccatcatgtgcctttccttgacgaagcctgacggtctcggatatgcaaacgaggcccacaatgttcccacaaggcacatcctcattgacgaagcctgactgcgtacgacaatgtaccctatagtgagtcgtattaccggcc

SEQ ID NO: 95 - DNA, 144 bp, synthetic construct
  source 1..144: mol_type = other DNA
gccccatcatgtgcctttccttgacgaagcctgacggtctcggatatgcaatcatcacccgcatgttgtgcgcgtgctcgaaggtgattgacgaagcctgactgcgtacgacaatgtaccctatagtgagtcgtattaccggcc

SEQ ID NO: 96 - DNA, 144 bp, synthetic construct
  source 1..144: mol_type = other DNA
gccccatcatgtgcctttccttgacgaagcctgacggtctcggatatgcaatccacggccactctgacaacagggctgacgctgaaattgacgaagcctgactgcgtacgacaatgtaccctatagtgagtcgtattaccggcc

SEQ ID NO: 97 - DNA, 144 bp, synthetic construct
  source 1..144: mol_type = other DNA
gccccatcatgtgcctttccttgacgaagcctgacggtctcggatatgcaagcgatgatatgctctcccgactcctcgatgatgcaattgacgaagcctgactgcgtacgacaatgtaccctatagtgagtcgtattaccggcc

SEQ ID NO: 98 - DNA, 144 bp, synthetic construct
  source 1..144: mol_type = other DNA
gccccatcatgtgcctttccttgacgaagcctgacggtctcggatatgcaaaccgtctcgcggtacgagacgaccgggtcagatttattgacgaagcctgactgcgtacgacaatgtaccctatagtgagtcgtattaccggcc

SEQ ID NO: 99 - DNA, 144 bp, synthetic construct
  source 1..144: mol_type = other DNA
gccccatcatgtgcctttccttgacgaagcctgacggtctcggatatgcaaggacttggagaggcagagcacgttcgactcttcacattgacgaagcctgactgcgtacgacaatgtaccctatagtgagtcgtattaccggcc

SEQ ID NO: 100 - DNA, 144 bp, synthetic construct
  source 1..144: mol_type = other DNA
gccccatcatgtgcctttccttgacgaagcctgacggtctcggatatgcaagccgcgccttcatgtacagccggttgtgcttgttgattgacgaagcctgactgcgtacgacaatgtaccctatagtgagtcgtattaccggcc

SEQ ID NO: 101 - DNA, 144 bp, synthetic construct
  source 1..144: mol_type = other DNA
gccccatcatgtgcctttccttgacgaagcctgacggtctcggatatgcaacggccaccacactgtccttgatctcgttgaggtacattgacgaagcctgactgcgtacgacaatgtaccctatagtgagtcgtattaccggcc

SEQ ID NO: 102 - DNA, 144 bp, synthetic construct
  source 1..144: mol_type = other DNA
gccccatcatgtgcctttccttgacgaagcctgacggtctcggatatgcaatgacgggcagataggccttgaccacaaacatggggattgacgaagcctgactgcgtacgacaatgtaccctatagtgagtcgtattaccggcc

SEQ ID NO: 103 - DNA, 144 bp, synthetic construct
  source 1..144: mol_type = other DNA
gccccatcatgtgcctttccttgacgaagcctgacggtctcggatatgcaaaatttgtccaggaagttgtccagggcagggatgccattgacgaagcctgactgcgtacgacaatgtaccctatagtgagtcgtattaccggcc

SEQ ID NO: 104 - DNA, 70 bp, synthetic construct
  source 1..70: mol_type = other DNA
atcctgaacatgccttgatcgatcgtaatcaaaggccgcagatttaatgagccattcgcagtttcactgt

SEQ ID NO: 105 - DNA, 69 bp, synthetic construct
  source 1..69: mol_type = other DNA
gaggagcgagcgaccaaaggaaccataactgacggttgtaacggtacccgttgagtggctgttcaggat

SEQ ID NO: 106 - DNA, 70 bp, synthetic construct
  source 1..70: mol_type = other DNA
agaggagcgagcgaccaaaggaaccataactgacggttgtaacggtacccgttgagtggctggcgagaat

SEQ ID NO: 107 - DNA, 25 bp, synthetic construct
  source 1..25: mol_type = other DNA
tgttcaggattttttattctcgcca

SEQ ID NO: 108 - DNA, 17 bp, synthetic construct
  modified_base 1: mod_base = OTHER; note = 5' Hexynyl
  modified_base 14: mod_base = OTHER; note = iAmMC6T (Amino-Modifier C6 dT)
  modified_base 16: mod_base = OTHER; note = iAmMC6T (Amino-Modifier C6 dT)
  modified_base 17: mod_base = OTHER; note = 3' Amino Modifier (3AmMO)
  source 1..17: mol_type = other DNA
gtcaggcttcgtctata

SEQ ID NO: 109 - DNA, 17 bp, synthetic construct
  modified_base 1: mod_base = OTHER; note = 5' Hexynyl
  modified_base 14: mod_base = OTHER; note = iAmMC6T (Amino-Modifier C6 dT)
  modified_base 16: mod_base = OTHER; note = iAmMC6T (Amino-Modifier C6 dT)
  modified_base 17: mod_base = OTHER; note = 3' Amino Modifier (3AmMO)
  source 1..17: mol_type = other DNA
gccactcaacgggttta

(19) United States
(12) Patent Application Publication
(10) Pub. No.: US 2026/0357326 A1
(43) Pub. Date: Nov. 12, 2026

(54) READ-AND-ANCHOR SPLIT PROBE STRATEGY FOR HIGH-SPECIFICITY MOLECULAR PROFILING

(71) Applicant: CALIFORNIA INSTITUTE OF TECHNOLOGY, Pasadena, CA (US)

(72) Inventors: Long CAI, Pasadena, CA (US); Chuqi LU, Pasadena, CA (US); Haiyun LIU, Pasadena, CA (US); Wenbin NIE, Pasadena, CA (US)

(21) Appl. No.: 19/439,271

(22) Filed: Jan. 2, 2026

Related U.S. Application Data
(60) Provisional application No. 63/741,406, filed on Jan. 2, 2025.
(60) Provisional application No. 63/741,401, filed on Jan. 2, 2025.
(60) Provisional application No. 63/822,120, filed on Jun. 11, 2025.

(51) Int. Cl. C12Q 1/6818; C12Q 1/6841

(52) U.S. Cl. CPC C12Q 1/6818; C12Q 1/6841

(57) ABSTRACT

This methods described in this application provide a split probe strategy to profile molecules in a cell. The methods described in this application also relate to the use of FRET and sequential Fluorescence In Situ Hybridization to map the proximity of molecular interactions.

### PRIORITY

This application claims priority to U.S. Provisional Application No. 63/741,401, filed on Jan. 2, 2025, and entitled “SEQFISH FRET HYBRIDIZATION,” U.S. Provisional Application No. 63/741,406, filed on Jan. 2, 2025, and entitled “READ-AND-ANCHOR SPLIT PROBE STRATEGY FOR HIGH-SPECIFICITY MOLECULAR PROFILING,” and U.S. Provisional Application No. 63/822,120, filed on Jun. 11, 2025, and entitled “READ-AND-ANCHOR SPLIT PROBE STRATEGY FOR HIGH-SPECIFICITY MOLECULAR PROFILING.” Each of the foregoing applications is incorporated herein by reference in its entirety.

### STATEMENT REGARDING FEDERALLY SPONSORED RESEARCH

This invention was made with government support under NIH Grant No. DP1NS131408 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 1, 2026, is named “439915.00149.xml” and is 114,959 bytes in size. The sequence listing contained in this .XML file is part of the specification and is hereby incorporated by reference herein in its entirety.

### FIELD OF INVENTION

The methods relate to a split probe strategy to profile molecules in a cell.

### BACKGROUND

Spatial transcriptomics has emerged as a powerful tool for high-throughput analysis of gene expression within the spatial context of tissues and cells. It has significant applications in areas such as biomarker analysis for diseases and the identification and classification of cells within tissues.

Pioneering methods, such as seqFISH+, enable the unbiased identification of cell types and their spatial organization within tissues, while also revealing subcellular mRNA localization patterns. Despite achieving a tenfold or greater improvement over existing methods in the number of mRNAs profiled and RNA barcodes detected per cell, this method is not amplified.

Amplification of the hybridization signal reduces imaging time and brings the signal above the tissue's autofluorescence background. In addition, amplification can further reduce the number of probes used to detect the targets, which is useful for nucleic acids and protein targets. A smaller number or a single affinity reagent can be useful in detecting short genes or non-coding functional elements, where designing a sufficient number of probes, such as the standard 24, becomes unfeasible. For example, this limitation is particularly pronounced for genomic regions like enhancers. Furthermore, it complicates sample preservation, as extended storage durations can lead to degradation and loss of critical DNA or RNA fragments, thereby compromising the utility and reliability of the samples for subsequent analysis. Lastly, single antibody labeling events are difficult to detect. Overcoming these challenges necessitates amplification methods to improve the sensitivity and efficiency of spatial methods.

However, amplification can result in a nonspecific signal. For example, when repeated cycles of secondary and tertiary amplifier hybridization were employed to achieve exponential signal amplification, with newly hybridized amplifiers covalently anchored to the sample surface, the amplifiers can bind to cells non-specifically. These non-specific amplifiers, which can be indistinguishable from true hybridization signals, are similarly anchored to the cell surface via click reactions and contribute to subsequent amplification cycles, thus potentially increasing noise signal intensity. Similar problems exist in other branched-DNA and rolling circle amplification (RCA) based approaches. The amount of amplification achievable is limited by how much nonspecific signal competes with a specific signal. In addition, the nonspecific binding of primary probes or amplifiers can become particularly problematic when reducing the number of probes per gene, highlighting the need for more refined approaches to mitigate non-specific signal amplification.

Building on this foundation, the technology is designed to achieve highly specific and efficient imaging while minimizing the number of probes required, thereby expanding its applicability to a broader range of complex and constrained biological systems.

### SUMMARY

This disclosure provides a split probe strategy to profile molecules in a cell.

In some embodiments, the method comprises contacting a molecular target in a cell with a primary reading fragment. In some embodiments, the method comprises linking the primary reading fragment to the molecular target to form a primary complex. In some embodiments, the method comprises contacting the primary complex with one or more secondary reading fragments. In some embodiments, the method comprises optionally, linking the secondary reading fragments to the primary reading fragment. In some embodiments, the method comprises optionally, contacting the secondary reading fragments with one or more tertiary reading fragments. In some embodiments, the method comprises optionally, linking the tertiary reading fragments to the secondary reading fragments. In some embodiments, the method comprises contacting the secondary and/or tertiary reading fragments, with one or more detectably labelled readout probes. In some embodiments, the method comprises washing the cell to remove non-covalently bound fragments or probes at any of the previous steps. In some embodiments, the method comprises, optionally, wherein any of previous steps are repeated. In some embodiments, the method comprises imaging the one or more readout probes to identify the position of the molecular target in the cell.

In some embodiments, the method comprises contacting a molecular target in a cell with a primary reading fragment. In some embodiments, the method comprises contacting the primary reading fragment with one or more secondary reading fragments. In some embodiments, the method comprises optionally, contacting the secondary reading fragments with one or more tertiary reading fragments. In some embodiments, the method comprises contacting the secondary or tertiary reading fragments, with one or more detectably labelled readout probes, the detectably labelled readout probes capable of interacting with the secondary or tertiary reading fragments. In some embodiments, the method comprises washing the cell to remove non-covalently bound fragments or probes at any of the previous steps. In some embodiments, the method comprises optionally, repeating any of the previous steps. In some embodiments, the method comprises imaging the one or more readout probes to identify the position of the molecular target in the cell.

In some embodiments, the method comprises contacting a cell with an anchor probe, wherein the anchor probe interacts with a cellular component. In some embodiments, the method comprises contacting a molecular target with a primary reading fragment, wherein the primary reading fragment interacts with the molecular target; and a primary anchoring fragment, wherein the primary anchoring fragment interacts with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises washing the cell to remove non-covalently bound fragments at any of the previous steps. In some embodiments, the method comprises optionally, repeating any of the previous steps. In some embodiments, the method comprises contacting the primary probes with one or more detectably labelled readout probes. In some embodiments, the method comprises imaging the one or more readout probes to identify the position of the molecular target in the cell.

In some embodiments, the method comprises contacting a cell with an anchor probe, wherein the anchor probe interacts with a cellular component. In some embodiments, the method comprises contacting a molecular target with a primary reading fragment, wherein the primary reading fragment interacts with the molecular target; and a primary anchoring fragment, wherein the primary anchoring fragment interacts with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and one or more tertiary anchoring fragments. In some embodiments, the method comprises linking the tertiary reading fragments and tertiary anchoring fragments to form a tertiary probe. In some embodiments, the method comprises washing the cell to remove non-covalently bound fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a cell with an anchor probe, wherein the anchor probe hybridizes with a cellular component. In some embodiments, the method comprises contacting a molecular target with a primary reading fragment, wherein the primary reading fragment hybridizes with the molecular target; and a primary anchoring fragment, wherein the primary anchoring fragment hybridizes with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and one or more tertiary anchoring fragments. In some embodiments, the method comprises linking the tertiary reading fragments and tertiary anchoring fragments to form a tertiary probe. In some embodiments, the method comprises washing the cell to remove non-covalently bound fragments at any of the previous steps. In some embodiments, the method comprises optionally, repeating any of the previous steps are repeated. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment interacts with the molecular target; and an primary anchoring fragment, wherein the primary anchoring fragment interacts with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and tertiary anchoring fragments. In some embodiments, the method comprises linking the tertiary reading fragments and tertiary anchoring fragments to form tertiary probes. In some embodiments, the method comprises washing the cell to remove non-covalently bound fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment interacts with the molecular target; and an primary anchoring fragment, wherein the primary anchoring fragment interacts with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises washing the cell to remove non-covalently bound fragments at any of the previous steps; In some embodiments, the method comprises optionally, repeating any of the previous steps. In some embodiments, the method comprises contacting the primary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment hybridizes with the molecular target; and an primary anchoring fragment, wherein the primary anchoring fragment hybridizes with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises washing the cell to remove non-covalently bound fragments at any of the previous steps. In some embodiments, the method comprises contacting the primary probe with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment hybridizes with the molecular target; and an primary anchoring fragment, wherein the primary anchoring fragment hybridizes with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and tertiary anchoring fragments. In some embodiments, the method comprises linking the tertiary reading fragments and tertiary anchoring fragments to form tertiary probes. In some embodiments, the method comprises washing the cell to remove non-covalently bound fragments at any of the previous steps. In some embodiments, the method comprises optionally, repeating any of previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a protein in a cell with one or more reading antibodies and one or more anchoring antibodies, wherein the reading antibody is conjugated to one or more oligonucleotides. In some embodiments, the method comprises linking the reading antibodies and the anchoring antibodies. In some embodiments, the method comprises linking each anchoring antibody with an anchor probe, the anchor probe linked to a cellular component. In some embodiments, the method comprises optionally, amplifying the linked antibody and anchor with secondary probes, tertiary probes, and/or quaternary probes. In some embodiments, the method comprises washing the cell to remove non-covalently bound antibodies. In some embodiments, the method comprises contacting the reading antibodies with detectably labelled readout probes, wherein the readout probes interact with the oligonucleotides conjugated to the reading antibodies. In some embodiments, the method comprises imaging the readout probe to identify the position of the protein in the cell.

In some embodiments, the method comprises contacting a molecular target in a cell with one or more reading antibodies and one or more anchoring probes. In some embodiments, the method comprises optionally, linking the reading antibodies and the anchoring probes. In some embodiments, the method comprises linking each anchoring probe with an anchor probe, the anchor probe linked to a cellular component. In some embodiments, the method comprises optionally, amplifying the linked antibody and anchor with secondary probes, tertiary probe, and/or quaternary probes. In some embodiments, the method comprises washing the cell to remove non-covalently bound antibodies and probes. In some embodiments, the method comprises contacting the reading antibodies with detectably labelled readout probes, wherein the readout probes bind to the reading antibodies. In some embodiments, the method comprises imaging the readout probes to identify the position of the protein in the cell.

In some embodiments, the method comprises contacting one or more primary probes hybridized to one or more molecular targets in a cell with one or more secondary reading fragments and one or more secondary anchoring fragments. In certain embodiments, each primary probe comprises an anchor probe, a primary anchoring fragment linked to the anchor probe, and a primary reading fragment linked to the primary anchoring fragment. In certain embodiments, each secondary reading fragment hybridizes with one of the primary probes and/or one of the secondary anchoring fragments, and wherein each secondary anchoring fragment hybridizes with one of the primary probes. In certain embodiments, the method comprises linking secondary reading fragments and secondary anchoring fragments to form secondary probes, the secondary probes hybridized to the primary probes. In certain embodiments, the method comprises contacting the secondary probes with one or more tertiary reading fragments and one or more tertiary anchoring fragments, wherein each tertiary reading fragment hybridizes with one of the secondary probes and/or one of the tertiary anchoring fragments, and wherein each tertiary anchoring fragment hybridizes with one of the secondary probes. In certain embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more oligonucleotides hybridized to one or more molecular targets in a cell with one or more secondary reading fragments and one or more secondary anchoring fragments, In certain embodiments, each secondary reading fragment hybridizes with one of the oligonucleotides and/or one of the secondary anchoring fragments. In certain embodiments, each secondary anchoring fragment hybridizes with one of the oligonucleotides. In some embodiments, the method comprises linking secondary reading fragments and secondary anchoring fragments to form secondary probes, the secondary probes hybridized to the oligonucleotides. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more primary probes hybridized to one or more molecular targets in a cell with one or more secondary reading fragments and one or more secondary anchoring fragments, In certain embodiments, each secondary reading fragment hybridizes with one of the primary probes and/or one of the secondary anchoring fragments. In certain embodiments, each secondary anchoring fragment hybridizes with one of the primary probes. In some embodiments, the method comprises linking secondary reading fragments and secondary anchoring fragments to form secondary probes, the secondary probes hybridized to the primary probes. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more antibody oligonucleotide conjugates interacting with one or more molecular targets in a cell with one or more secondary reading fragments and one or more secondary anchoring fragments, In certain embodiments, each secondary reading fragment hybridizes with one of the oligonucleotides and/or one of the secondary anchoring fragments. In certain embodiments, each secondary anchoring fragment hybridizes with one of the oligonucleotides. In some embodiments, the method comprises linking secondary reading fragments and secondary anchoring fragments to form secondary probes, the secondary probes hybridized to the oligonucleotides. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises detecting one or more primary probes interacting with a substrate, comprising steps of contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes. In some embodiments, the method comprises linking secondary reading fragments and secondary anchoring fragments to form secondary probes. In some embodiments, the method comprises optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes. In some embodiments, the method comprises optionally, linking tertiary reading fragments and tertiary anchoring fragments to form tertiary probes. In some embodiments, the method comprises optionally, linking secondary probes to primary probes at any of previous steps. In some embodiments, the method comprises optionally, linking tertiary probes to secondary probes and/or to the primary probes. In some embodiments, the method comprises optionally, washing the substrate to remove fragments and/or probes at any of the previous steps. In some embodiments, the method comprises optionally, repeating any of the previous steps. In some embodiments, the method comprises contacting primary, secondary, and/or tertiary probes with one or more readout probes. In some embodiments, the method comprises detecting the one or more readout probes to detect the one or more primary probes interacting with the substrate.

In some embodiments, the method comprises detecting one or more primary probes interacting with a substrate, comprising steps of contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes. In some embodiments, the method comprises linking secondary reading fragments and secondary anchoring fragments to form secondary probes. In some embodiments, the method comprises optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes. In some embodiments, the method comprises optionally, linking tertiary reading fragments and tertiary anchoring fragments to form tertiary probes. In some embodiments, the method comprises optionally, linking secondary probes to primary probes at any of previous steps. In some embodiments, the method comprises optionally, linking tertiary probes to secondary probes and/or to the primary probes. In some embodiments, the method comprises optionally, washing the substrate to remove fragments and/or probes at any of the previous steps. In some embodiments, the method comprises optionally, repeating any of the previous steps. In some embodiments, the method comprises, optionally, contacting primary, secondary, and/or tertiary probes with one or more readout probes. In some embodiments, the method comprises detecting the one or more primary probes interacting with the substrate

In some embodiments, the method comprises detecting one or more primary probes interacting with a substrate, comprising steps of contacting the one or more primary probes with secondary reading fragments and optionally, secondary anchoring fragments, wherein each secondary reading fragment interacts with one of the primary probes and/or one of the secondary anchoring fragments, and wherein each secondary anchoring fragment interacts with one or more secondary reading fragments. In some embodiments, the method comprises optionally, linking the secondary reading fragments and secondary anchoring fragments to form secondary bridge complexes. In some embodiments, the method comprises optionally, contacting the secondary reading fragments with tertiary reading fragments and optionally, tertiary anchoring fragments, wherein each tertiary reading fragment interacts with one of the secondary reading fragments and/or one of the tertiary anchoring fragments, and wherein each tertiary anchoring fragment interacts with one or more tertiary reading fragments. In some embodiments, the method comprises optionally, linking tertiary reading fragments and tertiary anchoring fragments to form tertiary bridge complexes. In some embodiments, the method comprises optionally, linking secondary reading fragments to primary probes at any of the previous steps. In some embodiments, the method comprises optionally, linking tertiary reading fragments to secondary reading fragments at any of the previous. In some embodiments, the method comprises optionally, washing the substrate to remove fragments and/or probes at any of the previous steps. In some embodiments, the method comprises optionally, repeating any of the previous steps. In some embodiments, the method comprises contacting primary probes, secondary reading fragments, secondary anchoring fragments, tertiary reading fragments, and/or tertiary anchoring fragments with one or more readout probes. In some embodiments, the method comprises detecting the one or more readout probes to detect the one or more primary probes interacting with the substrate.

In some embodiments, the method comprises detecting one or more primary probes interacting with a substrate, comprising steps of contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes. In some embodiments, the method comprises linking secondary reading fragments and secondary anchoring fragments to form secondary probes. In some embodiments, the method comprises optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes. In some embodiments, the method comprises optionally, linking tertiary reading fragments and tertiary anchoring fragments to form tertiary probes. In some embodiments, the method comprises optionally, linking secondary probes to primary probes at any of previous steps. In some embodiments, the method comprises optionally, linking tertiary probes to secondary probes and/or to the primary probes. In some embodiments, the method comprises optionally, washing the substrate to remove fragments and/or probes at any of the previous steps. In some embodiments, the method comprises optionally, repeating any of the previous steps. In some embodiments, the method comprises, optionally, contacting primary, secondary, and/or tertiary probes with one or more readout probes. In some embodiments, the method comprises detecting the one or more primary probes interacting with the substrate.

In some embodiments, a method is described comprising steps of contacting a cell comprising molecular targets with a plurality of primary probes, each primary probe interacting with a molecular target, so that the plurality comprises a first primary probe that interacts with a first molecular target in the cell, and a second primary probe that interacts with a second molecular target in the cell. In some embodiments, the method comprises forming, by contacting secondary probes to the plurality of primary probes, a first probe complex on the first primary probe and a second probe complex on the second primary probe. In some embodiments, the method comprises contacting a plurality of detectably labelled readout probes to binding sites on the first and second probe complexes. In some embodiments the plurality of detectably labelled readout probes comprises a first readout probe that interacts with the first probe complex, wherein the first readout probe comprises a fluorescence donor, and a second readout probe that interacts with the second probe complex, wherein the second readout probe comprises a fluorescence acceptor. In some embodiments, the method comprises imaging the cell so that the proximity between the first molecular target and the second molecular target is detected from the interaction of the fluorescent donor and fluorescent acceptor.

In some embodiments, the method comprises contacting a cell comprising molecular targets with a plurality of primary probes, each primary probe interacting with a molecular target. In certain embodiments, the plurality of primary probes comprises a first primary probe that interacts with a first molecular target in the cell and a second primary probe that interacts with a second molecular target in the cell. In some embodiments, the method comprises contacting the primary probes with a plurality of secondary probes, wherein the plurality of secondary probes comprises a first secondary probe that interacts with the first primary probe and a second secondary probe that interacts with the second primary probe. In some embodiments, the method comprises optionally, contacting the secondary probes with a plurality of tertiary probes, wherein the plurality of tertiary probes comprises a first tertiary probe that interacts with the first secondary probe, and a second tertiary probe that interacts with the second secondary probe. In some embodiments, the method comprises optionally, contacting the tertiary probes with a plurality of quaternary probes after, wherein the plurality of quaternary probes comprises a first quaternary probe that interacts with the first tertiary probe or the first secondary probe; and a second quaternary probe that interacts with the second tertiary probe or the second secondary probe. In some embodiments, the method comprises optionally, repeating any of the previous contacting steps, wherein the secondary, tertiary, or quaternary probes comprise readout probe binding sites. In some embodiments, the method comprises contacting the secondary, tertiary, or quaternary probes with a plurality of readout probes capable of detection. In some embodiments, the plurality of readout probes comprises a first readout probe that interacts with the first secondary probes, tertiary probes, or quaternary probes. In some embodiments, the first readout probe comprises a moiety that is a fluorescent donor and a second readout probe that interacts with the second secondary probes, tertiary probes, or quaternary probes. In some embodiments, the second readout probe comprises a moiety that is a fluorescent acceptor. In some embodiments, the method comprises imaging the cell so that the proximity between the first molecular target and the second molecular target is detected from the interaction of the fluorescent donor and fluorescent acceptor.

The above methods have potential applications in the following scenarios:

Profiling the Spatial Distribution of T Cells with Distinct Human Antigen Receptors. This method leverages the detailed analysis of VDJ combinations to map specific target immune cell distributions across various tissues. Decoding the unique VDJ rearrangements in T cells, enables the precise identification and spatial profiling of auto-reactive T cells in autoimmune diseases such as psoriasis, vitiligo, and lupus. Additionally, this approach facilitates the characterization of tumor-associated immune responses by tracking immune cell infiltration and activation within tumor microenvironments, offering a tool for understanding immune dynamics in both pathological and therapeutic contexts.

Tracking Splicing Variants. This method offers the potential to map splice variants in cells by tracking intron retention and splicing events, providing insights into the molecular decision-making processes that govern gene expression. By enabling high-resolution profiling of intron splicing variants, particularly in complex systems like the brain, the approach could uncover regulatory mechanisms underlying neural development, function, and diseases. This capability is useful for studying alternative splicing events associated with neurological disorders. The method offers a tool for understanding gene regulation at the splicing level.

Deciphering Transcriptional States by Mapping Enhancer RNA. Enhancers are key regulatory elements driving tissue-specific gene expression, with over 400,000 putative enhancers forming intricate networks of enhancer-promoter interactions. The complexity and vast number of enhancers present significant challenges for traditional probe-based analysis, particularly in the context of diseases involving chromosomal rearrangements, genetic variations, and epigenetic modifications. This method has the potential to address these limitations by enabling high-resolution mapping of enhancer-promoter interactions, transcription factor-enhancer dynamics, and intron-RNA interactions, without requiring extensive probe libraries. The method could simplify the investigation of regulatory networks and provide valuable insights into enhancer-mediated gene regulation in cancer and developmental disorders.

The method offers the potential to target various types of RNAs. For instance, the method offers the potential to target small RNAs, microRNAs, and lncRNAs,

The method offers the potential to multiplex a large number of molecules. The method offers the potential to multiplex 10, 100, 1000, 10,000; 100,000; 1,000,000; 10,000,000; or 100,000,000 million types of endogenous and exogenously introduced molecules. These molecules may include nucleic acid barcodes.

The method offers advantages to reduce oligonucleotide aggregates. The use of oligonucleotides with crosslinker groups can be “sticky” in cells and form aggregates. This can lead to nonspecific binding and false positive amplifications in the cell when their concentration is high. A high total amplifier with crosslinker concentrations beyond 1 μM can lead to amplifier aggregate formation in samples. These aggregates are less likely to be observed with crosslinker-free amine control oligonucleotides.

One of the benefits of the secondary and tertiary anchoring fragments described herein is that only one or a few anchoring fragments are required for all the secondary or tertiary reading fragments. For example, if an experiment uses 60 amplifiers (reading fragments), and if the method uses anchoring fragments, then only 50 nM of a single anchoring fragment need to be used. In contrast, without anchoring fragments, one would need to have 60 reading fragments, each with a crosslinker group. This would yield a total concentration of 3 μM for all of the oligos with crosslinker groups. At that concentration, the reading fragments can cause stickiness and aggregation artifacts.

Crosslinking amplifiers with reading and anchoring fragments can maintain a compact structure and reduce nonspecific binding to improve the resolution of dense multiplexed signals. An amplified signal may arise from a single molecule of the target molecule. This is helpful for decoding a large number of molecular counts per cell. For example, if the amplified signal across different barcoding rounds for the same target is dispersed across 500 nanometers, then a (10 μm)³ cell would only be able to accommodate 8000 total barcodes per cell. Additional nonspecific amplified signals could further reduce this capacity by occupying optical space in the cell. This ceiling can limit the detection efficiency of many multiplexing experiments. However, if the amplified structures are compact (within 100 nm) and have a low number of non-specifically amplified signals, then 1,000,000 barcodes could fit into a cell. Having a lower nonspecific amplification from the reading and anchoring methods described herein allows a signal amplification of greater than 50-fold, and maintains the compact structure needed to decode a large number of amplified barcodes in single cells.

The methods disclosed herein demonstrated that FRET can be detected robustly in cells with two or more targets in close physical proximity when amplified. It was not clear apriori that FRET can occur across two aggregates of fluorophores (donors and acceptors) in assembled structures in cells. Homo-FRET and other energy transfer mechanisms could dissipate the FRET transfer across from donors to acceptors in such aggregates. However, significant FRET signals were observed, indicating efficient energy transfers from donors of one aggregate to the acceptors of the other aggregate.

The methods described herein can be used to analyze chromosome histone-DNA interactions. Combined with an advanced amplification method, the methods described herein can be used to study the interaction between histone markers/modifications and DNA loci inside the chromosome, as histone markers play an important role in gene expression regulation. Tumor tissue and wild-type tissue can be compared to determine if there are any differences in these interactions.

Combined with an advanced amplification method, the methods described herein can be used to study splicing factors and spliceosomes. Introns, splicing factors, and spliceosomes can be targeted to study their interactions inside tumor tissues and understand the mechanisms of splicing events, such as alternative splicing and intron retention. These methods also enable splice-profiling and allows us to observe how spliceosomes interact with RNAs.

The methods described herein can be used to study spatial chromatin interactions, such as those mediated by enhancers and promoters, or chromatin looping complexes like CTCF and cohesin as well as enhancer RNA.

Additionally, barcodes can be generated to encode the interactions within a complex, structured condensate to study these interactions in a multiplex fashion. These methods can be applied in a mouse brain to investigate condensates within the nuclear condensates. By designing multiple probes for the target, we can sequentially “walk” along it to probe the interactions one by one.

The methods disclosed herein can be used to study how proteins and RNA interact to form phase-separated compartments in cells such as stress granules and nucleoli.

The methods described herein can be used to study how circular RNA interacts with other proteins, RNAs.

Using barcoding scheme and amplification techniques, this method can be used to study thousands of interaction pairs including DNA-protein, RNA-protein, and protein-protein interactions in the same cells. As spatial information is preserved, these methods can be used to build a map that shows the connections between different types of molecular interactions.

### BRIEF DESCRIPTION OF THE DRAWINGS

FIG. 1: Overview of the “Read-and-Anchor Split Probe” method. Schematic representation of the method's mechanism. Illustration of how this method enhances specificity compared to traditional approaches in the “Read only,” which would be washed out without the anchor probes.

FIG. 2: Overview of split-probe and split-barcode methods. (A) “Read-and-anchor split” primary probe scheme. (B) “Read-and-tether split” amplifier probe scheme. (C) “Read-and-tether split” antibody barcode scheme.

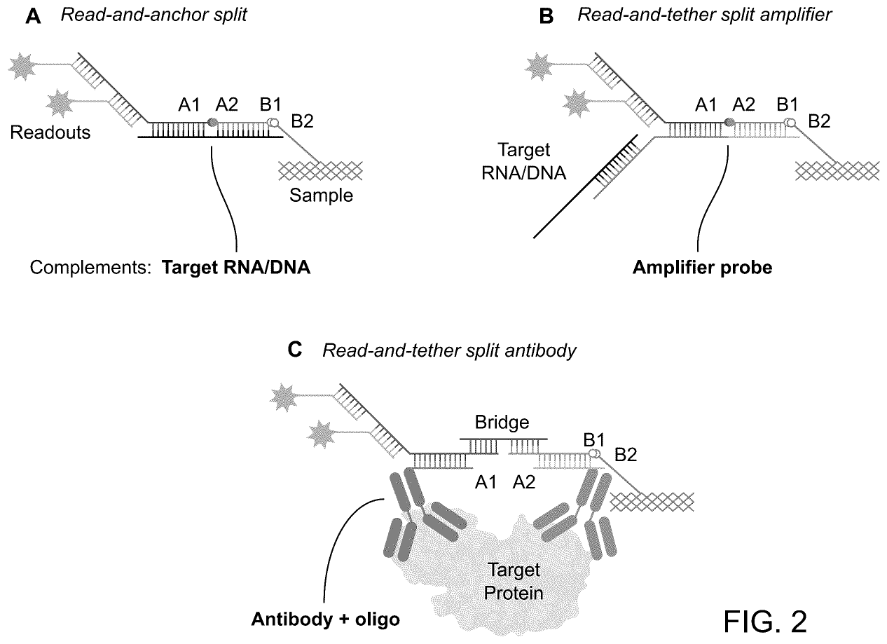

FIG. 3: Carboxyl-amine condensation proceeds with surprisingly high efficiency at the single molecule level in vivo. Images in the same channels are shown with the same contrast. Amine-modified readout oligos are hybridized to cells with primary probes and reacted with EDC to the carboxyl groups in the cell. Colocalization is observed between the 647 and 561 channels before and after harsh formamide wash. Most of the signal is retained even after a 60% formamide wash, indicating that most of the readout oligos are crosslinked to the cell through the EDC condensation reaction.

FIG. 4: Results of double crosslinking amplification in vivo.

FIG. 5: Shows a scheme by which the primary, secondary, tertiary, or quaternary fragments/probes interact with each other.

FIG. 6: Overview of probes stacking together. (A) the readout probe interacts with the tertiary probe, which in turn interacts with the secondary probe, which in turn interacts with the primary probe, which in turn interacts with the molecular target. (B) The readout probe partially interacts with the tertiary probe, which in turn partially interacts with the secondary probe, which in turn partially interacts with the primary probe, which in turn partially interacts with the molecular target. The dashed lines represent hydrogen bonding between the probes.

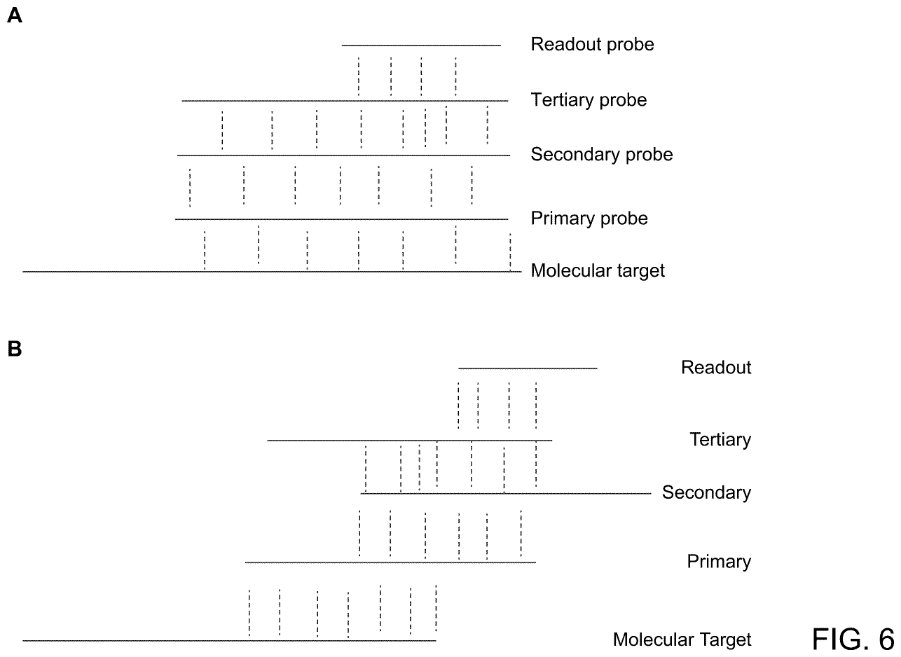

FIG. 7: Confocal microscopy images of Example 2 after amplification. From left to right, the three panels correspond to the 647 nm, 561 nm, and 488 nm channels. The upper row presents images of the entire cell, while the lower row shows a magnified view of a smaller region. As illustrated, the amplification procedure yields clear fluorescent signals with excellent colocalization across channels.

FIG. 8: Confocal microscopy images of Example 3 after amplification. From left to right, the panels correspond to the 647 nm and 561 nm fluorescence channels. The upper row shows images of the entire cell, whereas the lower row displays a magnified view of a selected subcellular region. The amplification produced bright fluorescent signals with strong colocalization between the two channels.

FIG. 9: Confocal microscopy images of Example 4 after amplification. From left to right, the panels correspond to the 647 nm and 561 nm fluorescence channels. The upper row shows images of the entire cell, whereas the lower row displays a magnified view of a selected subcellular region. The amplification produced bright fluorescent signals with strong colocalization between the two channels.

FIG. 10: Confocal microscopy images of Example 5 after amplification. From left to right, the panels correspond to the 647 nm, 561 nm, and 488 nm fluorescence channels. The first row shows the full field of view, the second row presents a magnified region containing several cells, and the third row displays a further enlarged view highlighting individual fluorescent puncta. The amplification procedure generated bright signals with strong colocalization across the three channels.

FIG. 11: Multiplex amplification Example of 9 highly expressed genes with anchor and read probes. The figures show the same cell with the 8 genes read out sequentially with 2 channels each. A zoom-in view of the boxed region in the first panel is displayed below each corresponding single-cell image. Colocalization rates exceed 80% for all eight genes.

FIG. 12: Amplified intron imaging of Rpl6 with 4 probes and amplification by anchor and read probes. Panel A shows that this method can detect active transcription sites of Rpl6, while Panel B shows the corresponding DAPI-stained image of the same field of view.

FIG. 13: (A) Schematic illustrates an overview of read-anchor scheme for interaction detection with the grab configuration. The top row shows that when Primary A and Primary B Grab probes are bound to the target of interest, a portion of them are complementary to each other, thus applying a physical constraint that brings them spatially close together. This dramatically increases the local concentration of azide and alkyne groups on the terminus of the read (or called amplifier) probe (green) and the terminus of the anchor probe (pink), enabling them to covalently link, or “click,” together under copper catalysis. Next, 365 nm UV light is used to activate the photo-crosslinker on the other terminus of the anchor probe, fixing the read-anchor probe complex to the cell sample. This ensures the complex stays in-situ after a harsh wash and allows readout probes to bind. The chemical click and photo-crosslinking steps working together serve as an “AND” gate; thus, a successful readout requires the completion of both reactions. Any incomplete complexes, as shown in the second and third rows, will be washed away or lack the necessary readout sites for probe binding. (B) Schematic illustrates an overview of read-anchor scheme for the loop configuration. The top row shows that when Primary A and Primary B Loop probes are bound to the target of interest alongside a Loop Bridge (shown in orange), the Loop Bridge binds to both primary probes, thus applying a physical constraint that brings them spatially close together. This dramatically increases the local concentration of azide and alkyne groups on the terminus of the amplifier probe (shown in green) and the terminus of the anchor probe (shown in pink), enabling them to covalently link, or “click,” together under copper catalysis. Next, 365 nm UV light is used to activate the photo-crosslinker on the other terminus of the anchor probe, fixing the amplifier-anchor probe complex to the cell sample. This ensures the complex stays in-situ after a harsh wash and allows readout probes to bind. The chemical click and photo-crosslinking steps working together serve as an “AND” gate; thus, a successful readout requires the completion of both reactions. The bottom row shows that in the absence of the Loop Bridge, the two primary probes remain free floating and too distant for the azide and alkyne groups to click effectively; consequently, the un-clicked amplifier probe is washed away, resulting in no readout signal.

FIG. 14: Fluorescence imaging demonstrates proximity dependent detection for the grab configuration after harsh wash, i.e. in 65% formamide in 37 °C for one hour. Microscopy images showing NIH3T3 cells hybridized under different probes. The top row shows cells hybridized with Primary A probe only. The bottom row shows cells hybridized with both Primary A and Primary B probes, which in this case are hybridized against the same target rRNA. The left column displays DAPI staining to visualize cell nuclei. The right column displays the fluorescent signal from the Amplifier readout. Amplifier signal is detected in the bottom row where both primary probes are present, but not in the top row where only Primary A is present. Scale bar denotes 10 μm.

FIG. 15: Fluorescence imaging demonstrating proximity dependent detection for the loop configuration after harsh wash, i.e. in 65% formamide in 37 °C for one hour. Microscopy images showing NIH3T3 cells hybridized under different probes. The top row shows cells hybridized with Primary A probe only. The middle row shows cells hybridized with Primary A and Primary B probes but without Loop Bridge. The bottom row shows cells hybridized with both Primary A, Primary B probes and Loop Bridge. The left column displays DAPI staining to visualize cell nuclei. The right column displays the fluorescent signal from the Amplifier readout. Amplifier signal is detected in the bottom row where both primary probes and loop bridge are present, but not in the top and middle row where there is no loop bridge. Scale bar denotes 10 μm.

FIG. 16: Bridge anchoring scheme. An anchoring sequence (bridge) can bridge across two secondary reading fragments such that when only both reading fragments are present the bridge retained. For example, the bridge may comprise crosslinkers that can be used to crosslink the anchoring fragment in the cell as well as crosslinking the anchoring fragment to the reading fragments. Iteration of this process to tertiary reading fragments and anchoring fragments allows amplification of the signal dependent on two or more adjacent reading fragment sequences (B and C). Proximity of two targets can be detected using this scheme with the anchoring fragment linking the probes interacting with each of the targets (D).

FIG. 17: (A) shows the donor readout probes are hybridized to amplifier, R3, and the acceptor readout probes are hybridized to amplifier, R1. FRET signals could be detected from the interaction between R1 and R3. (B) shows the macroscopic view of the two amplifiers on the split primary probs after 12 rounds of amplification, each amplified structure has roughly a diameter of 45 nm. This figure is not drawn to scale.

FIG. 18: shows the images of three different rounds of hybridization using four different channels and the contrast settings are the same for each individual channel. The zoomed in region emphasizes the intensity difference of the FRET channel when both the donor and acceptor are at presence on the amplifier.

FIG. 19: shows the schematic of how acceptor leakage and donor leakage into the FRET channel are calculated.

FIG. 20: shows a comprehensive analysis of the FRET experiment described in the text, showing the result of acceptor leak, donor leak, FRET intensity ratio and donor intensity drop when both donor and acceptor are at present.

FIG. 21: shows scheme by which the primary, secondary, tertiary, or quaternary probes interact with each other.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### DEFINITIONS

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 10000 nucleotides in length. In certain embodiments, the range can be any number in between 2 to 10000 nucleotides. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, and from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length. In some embodiments, the oligonucleotide is up to 40 nucleotides in length

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, etc.). For example, a probe can include a nucleic acid molecule, an oligonucleotide, a protein (e.g., an antibody or an antigen binding sequence), or combinations thereof. For example, a protein probe may be connected with one or more nucleic acid molecules to form a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal. In some embodiments, the probe refers to antibodies or small proteins.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces, etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to a nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As used herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As used herein, the term “one or more” generally refers to a number range in a set with at least one object in the set. In certain embodiments, the term “one or more” refers to 1, 2, 3, 4, 5, 6, 7, 8, 9, 10, 11, 12, 13, 14, 15, 16, 17. 18, 19, 20, 25, 30, 35, 40, 45, 50, 60, 70, 80, 90, 100, 150, 200, 250, 300, 350, 400, 450, 500, 600, 700, 800, 900, or 1,000,000,000 objects in a set. In certain embodiments, the term “one or more” refers to a range between 1 to 1000 objects in a set. In certain embodiments, the term “one or more” refers to a range between 1 to 10,000 objects in a set. In certain embodiments, the term “one or more” refers to a range between 1 to 100,000 objects in a set. In certain embodiments, the term “one or more” refers to a range between 1 to 1,000,000 objects in a set. In certain embodiments, the term “one or more” refers to a range between 1 to 10,000,000 objects in a set. In certain embodiments, the term “one or more” refers to a range between 1 to 100,000,000 objects in a set. In certain embodiments, the term “one or more” refers to a range between 1 to 500,000,000 objects in a set. For instance, using “one or more” oligonucleotides may refer to using 1 to 1000 oligonucleotides, or any number or range in between.

As used herein, the term “two or more” generally refers to a number range in a set with at least one object in the set. In certain embodiments, the term “two or more” refers to 2, 3, 4, 5, 6, 7, 8, 9, 10, 11, 12, 13, 14, 15, 16, 17. 18, 19, 20, 25, 30, 35, 40, 45, 50, 60, 70, 80, 90, 100, 150, 200, 250, 300, 350, 400, 450, 500, 600, 700, 800, 900, or 1,000,000,000 objects in a set. In certain embodiments, the term “two or more” refers to a range between 2 to 1000 objects in a set. In certain embodiments, the term “two or more” refers to a range between 2 to 100,000 objects in a set. In certain embodiments, the term “two or more” refers to a range between 2 to 1,000,000 objects in a set. In certain embodiments, the term “two or more” refers to a range between 2 to 10,000,000 objects in a set. In certain embodiments, the term “two or more” refers to a range between 2 to 100,000,000 objects in a set. In certain embodiments, the term “two or more” refers to a range between 2 to 500,000,000 objects in a set. For instance, using “two or more” oligonucleotides may refer to using 2 to 1000 oligonucleotides, or any number or range in between.

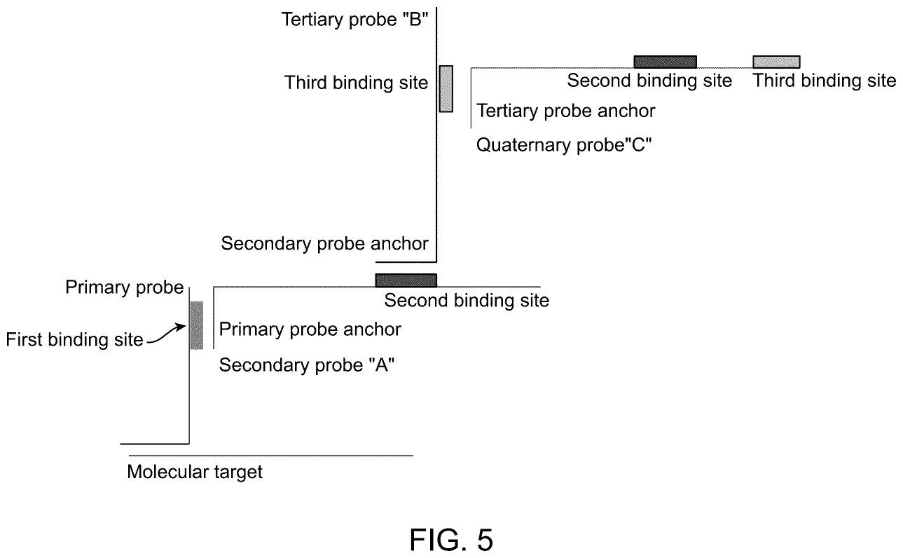

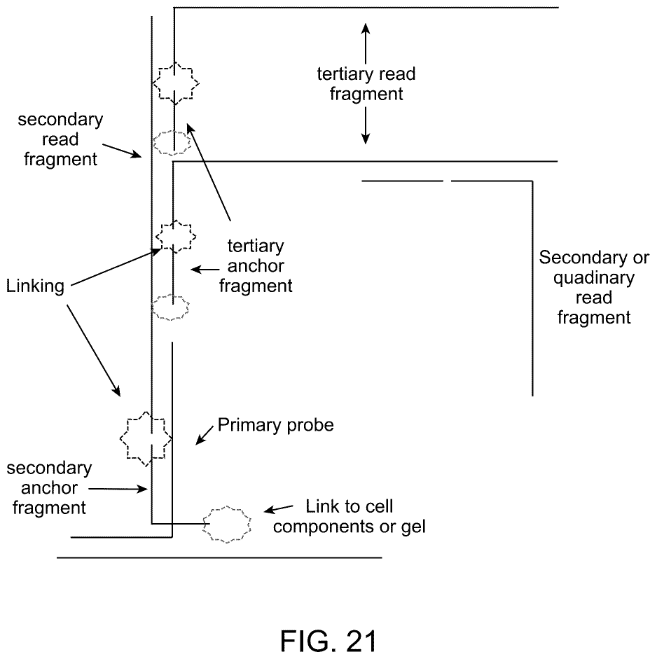

As used herein, the term “amplification” refers to the exponential growth of a molecule. As described with the specification examples and methods section and shown in FIG. 5, and/or FIG. 21, the use of secondary, tertiary, and quaternary probes can amplify the primary probe/probes.

As used herein, the terms “binding”, “interacting”, or “interaction” refer to the association of two or more molecules. In some embodiments, “binding” or “interactions” are the result of electrostatic forces, hydrogen bonding, der Waals forces, salt bridges, and the hydrophobic effect. In certain embodiments, “interacting” or “interaction” refers to the interaction between two or more molecules. In certain embodiments, “interacting” or “interaction” refers to the hybridization of two nucleic acids. In certain embodiments, the interaction may be indirect, wherein one molecule interacts with another molecule by way of one or more intermediate molecules. In certain embodiments, the interaction of two or more molecules refers to the cross-linking of the molecules.

As used herein, the term “with,” for instance, as used in the phrase “detecting one or more primary probes interacting ‘with’ a substrate” refers to “on” the surface of a substrate or “within” a substrate.

As used herein, the term “substrate” refers to a cell or a hydrogel.

As used herein, the term “linking” refers to joining two or more molecules together. In some embodiments, the linking forms one or more covalent bonds between two or more molecules. In some embodiments, the link may be formed by photo-crosslinking two or more molecules together. In some embodiments, the linking may join two or more molecules together by hydrogen bonding.

As used herein the term “photoreactive group” refers to a chemically inert compound or molecule that becomes reactive when exposed to ultraviolet or visible light, that allows the formation of a covalent bond between two molecules.

As used herein, the term “complementary sequence” refers to a region of one nucleotide sequence that can base pair with another nucleotide sequence. In some embodiments, the complementary sequence can base pair with 60%, 65%, 70%, 75%, 80%, 85%, 90%, 91%, 92%, 93%, 94%, 95%, 96%, 97%, 98%, 99%, or 100% of another nucleotide sequence. In certain embodiments, the complementary sequence is between 5-1000 nucleotides long.

As used herein, the term “linked” refers to two or more molecules having covalent bonds formed between them or an interaction between the two or more molecules.

As used herein, the term “functionalized cellular component” refers to a cellular component that has been modified to interact with the probes, anchors, anchoring fragments, and/or reading fragments described herein.

### Embodiments

In some embodiments, the method comprises contacting a molecular target in a cell with a primary reading fragment. In some embodiments, the method comprises linking the primary reading fragment to the molecular target to form a primary complex. In some embodiments, the method comprises contacting the primary complex with one or more secondary reading fragments. In some embodiments, the method comprises optionally, linking the secondary reading fragments to the primary reading fragment. In some embodiments, the method comprises optionally, contacting the secondary reading fragments with one or more tertiary reading fragments. In some embodiments, the method comprises optionally, linking the tertiary reading fragments to the secondary reading fragments. In some embodiments, the method comprises contacting the secondary and/or tertiary reading fragments, with one or more detectably labelled readout probes. In some embodiments, the method comprises washing the cell to remove non-covalently bound fragments or probes at any of the previous steps. In some embodiments, the method comprises, optionally, wherein any of previous steps are repeated. In some embodiments, the method comprises imaging the one or more readout probes to identify the position of the molecular target in the cell.

In some embodiments, the method comprises contacting a molecular target in a cell with a primary reading fragment. In some embodiments, the method comprises contacting the primary reading fragment with one or more secondary reading fragments. In some embodiments, the method comprises optionally, contacting the secondary reading fragments with one or more tertiary reading fragments. In some embodiments, the method comprises contacting the secondary or tertiary reading fragments, with one or more detectably labelled readout probes, the detectably labelled readout probes capable of interacting with the secondary or tertiary reading fragments. In some embodiments, the method comprises washing the cell to remove non-covalently bound fragments or probes at any of the previous steps. In some embodiments, the method comprises optionally, repeating any of the previous steps. In some embodiments, the method comprises imaging the one or more readout probes to identify the position of the molecular target in the cell.

In some embodiments, the method comprises contacting a cell with an anchor probe, wherein the anchor probe interacts with a cellular component. In some embodiments, the method comprises contacting a molecular target with a primary reading fragment, wherein the primary reading fragment interacts with the molecular target; and a primary anchoring fragment, wherein the primary anchoring fragment interacts with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises washing the cell to remove non-covalently bound fragments at any of the previous steps. In some embodiments, the method comprises optionally, repeating any of the previous steps. In some embodiments, the method comprises contacting the primary probes with one or more detectably labelled readout probes. In some embodiments, the method comprises imaging the one or more readout probes to identify the position of the molecular target in the cell.

In some embodiments, the method comprises contacting a cell with an anchor probe, wherein the anchor probe interacts with a cellular component. In some embodiments, the method comprises contacting a molecular target with a primary reading fragment, wherein the primary reading fragment interacts with the molecular target; and a primary anchoring fragment, wherein the primary anchoring fragment interacts with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and one or more tertiary anchoring fragments. In some embodiments, the method comprises linking the tertiary reading fragments and tertiary anchoring fragments to form a tertiary probe. In some embodiments, the method comprises washing the cell to remove non-covalently bound fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a cell with an anchor probe, wherein the anchor probe hybridizes with a cellular component. In some embodiments, the method comprises contacting a molecular target with a primary reading fragment, wherein the primary reading fragment hybridizes with the molecular target; and a primary anchoring fragment, wherein the primary anchoring fragment hybridizes with the molecular target, and wherein the primary anchoring fragment is capable of linking to the anchor probe. In some embodiments, the method comprises linking the primary reading fragment and the primary anchoring fragment to form a primary probe. In some embodiments, the method comprises optionally, linking the primary anchoring fragment and the anchor probe. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and one or more tertiary anchoring fragments. In some embodiments, the method comprises linking the tertiary reading fragments and tertiary anchoring fragments to form a tertiary probe. In some embodiments, the method comprises washing the cell to remove non-covalently bound fragments at any of the previous steps. In some embodiments, the method comprises optionally, repeating any of the previous steps are repeated. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment interacts with the molecular target; and an primary anchoring fragment, wherein the primary anchoring fragment interacts with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and tertiary anchoring fragments. In some embodiments, the method comprises linking the tertiary reading fragments and tertiary anchoring fragments to form tertiary probes. In some embodiments, the method comprises washing the cell to remove non-covalently bound fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment interacts with the molecular target; and an primary anchoring fragment, wherein the primary anchoring fragment interacts with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises washing the cell to remove non-covalently bound fragments at any of the previous steps; In some embodiments, the method comprises optionally, repeating any of the previous steps. In some embodiments, the method comprises contacting the primary probes with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment hybridizes with the molecular target; and an primary anchoring fragment, wherein the primary anchoring fragment hybridizes with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises washing the cell to remove non-covalently bound fragments at any of the previous steps. In some embodiments, the method comprises contacting the primary probe with one or more detectably labelled readout probes. In some embodiments, the method comprises imaging the one or more readout probes to identify the position of the molecular target in the cell.

In some embodiments, the method comprises contacting a molecular target in a cell with a primary reading fragment, wherein the primary reading fragment hybridizes with the molecular target; and an primary anchoring fragment, wherein the primary anchoring fragment hybridizes with the molecular target, and wherein the primary anchoring fragment is capable of linking to an anchor probe, optionally the anchor probe linked to a cellular component in the cell. In some embodiments, the method comprises linking the reading fragment and the anchoring fragment to form a primary probe. In some embodiments, the method comprises optionally, linking the anchoring fragment and the anchor probe to form a molecular target anchor. In some embodiments, the method comprises optionally, linking the primary probe to the cellular components. In some embodiments, the method comprises contacting the primary probe with one or more secondary reading fragments and one or more secondary anchoring fragments. In some embodiments, the method comprises linking the secondary reading fragments and secondary anchoring fragments to form secondary probes. In some embodiments, the method comprises contacting the secondary probes with one or more tertiary reading fragments and tertiary anchoring fragments. In some embodiments, the method comprises linking the tertiary reading fragments and tertiary anchoring fragments to form tertiary probes. In some embodiments, the method comprises washing the cell to remove non-covalently bound fragments at any of the previous steps. In some embodiments, the method comprises optionally, repeating any of previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting a molecular target in a cell with one or more reading antibodies and one or more anchoring probes. In some embodiments, the method comprises optionally, linking the reading antibodies and the anchoring probes. In some embodiments, the method comprises linking each anchoring probe with an anchor probe, the anchor probe linked to a cellular component. In some embodiments, the method comprises optionally, amplifying the linked antibody and anchor with secondary probes, tertiary probe, and/or quaternary probes. In some embodiments, the method comprises washing the cell to remove non-covalently bound antibodies and probes. In some embodiments, the method comprises contacting the reading antibodies with detectably labelled readout probes, wherein the readout probes bind to the reading antibodies. In some embodiments, the method comprises imaging the readout probes to identify the position of the protein in the cell.

In some embodiments, the method comprises contacting a protein in a cell with one or more reading antibodies and one or more anchoring antibodies, wherein the reading antibody is conjugated to one or more oligonucleotides. In some embodiments, the method comprises linking the reading antibodies and the anchoring antibodies. In some embodiments, the method comprises linking each anchoring antibody with an anchor probe, the anchor probe linked to a cellular component. In some embodiments, the method comprises optionally, amplifying the linked antibody and anchor with secondary probes, tertiary probes, and/or quaternary probes. In some embodiments, the method comprises washing the cell to remove non-covalently bound antibodies. In some embodiments, the method comprises contacting the reading antibodies with detectably labelled readout probes, wherein the readout probes interact with the oligonucleotides conjugated to the reading antibodies. In some embodiments, the method comprises imaging the readout probe to identify the position of the protein in the cell.

In some embodiments, the method comprises contacting one or more primary probes hybridized to one or more molecular targets in a cell with one or more secondary reading fragments and one or more secondary anchoring fragments. In certain embodiments, each primary probe comprises: an anchor probe, a primary anchoring fragment linked to the anchor probe, and a primary reading fragment linked to the primary anchoring fragment. In certain embodiments, each secondary reading fragment hybridizes with one of the primary probes and/or one of the secondary anchoring fragments, and wherein each secondary anchoring fragment hybridizes with one of the primary probes. In certain embodiments, the method comprises linking secondary reading fragments and secondary anchoring fragments to form secondary probes, the secondary probes hybridized to the primary probes. In certain embodiments, the method comprises contacting the secondary probes with one or more tertiary reading fragments and one or more tertiary anchoring fragments, wherein each tertiary reading fragment hybridizes with one of the secondary probes and/or one of the tertiary anchoring fragments, and wherein each tertiary anchoring fragment hybridizes with one of the secondary probes. In certain embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more oligonucleotides hybridized to one or more molecular targets in a cell with one or more secondary reading fragments and one or more secondary anchoring fragments, In certain embodiments, each secondary reading fragment hybridizes with one of the oligonucleotides and/or one of the secondary anchoring fragments. In certain embodiments, each secondary anchoring fragment hybridizes with one of the oligonucleotides. In some embodiments, the method comprises linking secondary reading fragments and secondary anchoring fragments to form secondary probes, the secondary probes hybridized to the oligonucleotides. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more primary probes hybridized to one or more molecular targets in a cell with one or more secondary reading fragments and one or more secondary anchoring fragments, In certain embodiments, each secondary reading fragment hybridizes with one of the primary probes and/or one of the secondary anchoring fragments. In certain embodiments, each secondary anchoring fragment hybridizes with one of the primary probes. In some embodiments, the method comprises linking secondary reading fragments and secondary anchoring fragments to form secondary probes, the secondary probes hybridized to the primary probes. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method comprises contacting one or more antibody oligonucleotide conjugates interacting with one or more molecular targets in a cell with one or more secondary reading fragments and one or more secondary anchoring fragments, In certain embodiments, each secondary reading fragment hybridizes with one of the oligonucleotides and/or one of the secondary anchoring fragments. In certain embodiments, each secondary anchoring fragment hybridizes with one of the oligonucleotides. In some embodiments, the method comprises linking secondary reading fragments and secondary anchoring fragments to form secondary probes, the secondary probes hybridized to the oligonucleotides. In some embodiments, the method comprises contacting the secondary probes with one or more tertiary reading fragments and one or more tertiary anchoring fragments. In certain embodiments, each tertiary reading fragment hybridizes with one of the secondary probes and/or one of the tertiary anchoring fragments. In certain embodiments, each tertiary anchoring fragment hybridizes with one of the secondary probes. In some embodiments, the method comprises linking tertiary reading fragments and tertiary anchoring fragments to form tertiary probes, the tertiary probes hybridized to the secondary probes. In some embodiments, the method comprises optionally, washing the cell to remove unlinked fragments at any of the previous steps. In some embodiments, the method comprises optionally, repeating any of the previous steps. In some embodiments, the method comprises contacting the secondary and/or tertiary probes with one or more detectably labelled readout probes. In some embodiments, the method comprises imaging the one or more readout probes to identify the position of the molecular target in the cell.

In some embodiments, the method of any of the previous embodiments comprises optionally, contacting the tertiary probes after any of the previous steps with quaternary reading fragments and quaternary anchoring fragments, wherein each quaternary reading fragment interacts with one of the tertiary probes and/or one of the quaternary anchoring fragments, and wherein each quaternary anchoring fragment interacts with one of the tertiary probes. In some embodiments, the method comprises optionally, linking quaternary reading fragments and quaternary anchoring fragments to form quaternary probes. In some embodiments, the method comprises contacting the primary, secondary, tertiary, and/or quaternary probes with one or more readout probes. In some embodiments, the method of any of the previous embodiments, are performed are performed concurrently with contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes.

In some embodiments, the method comprises detecting one or more primary probes interacting with a substrate, comprising steps of contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes. In some embodiments, the method comprises linking secondary reading fragments and secondary anchoring fragments to form secondary probes. In some embodiments, the method comprises optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes. In some embodiments, the method comprises optionally, linking tertiary reading fragments and tertiary anchoring fragments to form tertiary probes. In some embodiments, the method comprises optionally, linking secondary probes to primary probes at any of previous steps. In some embodiments, the method comprises optionally, linking tertiary probes to secondary probes and/or to the primary probes. In some embodiments, the method comprises optionally, washing the substrate to remove fragments and/or probes at any of the previous steps. In some embodiments, the method comprises optionally, repeating any of the previous steps. In some embodiments, the method comprises contacting primary, secondary, and/or tertiary probes with one or more readout probes. In some embodiments, the method comprises detecting the one or more readout probes to detect the one or more primary probes interacting with the substrate.

In some embodiments, the method comprises detecting one or more primary probes interacting with a substrate, comprising steps of contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes. In some embodiments, the method comprises linking secondary reading fragments and secondary anchoring fragments to form secondary probes. In some embodiments, the method comprises optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes. In some embodiments, the method comprises optionally, linking tertiary reading fragments and tertiary anchoring fragments to form tertiary probes. In some embodiments, the method comprises optionally, linking secondary probes to primary probes at any of previous steps. In some embodiments, the method comprises optionally, linking tertiary probes to secondary probes and/or to the primary probes. In some embodiments, the method comprises optionally, washing the substrate to remove fragments and/or probes at any of the previous steps. In some embodiments, the method comprises optionally, repeating any of the previous steps. In some embodiments, the method comprises, optionally, contacting primary, secondary, and/or tertiary probes with one or more readout probes. In some embodiments, the method comprises detecting the one or more primary probes interacting with the substrate.

In some embodiments, the method of any of the previous embodiments comprises optionally, contacting the tertiary probes after any of the previous steps with quaternary reading fragments and quaternary anchoring fragments, wherein each quaternary reading fragment interacts with one of the tertiary probes and/or one of the quaternary anchoring fragments, and wherein each quaternary anchoring fragment interacts with one of the tertiary probes. In some embodiments, the method comprises optionally, linking quaternary reading fragments and quaternary anchoring fragments to form quaternary probes. In some embodiments, the method comprises contacting the primary, secondary, tertiary, and/or quaternary probes with one or more readout probes. In some embodiments, the method of any of the previous embodiments, are performed are performed concurrently with contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes.

In some embodiments, the method comprises detecting one or more primary probes interacting with a substrate, comprising steps of contacting the one or more primary probes with secondary reading fragments and optionally, secondary anchoring fragments, wherein each secondary reading fragment interacts with one of the primary probes and/or one of the secondary anchoring fragments, and wherein each secondary anchoring fragment interacts with one or more secondary reading fragments. In some embodiments, the method comprises optionally, linking the secondary reading fragments and secondary anchoring fragments to form secondary bridge complexes. In some embodiments, the method comprises optionally, contacting the secondary reading fragments with tertiary reading fragments and optionally, tertiary anchoring fragments, wherein each tertiary reading fragment interacts with one of the secondary reading fragments and/or one of the tertiary anchoring fragments, and wherein each tertiary anchoring fragment interacts with one or more tertiary reading fragments. In some embodiments, the method comprises optionally, linking tertiary reading fragments and tertiary anchoring fragments to form tertiary bridge complexes. In some embodiments, the method comprises optionally, linking secondary reading fragments to primary probes at any of the previous steps. In some embodiments, the method comprises optionally, linking tertiary reading fragments to secondary reading fragments at any of the previous. In some embodiments, the method comprises optionally, washing the substrate to remove fragments and/or probes at any of the previous steps. In some embodiments, the method comprises optionally, repeating any of the previous steps. In some embodiments, the method comprises contacting primary probes, secondary reading fragments, secondary anchoring fragments, tertiary reading fragments, and/or tertiary anchoring fragments with one or more readout probes. In some embodiments, the method comprises detecting the one or more readout probes to detect the one or more primary probes interacting with the substrate.

In some embodiments, the method comprises detecting one or more primary probes interacting with a substrate, comprising steps of contacting the one or more primary probes with secondary reading fragments and optionally, secondary anchoring fragments, wherein each secondary reading fragment interacts with one of the primary probes and/or one of the secondary anchoring fragments, and wherein each secondary anchoring fragment interacts with one or more secondary reading fragments. In some embodiments, the method comprises optionally, linking the secondary reading fragments and secondary anchoring fragments to form secondary bridge complexes. In some embodiments, the method comprises optionally, contacting the secondary reading fragments with tertiary reading fragments and optionally, tertiary anchoring fragments, wherein each tertiary reading fragment interacts with one of the secondary reading fragments and/or one of the tertiary anchoring fragments, and wherein each tertiary anchoring fragment interacts with one or more tertiary reading fragments. In some embodiments, the method comprises optionally, linking tertiary reading fragments and tertiary anchoring fragments to form tertiary bridge complexes. In some embodiments, the method comprises optionally, linking secondary reading fragments to primary probes at any of the previous steps. In some embodiments, the method comprises optionally, linking tertiary reading fragments to secondary reading fragments at any of the previous. In some embodiments, the method comprises optionally, washing the substrate to remove fragments and/or probes at any of the previous steps. In some embodiments, the method comprises optionally, repeating any of the previous steps. In some embodiments, the method comprises, optionally, contacting primary probes, secondary reading fragments, secondary anchoring fragments, tertiary reading fragments, and/or tertiary anchoring fragments with one or more readout probes. In some embodiments, the method comprises detecting the one or more primary probes interacting with the substrate.

In some embodiments, the method of any of the previous embodiments comprises optionally, contacting the tertiary reading fragments during or after any of the previous steps with quaternary reading fragments and quaternary anchoring fragments, wherein each quaternary reading fragment interacts with one of the tertiary reading fragments and/or one of the quaternary anchoring fragments, and wherein each quaternary anchoring fragment interacts with one or more tertiary reading fragments. In some embodiments, the method comprises optionally, linking quaternary reading fragments and quaternary anchoring fragments to form quaternary bridge complexes. In some embodiments, the method comprises contacting the primary probes, secondary reading fragments, tertiary reading fragments, quaternary reading fragments, secondary anchoring fragments, tertiary anchoring fragments, and/or quaternary anchoring fragments with one or more readout probes.

In some embodiments, the method of any of the previous embodiments, are performed are performed concurrently with contacting the one or more primary probes with secondary reading fragments and optionally, secondary anchoring fragments, wherein each secondary reading fragment interacts with one of the primary probes and/or one of the secondary anchoring fragments, and wherein each secondary anchoring fragment interacts with one or more secondary reading fragments.

In some embodiments, a method is described comprising steps of contacting a cell comprising molecular targets with a plurality of primary probes, each primary probe interacting with a molecular target, so that the plurality comprises a first primary probe that interacts with a first molecular target in the cell, and a second primary probe that interacts with a second molecular target in the cell. In some embodiments, the method comprises forming, by contacting secondary probes to the plurality of primary probes, a first probe complex on the first primary probe and a second probe complex on the second primary probe. In some embodiments, the method comprises contacting a plurality of detectably labelled readout probes to binding sites on the first and second probe complexes. In some embodiments the plurality of detectably labelled readout probes comprise a first readout probe that interacts with the first probe complex, wherein the first readout probe comprises a fluorescence donor, and a second readout probe that interacts with the second probe complex, wherein the second readout probe comprises a fluorescence acceptor. In some embodiments, the method comprises imaging the cell so that the proximity between the first molecular target and the second molecular target is detected from the interaction of the fluorescent donor and fluorescent acceptor.

In some embodiments, the method comprises contacting a cell comprising molecular targets with a plurality of primary probes, each primary probe interacting with a molecular target. In certain embodiments, the plurality of primary probes comprises a first primary probe that interacts with a first molecular target in the cell and a second primary probe that interacts with a second molecular target in the cell. In some embodiments, the method comprises contacting the primary probes with a plurality of secondary probes, wherein the plurality of secondary probes comprises a first secondary probe that interacts with the first primary probe and a second secondary probe that interacts with the second primary probe. In some embodiments, the method comprises optionally, contacting the secondary probes with a plurality of tertiary probes, wherein the plurality of tertiary probes comprises a first tertiary probe that interacts with the first secondary probe, and a second tertiary probe that interacts with the second secondary probe. In some embodiments, the method comprises optionally, contacting the tertiary probes with a plurality of quaternary probes after, wherein the plurality of quaternary probes comprises a first quaternary probe that interacts with the first tertiary probe or the first secondary probe; and a second quaternary probe that interacts with the second tertiary probe or the second secondary probe. In some embodiments, the method comprises optionally, repeating any of the previous contacting steps, wherein the secondary, tertiary, or quaternary probes comprise readout probe binding sites. In some embodiments, the method comprises contacting the secondary, tertiary, or quaternary probes with a plurality of readout probes capable of detection. In some embodiments, the plurality of readout probes comprises a first readout probe that interacts with the first secondary probes, tertiary probes, or quaternary probes. In some embodiments, the first readout probe comprises a moiety that is a fluorescent donor and a second readout probe that interacts with the second secondary probes, tertiary probes, or quaternary probes. In some embodiments, the second readout probe comprises a moiety that is a fluorescent acceptor. In some embodiments, the method comprises imaging the cell so that the proximity between the first molecular target and the second molecular target is detected from the interaction of the fluorescent donor and fluorescent acceptor.

### Samples

In some embodiments, the method comprises analyzing samples, wherein the samples comprise bacterial cells, archaeal cells, eukaryotic cells, or a combination thereof. In some embodiments, the samples comprise tissues, cells, or extracts from cells. In some embodiments, the samples comprise cells obtained from patients. In some embodiments, the samples comprise fluids obtained from patients.

In some embodiments, the sample comprises molecular targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the molecular targets comprise subcellular features.

### Substrates, Molecular Targets, and Cellular Components

A wide variety of cellular components and molecular targets can be mapped by the methods disclosed herein.

In some embodiments, the method comprises detecting one or more primary probes interacting with a substate. In certain embodiments, the substrate is a cell. In certain embodiments, the substrate is a hydrogel.

In some embodiments, the substrate comprises one or more molecular targets. In some embodiments, the substrate comprises one to 1,000,000,000 molecular targets. In some embodiments, the substrate comprises 2 to 10,000 molecular targets. In some embodiments, the substrate comprises 5 to 10,000 molecular targets. In some embodiments, the substrate comprises 10 to 10,000 molecular targets. In some embodiments, the substrate comprises 20 to 10,000 molecular targets. In some embodiments, the substrate comprises 30 to 10,000 molecular targets. In some embodiments, the substrate comprises 50 to 10,000 molecular targets. In some embodiments, the substrate comprises 100 to 10,000 molecular targets. In some embodiments, the substrate comprises 200 to 10,000 molecular targets. In some embodiments, the substrate comprises 300 to 10,000 molecular targets. In some embodiments, the substrate comprises 400 to 10,000 molecular targets. In some embodiments, the substrate comprises 500 to 10,000 molecular targets. In some embodiments, the substrate comprises 700 to 10,000 molecular targets. In some embodiments, the substrate comprises 800 to 10,000 molecular targets.

In some embodiments, the substrate is linked to one or more molecular targets. In certain embodiments, the one or more molecular targets are the same. In certain embodiments, the one or more molecular targets are different.

In some embodiments, the substrate comprises one or more cellular components. In some embodiments, the substrate is linked to one or more cellular components. In certain embodiments, the one or more cellular components are the same. In certain embodiments, the one or more cellular components are different. In some embodiments, the substrate is a functionalized cellular component. In some embodiments, the substrate is embedded in the gel matrix. In some embodiments, the cellular components are selected from oligonucleotides, DNAs, RNAs, small molecules, glycans, peptides, proteins, antibodies, antibodies conjugated to oligonucleotides, organelles, subcellular compartments, and combinations thereof.

In some embodiments, the molecular targets are selected from proteins, modified proteins, transcripts, RNAs, DNAs, DNA loci, exogenous proteins, exogenous nucleic acids, hormones, carbohydrates, glycans, small molecules, biologically active molecules, and combinations thereof. In some embodiments, the molecular targets comprise subcellular features.

In certain embodiments, the DNAs of any of the previous embodiments comprise endogenous cellular DNAs. In certain embodiments, the DNAs of any of the previous embodiments comprise exogenous DNAs introduced into the cell. In certain embodiments, the exogenous DNAs of any of the previous embodiments introduced into the cell comprising of oligonucleotides.

In some embodiments, the cellular component is intracellularly distributed. In some embodiments, the cellular component is intercellularly distributed.

In some embodiments, the molecular targets are the same as the cellular components. In some embodiments, the molecular targets are different than the cellular components.

### Probes

In some embodiments, methods comprise using probes to locate one or more molecular targets. In some embodiments, the probes of any of the previous embodiments are selected from oligonucleotides, DNA, RNA, small molecules, peptides, proteins, antibodies, and combinations thereof.

In some embodiments, the method comprises primary probes, secondary probes, tertiary probes, and quaternary probes. In certain embodiments, the primary probes, secondary probes, and tertiary probes comprise anchoring fragments and reading fragments.

### Primary Probes

In some embodiments, the method comprises one or more primary probes interacting with the one or more molecular targets.

In some embodiments, the method comprises one or more primary probes interacting with one or more molecular targets by binding to one or more molecular targets. In some embodiments, the method comprises one or more primary probes interacting with the one or more molecular targets by hybridizing to one or more molecular targets.

In some embodiments, the method comprises primary probes that interact with different molecular targets. In some embodiments, the method comprises primary probes that interact with the same molecular targets. In some embodiments, the method comprises two or more primary probes that interact with the same molecular target. In some embodiments, the method comprises two or more primary probes that interact with different molecular targets. In some embodiments, the method comprises two or more primary probes are in close proximity to each other. In certain embodiments, the proximity of the two or more primary probes is between 0.1 to 100 nanometers.

In some embodiments, the one or more primary probes are selected from oligonucleotides, DNAs, RNAs, small molecules, glycans, peptides, proteins, antibodies, antibodies conjugated to oligonucleotides, and combinations thereof. In certain embodiments, the one or more primary probes are oligonucleotides. In certain embodiments, the one or more primary probes are antibodies conjugated to oligonucleotides.

In some embodiments, the primary probe is an oligonucleotide. In some embodiments, the primary probe is between 2 and 10000 nucleotides in length.

In some embodiments, the method comprises one to 10,000,000 primary probes interacting with one or more molecular targets. In some embodiments, the method comprises 2 to 100 primary probes interacting with one or more molecular targets. In some embodiments, the method comprises 5 to 100 primary probes interacting with one or more molecular targets. In some embodiments, the method comprises 10 to 100 primary probes interacting with one or more molecular targets. In some embodiments, the method comprises 15 to 100 primary probes interacting with one or more molecular targets. In some embodiments, the method comprises 20 to 100 primary probes interacting with one or more molecular targets. In some embodiments, the method comprises 30 to 100 primary probes interacting with one or more molecular targets. In some embodiments, the method comprises 50 to 100 primary probes interacting with one or more molecular targets. In some embodiments, the method comprises 75 to 100 primary probes interacting with one or more molecular targets. interacting with one or more molecular targets. In some embodiments, the method comprises 2 to 10,000,000 primary probes interacting with one or more molecular targets. In some embodiments, the method comprises 5 to 10,000,000 primary probes interacting with one or more molecular targets. In some embodiments, the method comprises 10 to 10,000,000 primary probes interacting with one or more molecular targets. In some embodiments, the method comprises 15 to 10,000,000 primary probes interacting with one or more molecular targets. In some embodiments, the method comprises 20 to 10,000,000 primary probes interacting with one or more molecular targets. In some embodiments, the method comprises 30 to 10,000,000 primary probes interacting with one or more molecular targets. In some embodiments, the method comprises 50 to 10,000,000 primary probes interacting with one or more molecular targets. In some embodiments, the method comprises 75 to 100 primary probes interacting with one or more molecular targets.

In some embodiments, the primary probe has a molecular target sequence (site), wherein the molecular target sequence interacts with the molecular target. In some embodiments, the molecular target sequence is between 2 and 1000 nucleotides in length. In some embodiments, the primary probe comprises 1, 2, 3, 4, 5, 6, 7, 8, 9, or 10 molecular target sites. In some embodiments, the molecular target sequences are the same. In some embodiments, the molecular target sequences are different and can interact with different molecular targets.

In some embodiments, the primary probe comprises one or more first binding sites, wherein the first binding site interacts with the primary probe anchor on a secondary probe. In some embodiments, the first binding site is between 2 and 500 nucleotides in length. In some embodiments, the primary probe comprises 1, 2, 3, 4, 5, 6, 7, 8, 9, or 10 first binding sites. In some embodiments, the primary probe comprises between 1 to 100 first binding sites. In some embodiments, the first binding sites are the same and interact with the same primary probe anchor on different secondary probes. In some embodiments, the first binding sites are different, and interact with different probe anchors on different secondary probes.

In some embodiments, each primary probe in the plurality of primary probes comprises a molecular target binding site, one or more first binding sites, wherein each first binding site is capable of interacting with a primary probe anchor on one of the secondary probes in the plurality of secondary probes, and optionally one or more readout probe binding sites.

In some embodiments, the method forms primary probes that hybridize with the molecular targets. In some embodiments, each primary probe in the one or more primary probes is formed by contacting a molecular target in the cell with an anchor probe that hybridizes to the molecular target. In some embodiments, each primary probe in the one or more primary probes is formed by contacting a molecular target in the cell with a primary anchoring fragment that hybridizes to the molecular target. In some embodiments, the method comprises linking the anchor probe to the primary anchoring fragment to form a molecular target anchor. In some embodiments, the method comprises contacting the molecular target anchor with a primary reading fragment, wherein the primary reading fragment hybridizes to the molecular target and/or the primary anchoring fragment. In some embodiments, the method comprises linking the primary reading fragment and the primary anchoring fragment to form the primary probe. In some embodiments, the method comprises linking the primary probe to cellular components. In some embodiments, the method comprises linking the primary probe to substrates. In some embodiments, the method comprises linking the primary probe to functionalized cell components or gel matrices.

In some embodiments, the method comprises that for each primary probe in the plurality of primary probes, the primary probe comprises a molecular target binding site, and one or more first binding sites, wherein each first binding site interacts with a primary probe anchor on one of the secondary probes in the plurality of secondary probes.

### Secondary Probes

In some embodiments, the method comprises one or more secondary probes interacting with one or more primary probes. In some embodiments, the method comprises one or more secondary probes binding to one or more primary probes. In some embodiments, the method comprises one or more secondary probes hybridizing with the one or more primary probes.

In some embodiments, the method comprises one to 1000 secondary probes interacting with one or more primary probes. In some embodiments, the method comprises 2 to 1000 secondary probes interacting with one or more primary probes. In some embodiments, the method comprises 5 to 1000 secondary probes interacting with one or more primary probes. In some embodiments, the method comprises 10 to 1000 secondary probes interacting with one or more primary probes. In some embodiments, the method comprises 15 to 1000 secondary probes interacting with one or more primary probes. In some embodiments, the method comprises 20 to 1000 secondary probes interacting with one or more primary probes. In some embodiments, the method comprises 30 to 1000 secondary probes interacting with one or more primary probes. In some embodiments, the method comprises 50 to 1000 secondary probes interacting with one or more primary probes. In some embodiments, the method comprises 75 to 1000 secondary probes interacting with one or more primary probes.

In some embodiments, the secondary probe is an oligonucleotide. In some embodiments, the secondary probe is between 2 and 10000 nucleotides in length.

In some embodiments, the secondary probe comprises one or more binding sites, wherein the second binding site interacts with the secondary probe anchor on the tertiary probe. In some embodiments, the second binding site is between 2 and 500 nucleotides in length. In some embodiments, the secondary probe comprises 1, 2, 3, 4, 5, 6, 7, 8, 9, or 10 second binding sites. In some embodiments, the secondary probe comprises between 1 to 100 second binding sites. In some embodiments, the second binding sites are the same and interact with the same tertiary probe anchor. In some embodiments, the second binding sites are different and interact with different probe anchors on different tertiary probes.

In some embodiments, the secondary probe has a primary probe anchor, wherein the primary probe anchor interacts with the first binding site. In some embodiments, the primary probe anchor is between 2 and 500 nucleotides in length.

In some embodiments, each secondary probe or secondary reading fragments in the plurality of secondary probes comprises the primary probe anchor, wherein the primary probe anchor comprises a complementary sequence that interacts with one of the first binding sites on one of the primary probes in the plurality of primary probes. In some embodiments, each secondary probe or secondary reading fragments in the plurality of secondary probes comprises one or more second binding sites, wherein each second binding site that interacts with a secondary probe anchor on one of the tertiary probes in the plurality of tertiary probes. In some embodiments, each secondary probe or secondary reading fragments in the plurality of secondary probes comprises, optionally, one or more readout probe binding sites, wherein each readout probe binding site comprises a complementary sequence that interacts with one of the readout probes in the plurality of readout probes.

In some embodiments, each secondary probe comprises one or more secondary reading fragments and one or more secondary anchoring fragments. In some embodiments, each secondary probe and/or secondary reading fragment comprises one or more second binding sites, wherein each second binding site interacts with a tertiary probe, tertiary reading fragment, and/or tertiary anchor fragment. In some embodiments, each secondary probe or secondary reading fragments in the plurality of secondary probes comprises, optionally, one or more readout probe binding sites, wherein each readout probe binding site comprises a complementary sequence that interacts with one of the readout probes in the plurality of readout probes.

In some embodiments, each secondary probe in the plurality of secondary probes comprises the primary probe anchor, wherein the primary probe anchor comprises a complementary sequence that interacts with one of the first binding sites on one of the primary probes in the plurality of primary probes. In certain embodiments, the secondary probe comprises one or more second binding sites, wherein each second binding site that interacts with a secondary probe anchor on one of the tertiary probes in the plurality of tertiary probes. In certain embodiments, the secondary probe comprise, optionally, one or more readout probe binding sites, wherein each readout probe binding site comprises a complementary sequence that interacts with one of the readout probes in the plurality of readout probes.

In some embodiments, the method comprises secondary probes hybridizing to the quaternary, tertiary, and/or primary probes.

### Tertiary Probes

In some embodiments, the method comprises one or more tertiary probes interacting with one or more secondary probes. In some embodiments, the method comprises one or more tertiary probes binding to the one or more secondary probes. In some embodiments, the method comprises one or more tertiary probes hybridizing with one or more secondary probes.

In some embodiments, the method comprises one to 100 tertiary probes interacting with one or more secondary probes. In some embodiments, the method comprises 2 to 100 tertiary probes interacting with one or more secondary probes. In some embodiments, the method comprises 5 to 100 tertiary probes interacting with one or more secondary probes. In some embodiments, the method comprises 10 to 100 tertiary probes interacting with one or more secondary probes. In some embodiments, the method comprises 15 to 100 tertiary probes interacting with one or more secondary probes. In some embodiments, the method comprises 20 to 100 tertiary probes interacting with one or more secondary probes. In some embodiments, the method comprises 30 to 100 tertiary probes interacting with one or more secondary probes. In some embodiments, the method comprises 50 to 100 tertiary probes interacting with one or more secondary probes. In some embodiments, the method comprises 75 to 100 tertiary probes interacting with one or more secondary probes.

In some embodiments, the tertiary probe is an oligonucleotide. In some embodiments, the tertiary probe is between 2 and 10000 nucleotides in length.

In some embodiments, the tertiary probe comprises a third binding site, wherein the third binding site interacts with the tertiary probe anchor on a quaternary probe. In some embodiments, the third binding site is between 2 and 500 nucleotides in length. In some embodiments, the tertiary probe comprises 1, 2, 3, 4, 5, 6, 7, 8, 9, or 10 third binding sites. In some embodiments, the tertiary probe comprises between 1 to 100 third binding sites. In some embodiments, the third binding sites are the same and interact with the same quaternary probe anchor. In some embodiments, the third binding sites are different and can interact with different probe anchors on the quaternary probes.

In some embodiments, the tertiary probe has a secondary probe anchor, wherein the secondary probe anchor interacts with the second binding site on the secondary probe. In some embodiments, the secondary probe anchor is between 2 and 500 nucleotides in length.

In some embodiments, each tertiary probe or tertiary reading fragment in the plurality of tertiary probes comprises: the secondary probe anchor, wherein each secondary probe anchor comprises a complementary sequence that interacts with one of the second binding sites on one of the secondary probes in the plurality of secondary probes. In some embodiments, each tertiary probe or tertiary reading fragment in the plurality of tertiary probes comprises one or more third binding sites, wherein each third binding site comprises a complementary sequence to one of the quaternary probe anchors on one of the quaternary probes in the plurality of quaternary probes. In some embodiments, each tertiary probe or tertiary reading fragment in the plurality of tertiary probes comprises, optionally, one or more readout probe binding sites, wherein each readout probe binding site comprises a complementary sequence that interacts with one of the readout probes in the plurality of readout probes.

In some embodiments, the method comprises tertiary probe in the plurality of tertiary probes comprises the secondary probe anchor, wherein each secondary probe anchor comprises a complementary sequence that interacts with one of the second binding sites on one of the secondary probes in the plurality of secondary probes. In certain embodiments, the tertiary probe comprises one or more third binding sites, wherein each third binding site comprises a complementary sequence to one of the quaternary probe anchors on one of the quaternary probes in the plurality of quaternary probes. In certain embodiments, the tertiary probe comprises optionally, one or more readout probe binding sites, wherein each readout probe binding site comprises a complementary sequence that interacts with one of the readout probes in the plurality of readout probes.

In some embodiments, each tertiary probe comprises one or more tertiary reading fragments and/or tertiary anchoring fragments. In some embodiments, each tertiary probe or tertiary reading fragment in the plurality of tertiary probes comprises one or more second binding sites, wherein each second binding site interacts with a secondary probe, secondary reading fragment, and/or secondary anchor fragment. In some embodiments, each tertiary probe in the plurality of tertiary probes comprises one or more second binding sites, wherein each second binding site interacts with a quaternary probe, quaternary reading fragment, and/or quaternary anchor fragment. In some embodiments, each tertiary probe or tertiary reading fragments in the plurality of tertiary probes comprises, optionally, one or more readout probe binding sites, wherein each readout probe binding site comprises a complementary sequence that interacts with one of the readout probes in the plurality of readout probes.

In some embodiments, the method comprises tertiary probes hybridizing to the quaternary, secondary, and/or primary probes.

### Quaternary Probes

In some embodiments, the method comprises one or more quaternary probes interacting with one or more secondary and/or tertiary probes. In some embodiments, the method comprises one or more quaternary probes binding to the one or more secondary and/or tertiary probes. In some embodiments, the method comprises one or more quaternary probes hybridizing with the one or more secondary and/or tertiary probes.

In some embodiments, the method comprises one to 100 quaternary probes interacting with one or more secondary and/or tertiary probes. In some embodiments, the method comprises 2 to 100 quaternary probes interacting with one or more secondary and/or tertiary probes. In some embodiments, the method comprises 5 to 100 quaternary probes interacting with one or more secondary and/or tertiary probes. In some embodiments, the method comprises 10 to 100 quaternary probes interacting with one or more secondary and/or tertiary probes. In some embodiments, the method comprises 15 to 100 quaternary probes interacting with one or more secondary and/or tertiary probes. In some embodiments, the method comprises 20 to 100 quaternary probes interacting with one or more secondary and/or tertiary probes. In some embodiments, the method comprises 30 to 100 quaternary probes interacting with one or more secondary and/or tertiary probes. In some embodiments, the method comprises 50 to 100 quaternary probes interacting with one or more secondary and/or tertiary probes. In some embodiments, the method comprises 75 to 100 quaternary probes interacting with one or more secondary and/or tertiary probes.

In some embodiments, the quaternary probe is an oligonucleotide. In some embodiments, the quaternary probe is between 2 and 10000 nucleotides in length. In some embodiments, the quaternary probe comprises the second binding sites or third binding sites of any of the previous embodiments. In some embodiments, the quaternary probe comprises between 1 to 100 second binding sites. In some embodiments, the quaternary probe comprises between 1 to 100 third binding sites.

In some embodiments, the third binding site is between 2 and 500 nucleotides in length. In some embodiments, the quaternary probe comprises the third binding site, wherein the third binding site interacts with the primary anchor probe. In some embodiments, secondary probe comprises 1, 2, 3, 4, 5, 6, 7, 8, 9, or 10 third binding sites. In some embodiments, secondary probe comprises 1 to 100 third binding sites. In some embodiments, the third binding sites are the same and can interact with the same probe anchor. In some embodiments, the third binding sites are different and can interact with different probe anchors. In some embodiments, the quaternary probe comprises 1 to 100 third binding sites.

In some embodiments, the quaternary probe has a tertiary probe anchor, wherein the tertiary probe anchor interacts with the third binding site on the tertiary probe. In some embodiments, the tertiary probe anchor is between 2 and 500 nucleotides in length.

In some embodiments, each quaternary probe or quaternary reading fragment in the plurality of quaternary probes comprises: the tertiary probe anchor, wherein each tertiary probe anchor comprises a complementary sequence that interacts with one of the third binding sites on one of the tertiary probes in the plurality of tertiary probes. In some embodiments, each quaternary probe or quaternary reading fragment in the plurality of quaternary probes comprises one or more second binding sites and/or third binding sites. In some embodiments, each quaternary probe or quaternary reading fragment in the plurality of quaternary probes, comprises optionally, one or more readout probe binding sites, wherein each readout probe binding site comprises a complementary sequence that interacts with one of the readout probes in the plurality of readout probes.

In some embodiments, the method comprises each quaternary probe in the plurality of quaternary probes comprises the tertiary probe anchor, wherein each tertiary probe anchor comprises a complementary sequence that interacts with one of the third binding sites on one of the tertiary probes in the plurality of tertiary probes. In certain embodiments, each quaternary probe comprises one or more second binding sites and/or third binding sites. In certain embodiments, each quaternary probe comprises optionally, one or more readout probe binding sites, wherein each readout probe binding site comprises a complementary sequence that interacts with one of the readout probes in the plurality of readout probes.

In some embodiments, each quaternary probe comprises one or more quaternary reading fragments and/or quaternary anchoring fragments. In some embodiments, each quaternary probe or quaternary reading fragments in the plurality of quaternary probes comprises one or more second binding sites, wherein each second binding site interacts with a secondary probe, secondary reading fragment, and/or secondary anchor fragment. In some embodiments, each quaternary probe in the plurality of quaternary probes comprises one or more second binding sites, wherein each second binding site interacts with a tertiary probe, tertiary reading fragment, and/or tertiary anchor fragment. In some embodiments, each quaternary probe or quaternary reading fragment in the plurality of quaternary probes comprises, optionally, one or more readout probe binding sites, wherein each readout probe binding site comprises a complementary sequence that interacts with one of the readout probes in the plurality of readout probes.

In some embodiments, the method comprises quaternary probes hybridizing to the tertiary, secondary, and/or primary probes.

### Readout Probe Binding Sites

In some embodiments, readout probes interact with readout binding sites on the primary, secondary, tertiary, and/or quaternary probes. In some embodiments, there are 1, 2, 3, 4, 5, 6, 7, 8, 9, or 10 readout binding sites on the probes of any of the previous embodiments.

In some embodiments, the readout probe binding sites are between 2 and 500 nucleotides in length. In some embodiments, the primary, secondary, tertiary, and/or quaternary probes comprise 1 to 100 readout probe binding sites.

### Anchor Probes

In some embodiments, the anchor probes are selected from oligonucleotides, proteins, antibodies, protein-oligonucleotide conjugates, antibody oligonucleotide-conjugates, peptides, small molecules, and any combinations thereof that can interact with molecular targets.

In some embodiments, the anchor probes are oligonucleotides between 2 and 500 nucleotides in length. In some embodiments, the anchoring fragments are oligonucleotides between 2 and 500 nucleotides in length.

### Reading Fragments and Anchoring Fragments

In some embodiments, the methods comprise primary, secondary, tertiary, and/or quaternary reading fragments.

In some embodiments, the reading fragments are selected from oligonucleotides, proteins, antibodies, protein-oligonucleotide conjugates, antibody oligonucleotide-conjugates, peptides, small molecules, and any combinations thereof that can interact with molecular targets.

In some embodiments, the reading fragments are selected from oligonucleotides, proteins, antibodies, protein-oligonucleotide conjugates, antibody oligonucleotide-conjugates, peptides, small molecules, and any combinations thereof that can interact with reading fragments and/or reading anchors.

In some embodiments, the reading fragments are oligonucleotides between 2 and 500 nucleotides in length. In some embodiments, the reading fragments are oligonucleotides between 2 and 500 nucleotides in length.

In some embodiments, the method comprises the primary reading fragment interacting with the molecular target.

In some embodiments, the method comprises the secondary reading fragment interacting with the primary reading fragment.

In some embodiments, the method comprises the tertiary reading fragment interacting with the secondary reading fragment.

In some embodiments, the method comprises the secondary reading fragments hybridizing to the quaternary, tertiary, and/or primary fragments.

In some embodiments, the method comprises the tertiary reading fragments hybridizing to the quaternary, secondary, and/or primary reading fragments.

In some embodiments, the method comprises the quaternary reading fragments hybridizing to the tertiary, secondary, and/or primary reading fragments.

In some embodiments, the primary reading fragments and/or primary anchoring fragments hybridize to the one or more molecular targets.

In some embodiments, the secondary reading fragments and/or secondary anchoring fragments hybridize to the one or more primary probes.

In some embodiments, the tertiary reading fragments and/or tertiary anchoring fragments hybridize to the secondary probes.

In some embodiments, the methods comprise primary, secondary, tertiary, and/or quaternary anchoring fragments.

In some embodiments, the anchoring fragments are selected from oligonucleotides, proteins, antibodies, protein-oligonucleotide conjugates, antibody oligonucleotide-conjugates, peptides, small molecules, and any combinations thereof that can interact with molecular targets.

In some embodiments, the anchoring fragments are oligonucleotides between 2 and 500 nucleotides in length.

In some embodiments, the anchoring fragments are also known as bridge probes.

In some embodiments, the method comprises secondary reading fragments and/or secondary anchoring fragments hybridizing to the one or more primary probes. In some embodiments, the method comprises tertiary reading fragments and/or tertiary anchoring fragments hybridizing to the secondary probes.

In some embodiments, the method comprises two or more tertiary reading fragments interacting with two or more secondary reading fragments, which in turn interact with two or more primary probes that interact with different molecular targets. In some embodiments, the method comprises two or more quaternary reading fragments interacting with the two or more primary probes that interact with different molecular targets. In some embodiments, the method comprises two or more quaternary reading fragments interacting with two or more tertiary reading fragments, which in turn interact with two or more secondary reading fragments, which in turn interact with two or more primary probes that interact with different molecular targets.

In some embodiments, the method comprises one or more secondary reading fragments interacting with each secondary anchoring fragment that are the same. In some embodiments, the method comprises one or more secondary reading fragments interacting with each secondary anchoring fragment are different. In some embodiments, the method comprises the one or more tertiary reading fragments interacting with each tertiary anchoring fragment are the same. In some embodiments, the method comprises the one or more tertiary reading fragments interacting with each tertiary anchoring fragment are different.

In some embodiments, the method comprises that each secondary anchoring fragment interacting with two or more secondary reading fragments, comprises one or more complementary sequences that interact with each of the secondary reading fragments.

In some embodiments, the method comprises that each tertiary anchoring fragment interacting with two or more tertiary reading fragments, comprises one or more complementary sequences that interact with each of the tertiary reading fragments.

In some embodiments, the method comprises the one or more quaternary reading fragments interacting with each quaternary anchoring fragment are the same. In some embodiments, the method comprises the one or more quaternary reading fragments interacting with each quaternary anchoring fragment are different. In some embodiments, the method comprise each quaternary anchoring fragment interacting with two or more quaternary reading fragments comprises one or more complementary sequences that interact with each of the quaternary reading fragments.

In some embodiments, the method comprises one or more secondary reading fragments interacting with one of the two or more primary probes and one or more secondary anchoring fragments interacting with a different primary probe. In some embodiments, the method comprises one or more secondary reading fragments interacting with the one or more secondary anchoring fragments. In some embodiments, the method comprises two or more tertiary reading fragments interacting with two or more primary probes that interact with different molecular targets.

In some embodiments, the method of any of the previous embodiments, comprises one to 1000 secondary reading fragments interacting with one or more primary probes. In some embodiments, the method of any of the previous embodiments, comprises two to 1000 secondary reading fragments interacting with one or more primary probes. In some embodiments, the method of any of the previous embodiments, comprises three to 1000 secondary reading fragments interacting with one or more primary probes. In some embodiments, the method of any of the previous embodiments, comprises four to 1000 secondary reading fragments interacting with one or more primary probes. In some embodiments, the method of any of the previous embodiments, comprises five to 1000 secondary reading fragments interacting with one or more primary probes. In some embodiments, the method of any of the previous embodiments, comprises 10 to 1000 secondary reading fragments interacting with one or more primary probes. In some embodiments, the method of any of the previous embodiments, comprises 20 to 1000 secondary reading fragments interacting with one or more primary probes. In some embodiments, the method of any of the previous embodiments, comprises 30 to 1000 secondary reading fragments interacting with one or more primary probes. In some embodiments, the method of any of the previous embodiments, comprises 40 to 1000 secondary reading fragments interacting with one or more primary probes. In some embodiments, the method of any of the previous embodiments, comprises 50 to 1000 secondary reading fragments interacting with one or more primary probes. In some embodiments, the method of any of the previous embodiments, comprises 75 to 1000 secondary reading fragments interacting with one or more primary probes. In some embodiments, the method of any of the previous embodiments, comprises 90 to 1000 secondary reading fragments interacting with one or more primary probes.

In some embodiments, the method of any of the previous embodiments, comprises one to 100 secondary anchoring fragments interacting with one or more primary probes. In some embodiments, the method of any previous embodiments, comprises one to 10 secondary anchoring fragments interacting with one or more primary probes. In some embodiments, the method of any of the previous embodiments, comprises two to 100 secondary anchoring fragments interacting with one or more primary probes. In some embodiments, the method of any of the previous embodiments, comprises three to 100 secondary anchoring fragments interacting with one or more primary probes. In some embodiments, the method of any of the previous embodiments, comprises four to 100 secondary anchoring fragments interacting with one or more primary probes. In some embodiments, the method of any of the previous embodiments, comprises five to 100 secondary anchoring fragments interacting with one or more primary probes. In some embodiments, the method of any of the previous embodiments, comprises 10 to 100 secondary anchoring fragments interacting with one or more primary probes. In some embodiments, the method of any of the previous embodiments, comprises 20 to 100 secondary anchoring fragments interacting with one or more primary probes. In some embodiments, the method of any of the previous embodiments, comprises 30 to 100 secondary anchoring fragments interacting with one or more primary probes. In some embodiments, the method of any of the previous embodiments, comprises 40 to 100 secondary anchoring fragments interacting with one or more primary probes. In some embodiments, the method of any of the previous embodiments, comprises 50 to 100 secondary anchoring fragments interacting with one or more primary probes. In some embodiments, the method of any of the previous embodiments, comprises 75 to 100 secondary anchoring fragments interacting with one or more primary probes. In some embodiments, the method of any of the previous embodiments, comprises 90 to 100 secondary anchoring fragments interacting with one or more primary probes.

In some embodiments, the method of any of the previous embodiments, comprises one to 1000 tertiary reading fragments interacting with one or more secondary reading fragments and/or anchoring fragments. In some embodiments, the method of any of the previous embodiments, comprises two to 1000 tertiary reading fragments interacting with one or more secondary reading fragments and/or anchoring fragments. In some embodiments, the method of any of the previous embodiments, comprises three to 1000 tertiary reading fragments interacting with one or more secondary reading fragments and/or anchoring fragments. In some embodiments, the method of any of the previous embodiments, comprises four to 1000 tertiary reading fragments interacting with one or more secondary reading fragments and/or anchoring fragments. In some embodiments, the method of any of the previous embodiments, comprises five to 1000 tertiary reading fragments interacting with one or more secondary reading fragments and/or anchoring fragments. In some embodiments, the method of any of the previous embodiments, comprises 10 to 1000 tertiary reading fragments interacting with one or more secondary reading fragments and/or anchoring fragments. In some embodiments, the method of any of the previous embodiments, comprises 20 to 1000 tertiary reading fragments interacting with one or more secondary reading fragments and/or anchoring fragments. In some embodiments, the method of any of the previous embodiments, comprises 30 to 1000 tertiary reading fragments interacting with one or more secondary reading fragments and/or anchoring fragments. In some embodiments, the method of any of the previous embodiments, comprises 40 to 1000 tertiary reading fragments interacting with one or more secondary reading fragments and/or anchoring fragments. In some embodiments, the method of any of the previous embodiments, comprises 50 to 1000 tertiary reading fragments interacting with one or more secondary reading fragments and/or anchoring fragments. In some embodiments, the method of any of the previous embodiments, comprises 75 to 1000 tertiary reading fragments interacting with one or more secondary reading fragments and/or anchoring fragments. In some embodiments, the method of any of the previous embodiments, comprises 90 to 1000 tertiary reading fragments interacting with one or more secondary reading fragments and/or anchoring fragments.

In some embodiments, the method of any of the previous embodiments, comprises one to 100 tertiary anchoring fragments interacting with one or more secondary reading fragments and/or anchoring fragments. In some embodiments, the method of any of the previous embodiments, comprises one to 10 tertiary anchoring fragments interacting with one or more secondary reading fragments and/or anchoring fragments. In some embodiments, the method of any of the previous embodiments, comprises two to 100 tertiary anchoring fragments interacting with one or more secondary reading fragments and/or anchoring fragments. In some embodiments, the method of any of the previous embodiments, comprises three to 100 tertiary anchoring fragments interacting with one or more secondary reading fragments and/or anchoring fragments. In some embodiments, the method of any of the previous embodiments, comprises four to 100 tertiary anchoring fragments interacting with one or more secondary reading fragments and/or anchoring fragments. In some embodiments, the method of any of the previous embodiments, comprises five to 100 tertiary anchoring fragments interacting with one or more secondary reading fragments and/or anchoring fragments. In some embodiments, the method of any of the previous embodiments, comprises 10 to 100 tertiary anchoring fragments interacting with one or more secondary reading fragments and/or anchoring fragments. In some embodiments, the method of any of the previous embodiments, comprises 20 to 100 tertiary anchoring fragments interacting with one or more secondary reading fragments and/or anchoring fragments. In some embodiments, the method of any of the previous embodiments, comprises 30 to 100 tertiary anchoring fragments interacting with one or more secondary reading fragments and/or anchoring fragments. In some embodiments, the method of any of the previous embodiments, comprises 40 to 100 tertiary anchoring fragments interacting with one or more secondary reading fragments and/or anchoring fragments. In some embodiments, the method of any of the previous embodiments, comprises 50 to 100 tertiary anchoring fragments interacting with one or more secondary reading fragments and/or anchoring fragments. In some embodiments, the method of any of the previous embodiments, comprises 75 to 100 tertiary anchoring fragments interacting with one or more secondary reading fragments and/or anchoring fragments. In some embodiments, the method of any of the previous embodiments, comprises 90 to 100 tertiary anchoring fragments interacting with one or more secondary reading fragments and/or anchoring fragments.

In some embodiments, the method of any of the previous embodiments, comprises one to 1000 quaternary reading fragments interacting with one or more tertiary reading fragments and/or anchoring fragments. In some embodiments, the method of any of the previous embodiments, comprises two to 1000 quaternary reading fragments interacting with one or more tertiary reading fragments and/or anchoring fragments. In some embodiments, the method of any of the previous embodiments, comprises three to 1000 quaternary reading fragments interacting with one or more tertiary reading fragments and/or anchoring fragments. In some embodiments, the method of any of the previous embodiments, comprises four to 1000 quaternary reading fragments interacting with one or more tertiary reading fragments and/or anchoring fragments. In some embodiments, the method of any of the previous embodiments, comprises five to 1000 quaternary reading fragments interacting with one or more tertiary reading fragments and/or anchoring fragments. In some embodiments, the method of any of the previous embodiments, comprises 10 to 1000 quaternary reading fragments interacting with one or more tertiary reading fragments and/or anchoring fragments. In some embodiments, the method of any of the previous embodiments, comprises 20 to 1000 quaternary reading fragments interacting with one or more tertiary reading fragments and/or anchoring fragments. In some embodiments, the method of any of the previous embodiments, comprises 30 to 1000 quaternary reading fragments interacting with one or more tertiary reading fragments and/or anchoring fragments. In some embodiments, the method of any of the previous embodiments, comprises 40 to 1000 quaternary reading fragments interacting with one or more tertiary reading fragments and/or anchoring fragments. In some embodiments, the method of any of the previous embodiments, comprises 50 to 1000 quaternary reading fragments interacting with one or more tertiary reading fragments and/or anchoring fragments. In some embodiments, the method of any of the previous embodiments, comprises 75 to 1000 quaternary reading fragments interacting with one or more tertiary reading fragments and/or anchoring fragments. In some embodiments, the method of any of the previous embodiments, comprises 90 to 1000 quaternary reading fragments interacting with one or more tertiary reading fragments and/or anchoring fragments.

In some embodiments, the method of any of the previous embodiments, comprises one to 100 quaternary anchoring fragments interacting with one or more tertiary reading fragments and/or anchoring fragments. In some embodiments, the method of any previous embodiments, comprises one to 10 tertiary anchoring fragments interacting with with one or more tertiary reading fragments and/or anchoring fragments. In some embodiments, the method of any of the previous embodiments, comprises two to 100 quaternary anchoring fragments interacting with one or more tertiary reading fragments and/or anchoring fragments. In some embodiments, the method of any of the previous embodiments, comprises three to 100 quaternary anchoring fragments interacting with one or more tertiary reading fragments and/or anchoring fragments. In some embodiments, the method of any of the previous embodiments, comprises four to 100 quaternary anchoring fragments interacting with one or more tertiary reading fragments and/or anchoring fragments. In some embodiments, the method of any of the previous embodiments, comprises five to 100 quaternary anchoring fragments interacting with one or more tertiary reading fragments and/or anchoring fragments. In some embodiments, the method of any of the previous embodiments, comprises 10 to 100 quaternary anchoring fragments interacting with one or more tertiary reading fragments and/or anchoring fragments. In some embodiments, the method of any of the previous embodiments, comprises 20 to 100 quaternary anchoring fragments interacting with one or more tertiary reading fragments and/or anchoring fragments. In some embodiments, the method of any of the previous embodiments, comprises 30 to 100 quaternary anchoring fragments interacting with one or more tertiary reading fragments and/or anchoring fragments. In some embodiments, the method of any of the previous embodiments, comprises 40 to 100 quaternary anchoring fragments interacting with one or more tertiary reading fragments and/or anchoring fragments. In some embodiments, the method of any of the previous embodiments, comprises 50 to 100 quaternary anchoring fragments interacting with one or more tertiary reading fragments and/or anchoring fragments. In some embodiments, the method of any of the previous embodiments, comprises 75 to 100 quaternary anchoring fragments interacting with one or more tertiary reading fragments and/or anchoring fragments. In some embodiments, the method of any of the previous embodiments, comprises 90 to 100 quaternary anchoring fragments interacting with one or more tertiary reading fragments and/or anchoring fragments.

### Bridge Complexes

In some embodiments, the method comprises forming bridge complexes.

In some embodiments, the bridge complexes comprise secondary, tertiary, and/or quaternary bridge complexes.

In some embodiments, a secondary bridge complex comprises one or more secondary anchoring fragments interacting with two or more secondary reading fragments, which in turn interact with two or more primary probes. In certain embodiments, the two or more secondary reading fragments are the same. In certain embodiments, the two or more secondary reading fragments are different. In certain embodiments, the two or more primary probes are the same. In certain embodiments, the two or more primary probes are different.

In some embodiments, a tertiary bridge complex comprises one or more tertiary anchoring fragments interacting with two or more tertiary reading fragments, which in turn interact with two or more secondary reading fragments, which in turn interacts with two or more primary probes. In certain embodiments, the two or more tertiary reading fragments are the same. In certain embodiments, the two or more tertiary reading fragments are different. In certain embodiments, the two or more secondary reading fragments are the same. In certain embodiments, the two or more secondary reading fragments are different. In certain embodiments, the two or more primary probes are the same. In certain embodiments, the two or more primary probes are different. In certain embodiments, the tertiary bridge complex optionally comprises a secondary anchoring fragment that interacts with the two or more secondary reading fragments.

In some embodiments, a quaternary bridge complex comprises one or more quaternary anchoring fragments interacting with two or more quaternary reading fragments, which in turn interact with two or more tertiary reading fragments, which in turn interact with two or more secondary reading fragments, which in turn interacts with two or more primary probes. In certain embodiments, the two or more quaternary reading fragments are the same. In certain embodiments, the two or more quaternary reading fragments are different. In certain embodiments, the two or more tertiary reading fragments are the same. In certain embodiments, the two or more tertiary reading fragments are different. In certain embodiments, the two or more secondary reading fragments are the same. In certain embodiments, the two or more secondary reading fragments are different. In certain embodiments, the two or more primary probes are the same. In certain embodiments, the two or more primary probes are different. In certain embodiments, the quaternary bridge complex optionally comprises a tertiary anchoring fragment that interacts with the two or more tertiary reading fragments. In certain embodiments, the tertiary bridge complex optionally comprises a secondary anchoring fragment that interacts with the two or more secondary reading fragments.

In certain embodiments, each of the secondary, tertiary, and/or quaternary bridge complexes comprise primary probes interacting with the same molecular target. In certain embodiments, each of the secondary, tertiary, and/or quaternary bridge complexes comprise primary probes interacting with different molecular targets.

### Proximity

In some embodiments, the method provides a measure of proximity between one molecular target to another. In certain embodiments, the interaction of the two or more secondary reading fragments with the two or more primary probes is a measure of proximity between one molecular target to another. In certain embodiments, the interaction of the two or more tertiary reading fragments with the two or more primary probes is a measure of proximity between one molecular target to another. In certain embodiments, the interaction of the two or more quaternary reading fragments with the two or more primary probes is a measure of proximity between one molecular target to another. In certain embodiments, the interaction of the one or more secondary reading fragments with the one or more secondary anchoring fragments is a measure of proximity between one molecular target to another. In certain embodiments, the interaction of the one or more secondary reading fragments with the one or more secondary anchoring fragments is a measure of proximity between one primary probe to another.

In certain embodiments, the interaction of the one or more secondary reading fragments with another secondary reading fragment and/or other secondary reading fragments is a measure of proximity between one molecular target to another. In certain embodiments, the interaction of the one or more secondary reading fragments with one or more secondary reading fragments and/or other secondary reading fragments is a measure of proximity between one molecular target to another.

In certain embodiments, the interaction of the one or more secondary reading fragments with another secondary reading fragment and/or other secondary reading fragments is a measure of proximity between one primary probe to another. In certain embodiments, the interaction of the one or more secondary reading fragments with one or more secondary reading fragments and/or other secondary reading fragments is a measure of proximity between one primary probe to another.

In some embodiments, the proximity between one molecular target and another is between 0.1 to 100 nanometers. In certain embodiments, the proximity between one molecular target and another is between 1 to 100 nanometers. In certain embodiments, the proximity between one molecular target and another is between 5 to 100 nanometers. In certain embodiments, the proximity between one molecular target and another is between 10 to 100 nanometers. In certain embodiments, the proximity between one molecular target and another is between 20 to 100 nanometers. In certain embodiments, the proximity between one molecular target and another is between 30 to 100 nanometers. In certain embodiments, the proximity between one molecular target and another is between 50 to 100 nanometers. In certain embodiments, the proximity between one molecular target and another is between 75 to 100 nanometers. In certain embodiments, the proximity between one molecular target and another is between 1 to 10 nanometers. In certain embodiments, the proximity between one molecular target and another is between 1 to 20 nanometers.

### Stabilization

In some embodiments, the methods of any of the previous embodiments further comprise stabilizing the primary probes, secondary probes, tertiary probes, and/or quaternary probes during or after each contacting step. In certain embodiments, the primary probes, secondary probes, tertiary probes, and/or quaternary probes are stabilized by ligating the probes to themselves, or to cellular components. In certain embodiments, the primary probes, secondary probes, tertiary probes, and/or quaternary probes are stabilized by linking the probes to themselves, or to cellular components. In certain embodiments, the primary probes, primary anchors, secondary reading fragments, secondary anchoring fragments, tertiary reading fragments, tertiary anchoring fragments, quaternary reading fragments, and/or quaternary anchoring fragments are stabilized by crosslinking, such as photo-crosslinking or chemical crosslinking.

### Antibodies

In some embodiments, the method comprises reading and anchoring antibodies. In some embodiments, the reading antibodies and anchoring antibodies bind to the protein at binding sites on the protein. In some embodiments, the reading antibodies and anchoring probes bind to the molecular target at binding sites on the molecular target.

### Linking, Photo-Crosslinkers, and Covalent Crosslinkers

In some embodiments, the methods comprise linking fragments, probes, cellular components, and/or molecular targets.

In some embodiments, the one or more primary probes, secondary anchoring fragments, secondary reading fragments, secondary probes, tertiary reading fragments, tertiary anchoring fragments, tertiary probes, quaternary reading fragments, quaternary anchoring fragments, and/or quaternary probes comprise 5′ cross-linkable moieties, 3′ cross-linkable moieties, cross-linkable moieties between the 5′ and 3′ ends, or combinations thereof. In some embodiments, the one or more primary probes, secondary anchoring fragments, secondary reading fragments, tertiary reading fragments, tertiary anchoring fragments, quaternary reading fragments, and/or quaternary anchoring fragments comprise 5′ cross-linkable moieties, 3′ cross-linkable moieties, cross-linkable moieties between the 5′ and 3′ ends, or combinations thereof. In some embodiments, the one or more anchor probes, primary anchoring fragments, primary reading fragments, oligonucleotides, antibody oligonucleotide conjugates, secondary reading fragments, and/or tertiary reading fragments comprise 5′ and/or 3′ cross-linkable moieties.

In some embodiments, linking one more cellular components to primary probes, secondary anchoring fragments, secondary reading fragments, secondary probes, tertiary reading fragments, tertiary anchoring fragments, tertiary probes, quaternary reading fragments, quaternary anchoring fragments, and/or quaternary probes aids retention during stringent washes. In certain embodiments, stringent washes reduce amplification from nonspecific signals and/or nonspecific interactions of the primary probes, secondary anchoring fragments, secondary reading fragments, secondary probes, tertiary reading fragments, tertiary anchoring fragments, tertiary probes, quaternary reading fragments, quaternary anchoring fragments, and/or quaternary probes. In certain embodiments, linking of cellular components to primary probes, secondary anchoring fragments, secondary reading fragments, secondary probes, tertiary reading fragments, tertiary anchoring fragments, tertiary probes, quaternary reading fragments, quaternary anchoring fragments, and/or quaternary probes increases the amplification yield of specific interactions and decreases nonspecific interactions.

### Linking to Cellular Components:

In some embodiments, the method comprises linking reading fragments to cellular components. In certain embodiments, the method comprises linking primary, secondary, and/or tertiary reading fragments to cellular components. In some embodiments, the method further comprises linking the secondary, tertiary, and/or quaternary probes to cellular components.

In some embodiments, the method further comprises linking the anchor probe to the cellular component. In certain embodiments, anchor probes react with the cellular component. In certain embodiments, the reaction forms a covalent bond between the anchor probe and the cellular component.

### Linking to Probes:

In some embodiments, the method further comprises linking the secondary probe, tertiary, and/or quaternary probes with the anchor probe.

In some embodiments, the method further comprises contacting the cell with the anchor probe before, during or after any of the steps.

In some embodiments, the method further comprises linking one or more primary reading fragments, primary anchor fragments, secondary reading fragments, secondary anchoring fragments, secondary probes, tertiary reading fragments, tertiary anchoring fragments, and/or tertiary probes to one or more cellular components at any of the steps of the method.

In some embodiments, the method further comprises linking one or more primary probes, primary reading fragments, primary anchor fragments, secondary reading fragments, secondary anchoring fragments, secondary probes, tertiary reading fragments, tertiary anchoring fragments, and/or tertiary probes to one or more functionalized cellular component and/or cellular components of a gel matrix any of the steps of the previous embodiments.

In some embodiments, the method further comprises linking the one or more primary probes, primary reading fragments, primary anchor fragments, secondary reading fragments, secondary anchoring fragments, secondary probes, tertiary reading fragments, tertiary anchoring fragments, tertiary probes, quaternary reading fragments, quaternary anchoring fragments, and/or quaternary probes to the substrate at any of the steps of the method. In some embodiments, the method further comprises linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to one or more functionalized cellular component and/or cellular components of a gel matrix any of the steps of the previous embodiments.

The method of any of the previous embodiments, further comprising linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to a substrate, wherein the substrate is the same substrate that the primary probes interact with, at any of the previous steps of the method.

The method of any of the previous embodiments, further comprising linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to a substrate, wherein the substrate is a different substrate that the substrate interacting with the primary probes, at any of the previous steps of the method.

In some embodiments, the method further comprises linking the one or more primary probes to the one or more secondary reading fragments, secondary anchoring fragments, tertiary reading fragments, and/or tertiary anchoring fragments at any of the steps of the method.

In some embodiments, the method further comprises linking the one or more secondary reading fragments to the one or more secondary anchoring fragments, secondary probes, tertiary reading fragments, tertiary anchoring fragments, and/or tertiary probes at any of steps of the method.

In some embodiments, the method further comprises linking the one or more secondary anchoring fragments to the secondary probes, the one or more tertiary reading fragments, tertiary anchoring fragments, and/or tertiary probes at any of steps of the method.

In some embodiments, the method further comprises linking the secondary probes to the one or more tertiary reading fragments and/or tertiary anchoring fragments at any of steps of the method.

In some embodiments, the method further comprises linking one or more tertiary reading fragments to the one or more tertiary anchoring fragments at any of steps of the method.

In some embodiments, the secondary reading fragment is linked to a secondary anchoring fragment. In certain embodiments, the secondary reading fragments linked to the secondary anchoring fragments, each interact with the same primary probe. In certain embodiments, the secondary reading fragment linked to the secondary anchoring fragment directly interacts with the same primary probe. In certain embodiments, the secondary reading fragment linked to a secondary anchoring fragment indirectly interacts with the same primary probe.

In some embodiments, the tertiary reading fragment is linked to a tertiary anchoring fragment. In certain embodiments, the tertiary reading fragments linked to the tertiary anchoring fragments, each interact with the same secondary probe. In certain embodiments, the tertiary reading fragment linked to a tertiary anchoring fragment directly interacts with the same secondary probe. In certain embodiments, the tertiary reading fragment linked to a tertiary anchoring fragment indirectly interact with the same secondary probe.

Photo-crosslinking:

In some embodiments, the method comprises using photo-crosslinkers and covalent crosslinkers.

In some embodiments, the one or more primary probes, secondary anchoring fragments, secondary reading fragments, tertiary reading fragments, tertiary anchoring fragments, quaternary reading fragments, and/or quaternary anchoring fragments comprise photo-crosslinkers.

In some embodiments, the cross-linkable moieties are photo-cross-linkable molecules. In certain embodiments, the photo-crosslinkers are selected from diazirines, benzophenones, aryl-azides, thiol-enes systems, crosslinkers configured for cycloaddition reactions, and combinations thereof. In certain embodiments, the cycloaddition reactions are Diels-Alder reaction. In certain embodiments, the method comprises photo-crosslinkers selected from diazirines, benzophenones, aryl-azides, and any combination thereof.

In some embodiments, the method comprises photo-crosslinking one or more anchor probes, primary anchoring fragments, primary reading fragments, oligonucleotides, antibody oligonucleotide conjugates, secondary reading fragments, secondary anchor fragments, tertiary anchor fragments and/or tertiary reading fragments to one or more cellular components.

In some embodiments, one or more anchor probes, primary anchoring fragments, primary reading fragments, oligonucleotides, antibody oligonucleotide conjugates, secondary reading fragments, and/or tertiary reading fragments comprise photo-crosslinkers.

In some embodiments, the cross-linkable moieties are photo-cross-linkable molecules. In some embodiments, the method comprises photo-crosslinking the anchor probe to the primary anchoring fragment. In some embodiments, the method comprises photo-crosslinking the primary reading fragment to the primary anchoring fragment. In some embodiments, the method comprises photo-crosslinking the secondary reading fragment to the secondary anchoring fragment. In some embodiments, the method comprises photo-crosslinking the tertiary reading fragment to the tertiary anchoring fragment. In some embodiments, the method comprises photo-crosslinking an anchor probe to the primary, secondary, and/or tertiary anchoring fragments. In some embodiments, the method comprises photo-crosslinking one or more anchor probes, primary anchoring fragments, primary reading fragments, oligonucleotides, antibody oligonucleotide conjugates, secondary anchor fragments, and/or tertiary anchor fragments to each other at any of the steps in the previous embodiments of the methods. In some embodiments, the method comprises photo-crosslinking one or more anchor probes, primary anchoring fragments, primary reading fragments, oligonucleotides, antibody oligonucleotide conjugates, secondary anchor fragments, and/or tertiary anchor fragments to one or more cellular components at any of the steps in the previous embodiments of the methods.

In some embodiments, the method comprises photo-crosslinking the anchor probe, primary anchoring fragment, and/or primary reading fragment, to one or more cellular components at any of the steps in the previous embodiments of the methods.

### Linking:

In some embodiments, the method comprises linking one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to one or more cellular components at any of the steps of the previous embodiments. In some embodiments, the method comprises linking one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to one or more functionalized cellular component and/or cellular components of a gel matrix any of the steps of the previous embodiments.

In some embodiments, the method comprises covalently linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to one or more cellular components at any of the steps of the previous embodiments. In some embodiments, the method comprises covalently linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to one or more functionalized cellular component and/or cellular components of a gel matrix any of the steps of the previous embodiments.

In some embodiments, the method comprises covalently linking the anchor probe to the primary anchoring fragment. In some embodiments, the method comprises covalently linking the primary reading fragment to the primary anchoring fragment. In some embodiments, the method comprises covalently linking an anchor probe to the primary, secondary, and/or tertiary anchoring fragments.

In some embodiments, the method comprises covalently linking one or more anchor probes, primary anchoring fragments, primary reading fragments, oligonucleotides, antibody oligonucleotide conjugates, secondary reading fragments, and/or tertiary reading fragments to one or more cellular components at any of the steps in the previous embodiments of the methods.

In some embodiments, the method comprises covalently linking the anchor probe, primary anchoring fragment, and/or primary reading fragment to one or more cellular components at any of the steps in the previous embodiments of the methods.

In some embodiments, the method comprises linking one or more anchor probes, primary anchoring fragments, primary reading fragments, oligonucleotides, antibody oligonucleotide conjugates, secondary reading fragments, and/or tertiary reading fragments to one or more cellular components at any of the steps in the previous embodiments of the methods.

In some embodiments, the method comprises linking the anchor probe, primary anchoring fragment, and/or primary reading fragment to one or more cellular components at any of the steps in the previous embodiments of the methods.

In some embodiments, the method further comprises linking the one or more primary probes to the one or more secondary reading fragments, secondary anchoring fragments, tertiary reading fragments, and/or tertiary anchoring fragments at any of the steps of the method.

In some embodiments, the method further comprises linking the one or more secondary reading fragments to the one or more secondary anchoring fragments, secondary probes, tertiary reading fragments, tertiary anchoring fragments, tertiary probes, quaternary reading fragments, quaternary anchoring fragments, and/or quaternary probes at any of the steps in the method.

In some embodiments, the method comprises further comprising linking the one or more secondary anchoring fragments to the secondary probes, the one or more tertiary reading fragments, tertiary anchoring fragments, tertiary probes, quaternary reading fragments, quaternary anchoring fragments, and/or quaternary probes at any of the steps in the method.

Chemical reaction:
In some embodiments, the method comprises linking one or more anchor probes, primary anchoring fragments, primary reading fragments, oligonucleotides, secondary reading fragments, and/or tertiary reading fragments to one or more cellular components by using a chemical reaction. In some embodiments, the chemical reaction comprises copper-catalyzed azide-alkyne cycloaddition reaction (CuAAC), Staudinger ligation, copper-free azide-alkyne cycloaddition, tetrazine ligation, tetrazole ligation, oxime ligation, condensation of acids with amines, and thiol-ene reactions.

In some embodiments, the samples can be functionalized with reactive groups. In some embodiments, the functionalize comprises reactions with NHS-azide and NHS-alkyne.

Enzyme reaction:
In some embodiments, the method comprises linking one or more anchor probes, primary anchoring fragments, primary reading fragments, oligonucleotides, secondary reading fragments, and/or tertiary reading fragments to one or more cellular components by using an enzymatic reaction. In certain embodiments, the enzymes comprise ligases, a polymerase, reverse transcriptases, nucleases, biotin ligases, or transferases.

### Detectably Labelled Probes

In some embodiments, the method comprises locating molecular targets by using detectably labelled probes. In some embodiments, the detectably labeled probes are readout probes. In some embodiments, the method comprises one or more readout probes that hybridize to the primary, secondary, and/or tertiary probes.

In some embodiments, the one or more readout probes comprise one or more detectable moieties.

In some embodiments, the one or more readout probes are sequencing probes.

In some embodiments, the one or more readout probes comprise one or more amplifiable sequences. In certain embodiments, the one or more amplifiable sequences hybridize to detectably labelled probes. In certain embodiments, the one or more amplifiable sequences are amplifiable by hybridization chain reaction (HCR). In certain embodiments, the one or more amplifiable sequences are amplifiable by polymerase chain reaction.

In certain embodiments, the one or more readout probes are detected by detecting one or more detectable moieties on the one or more readout probes. In certain embodiments, the one or more readout probes are detected by amplifying the one or more amplifiable sequences.

In some embodiments, the detectably labelled probe is selected from proteins, modified proteins, RNA, oligonucleotides, antibodies, antibody fragments, and combinations thereof. In some embodiments, the method comprises the detectably labelled readout probes interacting with the secondary and/or tertiary reading fragments by binding or hybridizing to readout probe binding sites. In some embodiments, the detectably labelled probe further comprises a detectably moiety. In some embodiments, the detectably moiety is a fluorophore.

In some embodiments, the method comprises readout probes hybridizing to the secondary, tertiary, or quaternary reading fragments by hybridizing to readout probe binding sites on the secondary, tertiary, or quaternary fragments.

In some embodiments, the detectably labelled probe comprises an oligonucleotide with a detectably moiety.

In some embodiments, the detectably labelled probe comprises oligonucleotides that are at least 5 nucleotides long. In some embodiments, the detectably labelled probe comprises oligonucleotides that are at least 6 nucleotides long. In some embodiments, the detectably labelled probe comprises oligonucleotides that are at least 7 nucleotides long. In some embodiments, the detectably labelled probe comprises oligonucleotides that are at least 8 nucleotides long. In some embodiments, the detectably labelled probe comprises oligonucleotides that are at least 9 nucleotides long. In some embodiments, the detectably labelled probe comprises oligonucleotides that are at least 10 nucleotides long. In some embodiments, the detectably labelled probe comprises oligonucleotides that are at least 11 nucleotides long. In some embodiments, the detectably labelled probe comprises oligonucleotides that are at least 12 nucleotides long. In some embodiments, the detectably labelled probe comprises oligonucleotides that are at least 13 nucleotides long. In some embodiments, the detectably labelled probe comprises oligonucleotides that are at least 14 nucleotides long. In some embodiments, the detectably labelled probe comprises oligonucleotides that are at least 15 nucleotides long. In some embodiments, the detectably labelled probe comprises oligonucleotides that are at least 16 nucleotides long. In some embodiments, the detectably labelled probe comprises oligonucleotides that are at least 17 nucleotides long. In some embodiments, the detectably labelled probe comprises oligonucleotides that are at least 18 nucleotides long. In some embodiments, the detectably labelled probe comprises oligonucleotides that are at least 19 nucleotides long. In some embodiments, the detectably labelled probe comprises oligonucleotides that are at least 20 nucleotides long. In some embodiments, the detectably labelled probe comprises oligonucleotides that are at least 21 nucleotides long. In some embodiments, the detectably labelled probe comprises oligonucleotides that are at least 22 nucleotides long. In some embodiments, the detectably labelled probe comprises oligonucleotides that are at least 23 nucleotides long. In some embodiments, the detectably labelled probe comprises oligonucleotides that are at least 24 nucleotides long. In some embodiments, the detectably labelled probe comprises oligonucleotides that are at least 25 nucleotides long. In some embodiments, the detectably labelled probe comprises oligonucleotides that are at least 26 nucleotides long. In some embodiments, the detectably labelled probe comprises oligonucleotides that are at least 27 nucleotides long. In some embodiments, the detectably labelled probe comprises oligonucleotides that are at least 28 nucleotides long. In some embodiments, the detectably labelled probe comprises oligonucleotides that are at least 29 nucleotides long. In some embodiments, the detectably labelled probe comprises oligonucleotides that are at least 30 nucleotides long. In some embodiments, the detectably labelled probes of any of the previous embodiments comprise oligonucleotides that are less than 35, 40, 45, 50, 100 nucleotides in length.

In some embodiments, the detectably labelled probe comprises a sequence that is complementary to the secondary and/or tertiary probes. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the detectably labelled probes comprise oligonucleotides with the same sequence. In some embodiments, the detectably labelled probes comprise oligonucleotides with different sequences.

In some embodiments, the one or more readout probes of any of the previous embodiments comprise one or more detectable moieties.

In some embodiments, the one or more readout probes comprise one or more amplifiable sequences. In certain embodiments, the one or more amplifiable sequences hybridize to detectably labelled probes. In certain embodiments, the one or more readout probes are detected by detecting one or more detectable moieties on the one or more readout probes. In certain embodiments, the one or more readout probes are detected by amplifying the one or more amplifiable sequences.

In some embodiments, the method comprises one or more readout probes are sequencing probes.

In some embodiments, the method comprises readout probes that are FRET pairs. In some embodiment one or more readout probes are fluorescent donors. In certain embodiments, one or more readout probes are fluorescent acceptors. In certain embodiments, these FRET pairs may be used as discussed in the MAPPING and FORSTER RESONANCE ENERGY TRANSFER (FRET) section below.

In some embodiments, the method comprises detecting the sequencing probes by sequencing-by-ligation. In some embodiments, the method comprises detecting the sequencing probes by sequencing-by synthesis.

In some embodiments, the method comprises detecting the one or more primary probes interacting with the substrate by sequencing-by-synthesis of the secondary reading fragments, secondary anchoring probes, and/or secondary probes, tertiary reading fragments, tertiary anchoring probes, and/or tertiary probes, or any oligonucleotides hybridized directly or indirectly with the primary probes. In certain embodiments, the oligonucleotides are hybridized indirectly through intermediate oligonucleotides.

In some embodiments, the method comprises readout probes hybridizing to secondary, tertiary, or quaternary probes. In certain embodiments, the readout probe hybridizes to readout probe binding sites on the secondary, tertiary, or quaternary probes.

### Stacks of Probes

In some embodiments, the method comprises primary probes, secondary probes, tertiary probes, quaternary probes, and/or readout probes forming stacks of probes.

In some embodiments, a first primary probe interacts with one or more secondary probes, tertiary probes, quaternary probes, and/or readout probes to form a first stack of probes. In some embodiments, a second primary probe interacts with different secondary probes, tertiary probes, quaternary probes, and/or readout probes to form a second stack of probes.

In some embodiments, a first secondary probe interacts with one or more primary probes, tertiary probes, quaternary probes, and/or readout probes to form a first stack of probes. In some embodiments, a second secondary probe interacts with different primary probes, tertiary probes, quaternary probes, and/or readout probes to form a second stack of probes.

In some embodiments, optionally, a first tertiary probe interacts with one or more primary probes, secondary probes, quaternary probes, and/or readout probes to form a first stack of probes. In some embodiments, optionally, a second tertiary probe interacts with different primary probes, secondary probes, quaternary probes, and/or readout probes to form a second stack of probes.

In some embodiments, optionally, a first quaternary probe interacts with one or more primary probes, secondary probes, tertiary probes, and/or readout probes to form a first stack of probes. In some embodiments, optionally, a second quaternary probe interacts with different primary probes, secondary probes, tertiary probes, and/or readout probes to form a second stack of probes.

In some embodiments, optionally, a first readout probe interacts with one or more primary probes, secondary probes, tertiary probes, and/or quaternary probes to form a first stack of probes. In some embodiments, optionally, a second readout probe interacts with different primary probes, secondary probes, tertiary probes, and/or quaternary probes to form a second stack of probes.

In some embodiments, the method comprises each of the one or more primary probes interacting with a stack of probes comprising one or more secondary probes, one or more tertiary probes, optionally, one or more quaternary probes, and/or one or more readout probes to generate a signal indicating the position of each of the one or more primary probes interacting with the substrate.

In some embodiments, the method comprises each of the one or more primary probes interacting with a stack of probes, wherein each of the one or more primary probes interacts with one of the one or more secondary probes. In certain embodiments, the method comprises each of the one or more secondary probes interacting with a stack of probes, wherein each of the one or more secondary probes interacts with one of the one or more tertiary probes. In certain embodiments, the method comprises each of the one or more tertiary probes interacting with a stack of probes, wherein each of the one or more tertiary probes interacts with one of the one or more quaternary probes. In certain embodiments, the method comprises each of the one or more primary, secondary, tertiary, and/or quaternary probes interacting with one or more readout probes to generate a signal indicating the position of each of the one or more primary probes interacting with the substrate.

### Fluorophores

In some embodiments, the oligonucleotides or proteins, used to map the cellular components in any of the previous embodiments, comprise one or more fluorophores. In some embodiments, the fluorophores used to perform mapping of cellular components can be any technique deemed suitable by a person of skill in art.

In certain embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red). In certain embodiments, the fluorophores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein. In certain embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750. In certain embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the label comprises a nanomaterial. In some embodiments, the label is a nanoparticle. In some embodiments, the label is or comprises a quantum dot. In some embodiments, the fluorophore is a quantum dot. In some embodiments, the label comprises a quantum dot. In some embodiments, the label is or comprises a gold nanoparticle. In some embodiments, the label is a gold nanoparticle. In some embodiments, the label comprises a gold nanoparticle.

In some embodiments, the term “label” in any of the previous embodiments, may be synonymous with fluorophore.

In some embodiments, the methods of any of the previous embodiments comprise one or more probes linked with a plurality of labels that produce different signals.

### Mapping AND Förster Resonance Energy Transfer (FRET)

In some embodiments, the method further comprises a method to map the interaction of molecular targets in a cell. In some embodiments, the method comprises detecting one or more readout probes to detect one or more primary probes interacting with the substrate and thereby detecting the positions of the one or more primary probes interacting with the substrate.

In some embodiments, the method further comprises distinguishing differences in the positions between the one or more primary probes interacting with the substrate.

In certain embodiments, the interaction is the proximity of one molecular target to another in a cell. In certain embodiments, the interaction is the proximity of one molecular target to another on a substrate.

In some embodiments, the method comprises measuring the proximity by Förster Resonance Energy Transfer (FRET). In certain embodiments, the proximity is measured by Electron Transfer (ET). In certain embodiments, the interaction of molecular targets is detected with single molecule accuracy. In certain embodiments, the method comprises imaging the cell so that the proximity between the first molecular target and the second molecular target is detected from the interaction of the fluorescent donor and fluorescent acceptor.

In some embodiments, a method is described comprising steps of contacting a cell comprising molecular targets with a plurality of primary probes, each primary probe interacting with a molecular target, so that the plurality comprises a first primary probe that interacts with a first molecular target in the cell, and a second primary probe that interacts with a second molecular target in the cell. In some embodiments, the method comprises forming, by contacting secondary probes to the plurality of primary probes, a first probe complex on the first primary probe and a second probe complex on the second primary probe. In some embodiments, the method comprises contacting a plurality of detectably labelled readout probes to binding sites on the first and second probe complexes. In some embodiments the plurality of detectably labelled readout probes comprises a first readout probe that interacts with the first probe complex, wherein the first readout probe comprises a fluorescence donor, and a second readout probe that interacts with the second probe complex, wherein the second readout probe comprises a fluorescence acceptor. In some embodiments, the method comprises imaging the cell so that the proximity between the first molecular target and the second molecular target is detected from the interaction of the fluorescent donor and fluorescent acceptor.

In some embodiments, a first probe complex comprises secondary probes interacting with a first primary probe, wherein the first primary probe interacts with a first molecular target. In some embodiments, a second probe complex comprises secondary probes interacting with a second primary probe, wherein the second primary probe interacts with a second molecular target different than the first.

In some embodiments, the method comprises contacting a cell comprising molecular targets with a plurality of primary probes, each primary probe interacting with a molecular target. In certain embodiments, the plurality of primary probes comprises a first primary probe that interacts with a first molecular target in the cell and a second primary probe that interacts with a second molecular target in the cell. In some embodiments, the method comprises contacting the primary probes with a plurality of secondary probes, wherein the plurality of secondary probes comprises a first secondary probe that interacts with the first primary probe and a second secondary probe that interacts with the second primary probe. In some embodiments, the method comprises optionally, contacting the secondary probes with a plurality of tertiary probes, wherein the plurality of tertiary probes comprises a first tertiary probe that interacts with the first secondary probe, and a second tertiary probe that interacts with the second secondary probe. In some embodiments, the method comprises optionally, contacting the tertiary probes with a plurality of quaternary probes after, wherein the plurality of quaternary probes comprises a first quaternary probe that interacts with the first tertiary probe or the first secondary probe; and a second quaternary probe that interacts with the second tertiary probe or the second secondary probe. In some embodiments, the method comprises optionally, repeating any of the previous contacting steps, wherein the secondary, tertiary, or quaternary probes comprise readout probe binding sites. In some embodiments, the method comprises contacting the secondary, tertiary, or quaternary probes with a plurality of readout probes capable of detection. In some embodiments, the plurality of readout probes comprises a first readout probe that interacts with the first secondary probes, tertiary probes, or quaternary probes. In some embodiments, the first readout probe comprises a moiety that is a fluorescent donor and a second readout probe that interacts with the second secondary probes, tertiary probes, or quaternary probes. In some embodiments, the second readout probe comprises a moiety that is a fluorescent acceptor. In some embodiments, the method comprises imaging the cell so that the proximity between the first molecular target and the second molecular target is detected from the interaction of the fluorescent donor and fluorescent acceptor.

In some embodiments, method of any of the previous embodiments comprises contacting the primary probes with a plurality of secondary probes, wherein the plurality of secondary probes comprises a first secondary probe that interacts with the first primary probe. In certain embodiments, the plurality of secondary probes comprises a second secondary probe that interacts with the second primary probe.

In some embodiments, method of any of the previous embodiments comprises forming the first and second probe complexes by contacting the secondary probes with a plurality of tertiary probes, wherein the plurality of tertiary probes comprises a first tertiary probe that interacts with the first secondary probe. In certain embodiments, the plurality of tertiary probes comprises a second tertiary probe that interacts with the second secondary probe.

In some embodiments, method of any of the previous embodiments comprises contacting the tertiary probes with a plurality of quaternary probes, wherein the plurality of quaternary probes comprises a first quaternary probe that interacts with the first tertiary probe. In certain embodiments, the plurality of quaternary probes comprises and a second quaternary probe that interacts with the second tertiary probe.

In some embodiments, the method further comprises repeating the contacting and imaging steps, each time with a new plurality of detectably labeled readout probes, wherein in each new plurality each readout probe targets a different secondary probe, tertiary probe, and/or quaternary probe, and wherein each readout probe differs in their fluorescent donor and fluorescent acceptor.

In some embodiments, method of any of the previous embodiments comprises optionally, repeating contacting the first and second probe complexes with a new plurality of primary, secondary, tertiary, and/or quaternary probes. In some embodiments, the contacting steps are repeated 1, 2, 3, 4, 5, 6, 7, 8, 9, 10, 11, 12, 13, 14, 15, 20, 25, 30, 35, 40, 45, 50, or 100 times.

### Washes

In some embodiments, the method comprises washing the cell to remove unhybridized and/or unlinked primary anchoring fragments, anchor probes, and/or primary reading fragments at any of the previous steps. In certain embodiments, the washing comprises contacting the cell with stripping reagents, wash buffers, photobleaching, chemical bleaching, and any combinations thereof.

Certain techniques for removing probes are known in the art. See, for example:
a. International PCT Patent Application No. PCT/US2014/036258, filed Apr. 30, 2014, and titled MULTIPLEX LABELING OF MOLECULES BY SEQUENTIAL HYBRIDIZATION BARCODING, the entire contents of which are herein incorporated by reference in its entirety for all purposes.
b. International PCT Patent Application No. PCT/US2018/064616, filed Dec. 7, 2018, and titled MULTIPLEX LABELING OF MOLECULES Publication Classification BY SEQUENTIAL HYBRIDIZATION BARCODING WITH RAPID SWITCHING AND REHYBRIDIZATION OF PROBES, the entire contents of which are herein incorporated by reference in its entirety for all purposes.
c. International PCT Patent Application No. PCT/US2017/044994, filed Aug. 1, 2017, and titled SEQUENTIAL PROBING OF MOLECULAR TARGETS BASED ON PSEUDO—COLOR BARCODES WITH EMBEDDED ERROR CORRECTION MECHANISM, the entire contents of which are herein incorporated by reference in its entirety for all purposes.
d. International PCT Patent Application No. PCT/US2022/024494, filed Apr. 12, 2022, and titled HIGH-RESOLUTION WHOLE GENOME IMAGING BY NUCLEIC ACID LOCUS AND BLOCK CODING, the entire contents of which are herein incorporated by reference in its entirety for all purposes.
e. International PCT Patent Application No. PCT/US2022/032736, filed Jun. 8, 2022, and titled RATIOMETRIC SYMBOLS AND SEQUENTIAL CODING FOR MULTIPLEXED FISH, the entire contents of which are herein incorporated by reference in its entirety for all purposes.
f. International PCT Patent Application No. PCT/US2022/053995, filed Dec. 23, 2022, and titled SUPPRESSION OF NON-SPECIFIC SIGNALS BY EXONUCLEASES IN FISH EXPERIMENT, the entire contents of which are herein incorporated by reference in its entirety for all purposes.
g. International PCT Patent Application No. PCT/US2022/051737, filed Dec. 2, 2022, and titled METHOD OF MAPPING SPATIAL DISTRIBUTIONS OF CELLULAR COMPONENTS, the entire contents of which are herein incorporated by reference in its entirety for all purposes.
h. International PCT Patent Application No. PCT/US2022/017757, filed Feb. 24, 2022, and titled MULTIPLEXING OF EXPERIMENTAL CONDITIONS AND SAMPLES IN SPATIAL GENOMICS, the entire contents of which are herein incorporated by reference in its entirety for all purposes.
i. International PCT Patent Application No. PCT/US2025/015646, filed Feb. 12, 2025, and titled SUPER-RESOLVED OBJECT DETECTION WITH SPATIAL GENOMICS, the entire contents of which are herein incorporated by reference in its entirety for all purposes.

### EXAMPLES

### Example 1

### Method Description:

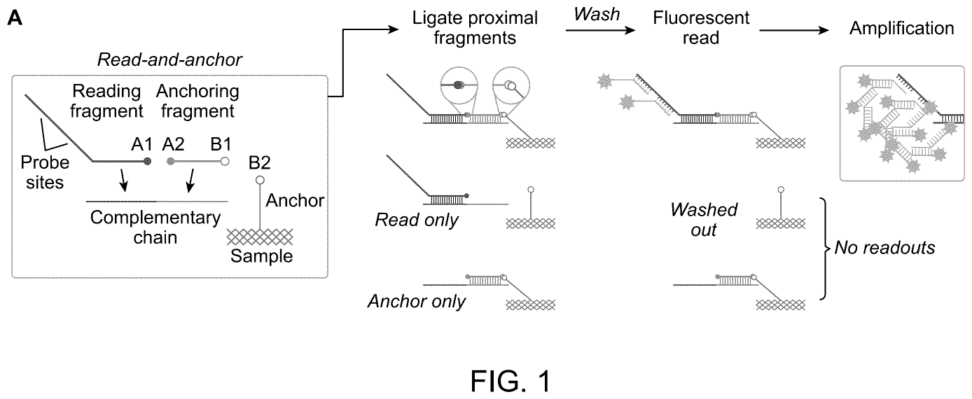

The methods described in this application, called the “Read-and-Anchor Split Probe” strategy, separate probe hybridization and stabilization both in process and in terms of reagents. In this approach (FIG. 1), the anchoring functional group and readout (or amplifier) binding site are strategically separated. The ligation reaction is designed to occur only under conditions where both fragments bind to their respective templates and are spatially proximal. Alternatively, the anchoring segment is extended from the read agent in a target-specific fashion. The methods described enable a higher level of specificity in signal amplification.

### DEFINITIONS OF A1, A2, B1, AND B2

“A” represents a process used to connect the “read probe” with an anchoring moiety.

“B” refers to a process for anchoring hybridized probes onto cellular or tissue samples.

A and B are distinct processes with orthogonal reaction conditions. A1, A2, B1, and B2 denote the key functional groups that enable these processes.

When the ligation process (A) is enzyme-mediated, A1 and A2, as well as B1 and B2, may be free-end phosphate or hydroxyl groups. Possible enzymes include T3 DNA ligase, T4 DNA ligase, T4 RNA ligase, SplintR ligase. Alternatively, when the ligation is chemically driven, these groups may include alkyne, azide, carboxylic acid, amine, or amine-derived functionalities.

Every primary probe is split into two components. The reading fragment contains a readout or amplifier binding region, a target binding region, and a reaction handle A1 for process A. The anchoring fragment contains a target binding region, reaction handle A2 for process A, and reaction handle B1 for process B. Additionally, the cell or tissue sample can be pre-modified with reaction handle B2 for process B.

In one implementation, using an in vivo imaging assay, the sample was first incubated with the split primary probes under optimized conditions to achieve target-specific hybridization. Subsequently, a ligation solution containing the necessary reagents or enzymes for process A was introduced. Upon completing process A, crosslinking reagents were applied to facilitate process B. The sample was then washed harshly to eliminate non-covalently bound probes, such as with concentrated formamide, thereby reducing non-specific binding.

The anchored primary probes were either directly visualized using fluorescent readout probes or subjected to further amplification through additional binding of amplifiers, to enhance signal sensitivity and clarity.

In another implementation, the primary probe containing the amplification binding region was extended in a target-dependent fashion. This was accomplished using reverse transcriptase for RNA targets or polymerase for DNA targets.

### Combined with Amplification (Amplifier Probes Splitting)

Utilizing split secondary and tertiary probes, the method was further amplified. These probes consisted of two components: The amplifying fragment contained a binding site for secondary or tertiary probes, a hybridization domain, and a reaction handle (A1) for process A. The anchoring fragment included a component that can be bound or extended from the primary probe, a reaction handle (A2) for process A, and a reaction handle (B1) for process B.

In this modified approach, process A facilitated the ligation of probe fragments, while process B ensured the anchored attachment of ligated probes onto the sample. By iteratively performing the hybridization-ligation-anchoring cycle, this enhanced protocol achieved greater signal sensitivity and clarity compared to the original amplification. These improvements allowed for more precise visualization and quantification of transcriptional activity in complex biological systems.

### Mapping Antibodies with Enhanced Specificity

To achieve higher specificity in antibody-based detection, a dual-antibody system was used in which two antibodies bind simultaneously to the same target protein. The “reading antibody” was linked to the “anchoring antibody” such that only when both antibodies were bound, the reading antibody was retained in the cell through the anchoring antibody. For example, “reading antibody” was labeled with an oligonucleotide containing an amplification or readout sequence and ligation handle A1, while the “anchoring antibody” is labeled with ligation handles A2 and Bl. The sample itself was modified with ligation handle B2. After antibody binding, a bridge oligonucleotide was introduced, designed to hybridize with both oligonucleotide tags on the antibodies. Ligation reagents were then added to enable covalent bonding. Ideally, the ligation reaction occurs only when both antibody tags are bound to the bridge oligonucleotide. Following this, process B was facilitated under specific reaction conditions, and a stringent wash step was performed to remove any components not covalently attached to the sample. Another implementation was used where only the reading antibody contained a barcode that could be detected, whereas the “anchoring antibody” contained a moiety that could react with the reading antibody. This approach enhanced amplification specificity and significantly reduced noise from non-specific binding, resulting in a more accurate detection system.

### Sample Preparation

When B2 was carboxyl acid, the sample surface was blocked with N-(propionyloxy) succinimide (100 mM in 10% DMSO/PBS, 1 h for 3 times). When B2 was an amine, the sample surface was blocked with 1-Ethyl-3-(3-dimethylaminopropyl)carbodiimide (abbreviated as EDC) solution, followed by tris(hydroxymethyl)aminomethane (Tris) solution. Otherwise, the sample was modified with NHS ester, which contains the B2 functional group.

### Imaging

The readout probes were modified with Alexa 647 Cy3b, and Alexa 488.

The anchoring reaction was performed using carboxyl-amine condensation. EDC/NHS reaction condition is from immobilizing protein receptors in SPR assay. The NIH/3T3 cell sample preparation follows the workflow described in the referenced literature. See Eng, C H. et al.

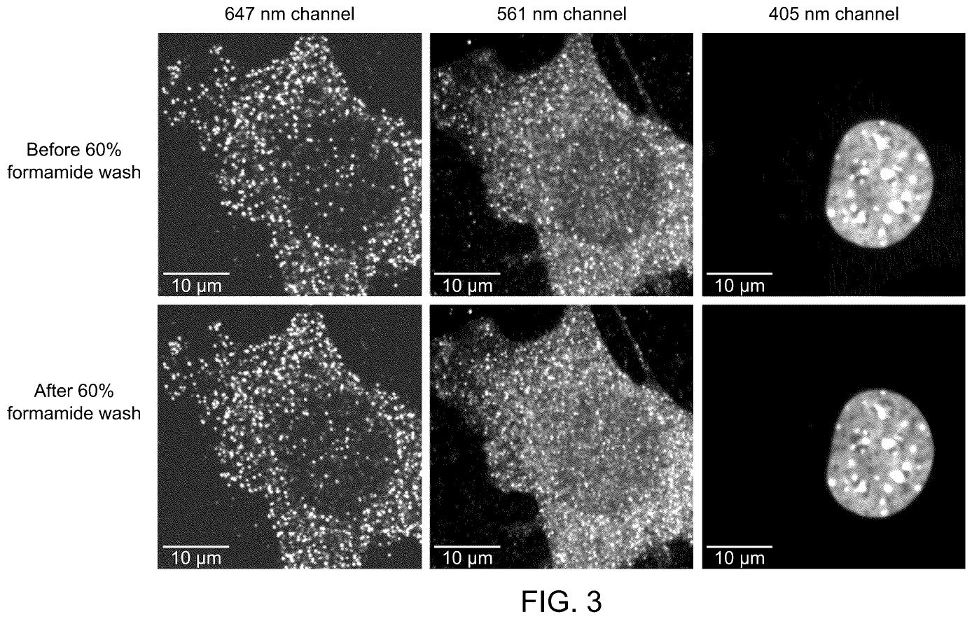

Single-molecule fluorescence in situ hybridization (smFISH) was performed to assess the efficiency of the carboxyl-amine condensation reaction in this method. The NIH/3T3 cell sample was incubated overnight with 24 eukaryotic translation elongation factor 2 (eef2) targeted primary probes, followed by a 1-hour incubation at 37 °C with amine-modified secondary probes (sec_R1_R7 and sec_R3_R9), where “R1, R3” correspond to primary binding sites, and R7 and R9 correspond to tertiary probe binding sequences. The samples were then treated with an EDC solution (0.1 M MES, 0.5 M NaCl, 200 mM EDC HCl, pH 6.0) at 37 °C for 1 hour to facilitate covalent anchoring of the probes to their targets. After the reaction, the samples were washed with PBST (1×PBS, 0.1% Triton X-100) and incubated for 30 minutes, followed by washes with 15% formamide washing buffer and 4×SSC. Readout probes specific for sec_R1_R7 (labeled with AF647) and sec_R3_R9 (labeled with Cy3b), along with DAPI, were hybridized to the samples, and imaging was performed before and after a 60% formamide wash. The 60% formamide wash was used to selectively remove non-covalently bound probes. The comparison of images before and after washing (FIG. 3) revealed that most of the fluorescence signals were retained post-wash, indicating that the ligation reaction achieved high efficiency in covalently anchoring the probes.

Alternatively, the EDC/NHS solution (0.1 M MES, 0.5 M NaCl, 200 mM EDC HCl, 50 mM NHS, pH 6.0) can also be used.

### In Vivo Investigation of “Read-and-Anchor Split Probe” Strategy

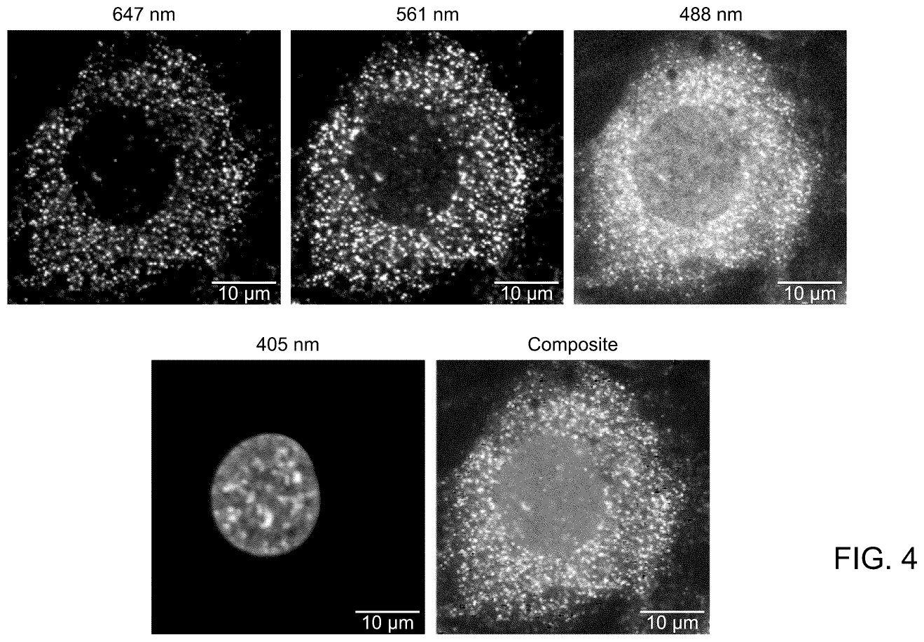

An assay utilizing six primary probes targeting eef2 mRNA, with process A catalyzed by T3 ligase, was conducted. The NIH/3T3 cell sample preparation followed the workflow outlined in the referenced literature. Id. Amplification was performed in 12 sequential rounds, each involving hybridization of secondary or tertiary amplifiers for 1 hour, followed by sequential incubation with enzyme buffer and hybridization of the enzyme and azide-modified anchoring fragments for 30 minutes. The enzyme and anchoring fragments were then flowed into the system and incubated at 37 °C for an additional 30 minutes to enable covalent anchoring. Results (FIG. 4) demonstrated effective signal amplification in the split amplifier group, with signals showing stronger co-localization effects between 647 and 561 nm channels (two separate pairs of amplifiers) and reduced nonspecific nuclear signal (as detected by 405 nm laser excitation) compared to the control group. One additional amplifier pair in the 488 channel did not amplify.

### Azide-Alkyne Cycloaddition and 4-Benzoylbenzoic Acid (BBA)-Mediated Photo-Crosslinking

Copper(I)-catalyzed Azide-Alkyne Cycloaddition (CuAAC; Process A) and 4-benzoylbenzoic acid (BBA)-mediated photo-crosslinking (Process B) were used in assays employing a limited set of primary probes targeting mouse eef2 mRNA.

Primary probes. 24 probes were designed to target mouse eef2 mRNA. The RNA binding region length was 35 nucleotides for the non-split configuration and 21+21 nucleotides for the split configuration. Each probe contains one RNA binding site and one to three secondary amplifier-anchor binding sites.

Anchors. Anchor oligonucleotides consisted of 15 nucleotides DNA modified with a 5′ alkyne and a 3′ BBA group. Oligos with 5′-alkyne and 3′-amine were purchased from IDT. The 3′-BBA modification was introduced using 4-benzoylbenzoic acid N-succinimidyl ester, followed by purification via RP-HPLC. Distinct anchor sequences were used for secondary and tertiary amplification stages.

Amplifiers. Each amplifier contained (i) a sequence complementary to the amplification site of the preceding probe, (ii) two repeats of its orthogonal amplification site, and (iii) the two corresponding anchor sites. N⁶-(6-azido)hexyl-dATP was incorporated at the 3′ end through TdT-mediated extension.

Hybridization and Split-Assay CuAAC Reaction. After charge quenching, flow in the hybridization buffer containing 5 nM/oligo primary probe, 0.1 mg/ml yeast tRNA, and 2 M polyTTG (200 nucleotides) in 10% 40 kDa Dextran Sulfate and 25% formamide. The sample was incubated at 37 °C for 18 hours. For split-primary-probe assay, a primary CuAAC step was performed. Fresh click buffer (2×SSC, 75 μM CuSO₄, 375 μM BTTAA, 0.25% Triton X-100, 5% DMSO, and 3 mM (+)-sodium L-ascorbate) was prepared and applied for 1 h at 37 °C. 60% formamide wash was performed afterwards at 37 C to remove nonspecific sticking.

### Amplification Procedure

Amplification was conducted over 12 sequential rounds. Generally, each round consisted of:
Hybridization of secondary or tertiary amplifiers (50 nM) and anchors (75 nM) for 1 h at 37 °C;
Apply 10% formamide wash buffer to remove nonspecific binding.
Incubation with CuAAC click buffer for 60 min at 37 C; Apply 30% formamide wash buffer to remove nonspecific binding.
UV irradiation at 365 nm (~130 mW) for 10 min at RT to induce photocrosslinking.
Removal of unligated/photocrosslinked oligonucleotides by applying 60% formamide wash for 10 min.

### Example 2

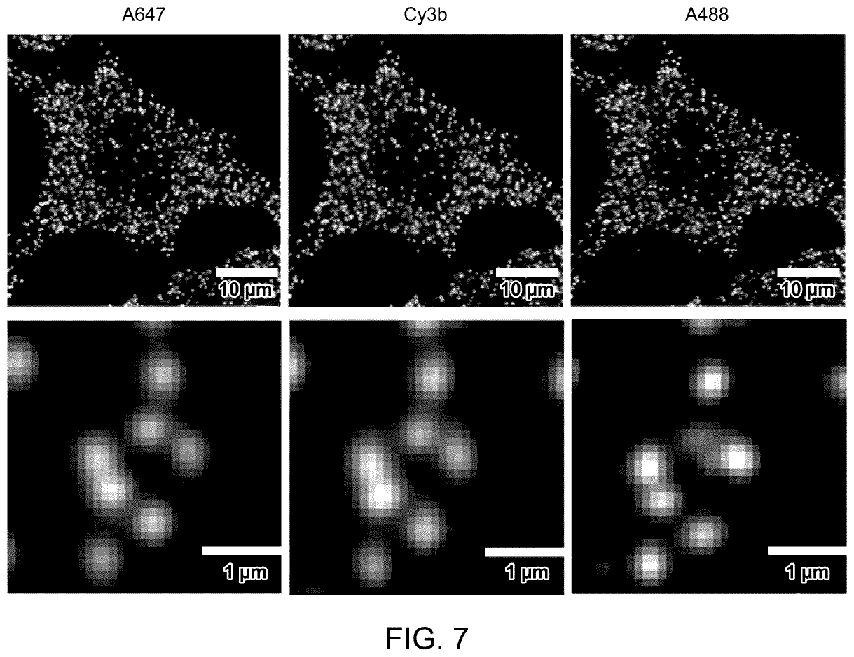

In one example, 24 probes were designed to specifically hybridize to the eef2 mRNA sequence. (Table 1, Left and Right). Each probe comprised a 25 nucleotide target-binding region and one or more amplifier-anchor binding sites. After amplification (using the sequences in Tables 2 and 3), each probe was recognized by three fluorescent readout probes incorporating fluorophores A647, Cy3b, and A488. During image acquisition, the A647, Cy3b, and A488 channels were exposed for 500 ms, 250 ms, and 500 ms, respectively. The fluorescent signals observed across the three channels demonstrated strong signal intensity (~30 fold higher than non-amplified sample) and a colocalization ratio between two channels of 90%, as shown in FIG. 7.

TABLE 1

| Probe | |
|---|---|
| Left | Sequence |
| 1 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACATGGGCGATG |
| | ACTGACATGTTCCGACCCTATAGTGAGTCGTATTACCGGCC |
| 2 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATATCATCCTTGCGA |
| | GTGTCAGTGAAGCACCCTATAGTGAGTCGTATTACCGGCC |
| 3 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGAGGAAGCCCG |
| | AGCCATCCTTGCTCACCCTATAGTGAGTCGTATTACCGGCC |
| 4 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGCTGTCACCTCT |
| | GAAGAGAAGTCCAACCCTATAGTGAGTCGTATTACCGGCC |
| 5 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACACAGTTTCTGT |
| | CTGCACACACACGACCCTATAGTGAGTCGTATTACCGGCC |
| 6 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAAGGTCTGGTAG |
| | AGCTCCTCGGGCTCACCCTATAGTGAGTCGTATTACCGGCC |
| 7 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGCCCTCGCCGT |
| | AGGTAGAGATGATGACCCTATAGTGAGTCGTATTACCGGCC |
| 8 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATATCTCCGCAAACT |
| | GCTTCAGGGTGAAACCCTATAGTGAGTCGTATTACCGGCC |
| 9 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACTCGGCTGCGC |
| | TCAGCTGGCCCTCAACCCTATAGTGAGTCGTATTACCGGCC |
| 10 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACTGAAGTTCAT |
| | GATGGCGTCGAACAACCCTATAGTGAGTCGTATTACCGGCC |
| 11 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACGCTGTCCAGC |
| | TTGATGTCCAGCTTACCCTATAGTGAGTCGTATTACCGGCC |
| 12 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAACGGTACTTCT |
| | GTGCAGTGACGGGGACCCTATAGTGAGTCGTATTACCGGCC |
| 13 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAAAGCGGCCTTT |
| | GTCAGAGGTTGGCAACCCTATAGTGAGTCGTATTACCGGCC |
| 14 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACGTAGCGGCCC |
| | ATCATCAGAATGGTACCCTATAGTGAGTCGTATTACCGGCC |
| 15 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACACTCCGACCA |
| | GCCCCACAATGTTTACCCTATAGTGAGTCGTATTACCGGCC |
| 16 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATATTGTGAGCGTG |
| | CTCAAAGGTAGTGAACCCTATAGTGAGTCGTATTACCGGCC |
| 17 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATATGTTCTCCAGAC |
| | TCCTCAATGATGCACCCTATAGTGAGTCGTATTACCGGCC |
| 18 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACCAGGTCCTTAA |
| | GGCAGATCTCCAGACCCTATAGTGAGTCGTATTACCGGCC |
| 19 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGTACAGCCGGT |
| | TGTGCTTATTGGGGACCCTATAGTGAGTCGTATTACCGGCC |
| 20 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGCCATCAGGGC |
| | CAAAGCACCAGATCACCCTATAGTGAGTCGTATTACCGGCC |
| 21 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATATCATTCAGGTAC |
| | TGCACACCCTTGGACCCTATAGTGAGTCGTATTACCGGCC |
| 22 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATACGCATGTTTTCC |
| | TCACAGAGAGCGCACCCTATAGTGAGTCGTATTACCGGCC |
| 23 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAATGCCACCCACC |
| | ACTTGCTCAGGACACCCTATAGTGAGTCGTATTACCGGCC |
| 24 | GCCCCATCATGTGCCTTTCCTTTGACGAAGCCTGACAATGACGCGACATAGATTGACGAAGCC |
| | TGACGTACAACCTCGGTTAATTGACGAAGCCTGACAGCTCGGCCTGTGATAGCCAGTGGTCA |
| | AACACGCACTGGGGACCCTATAGTGAGTCGTATTACCGGCC |
| Probe | |
| Right | Sequence |
| 1 | GCCCCATCATGTGCCTTTCCTGGTCAGCGTGGACTTGCCGTGGTCCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 2 | GCCCCATCATGTGCCTTTCCTATTTGATTGTGATGCAGCGCTCCTGATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 3 | GCCCCATCATGTGCCTTTCCTTGGCCTGGAGAGTCGATGAGGTTGAATTGACGAAGCCTGAC |
| | CCGCAAGTAATCTGTATTGACGAAGCCTGACCGTCGATAGGACTCAATTGACGAAGCCTGAC |
| | CTGGTGATAACGCTAACCCTATAGTGAGTCGTATTACCGGCC |
| 4 | GCCCCATCATGTGCCTTTCCTGAGCTCCATCGGTGACACGCAAGGCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 5 | GCCCCATCATGTGCCTTTCCTATGCGTTCAGCGATGGCCTGGCGCAATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 6 | GCCCCATCATGTGCCTTTCCTGTTGACGTTCTCCACGATGCGCTGGATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 7 | GCCCCATCATGTGCCTTTCCTATGATATTGCCCATGGGCCCACTCTATTGACGAAGCCTGACCC |
| | GCAAGTAATCTGTATTGACGAAGCCTGACCGTCGATAGGACTCAATTGACGAAGCCTGACCT |
| | GGTGATAACGCTAACCCTATAGTGAGTCGTATTACCGGCC |
| 8 | GCCCCATCATGTGCCTTTCCTCTTGGCTGCAAACTTGGCCACATACATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 9 | GCCCCATCATGTGCCTTTCCTATCATGTCCTCTACTTTCTTGGCACATTGACGAAGCCTGACCC |
| | GCAAGTAATCTGTATTGACGAAGCCTGACCGTCGATAGGACTCAATTGACGAAGCCTGACCT |
| | GGTGATAACGCTAACCCTATAGTGAGTCGTATTACCGGCC |
| 10 | GCCCCATCATGTGCCTTTCCTCGATCAGCTTGGCTGTCTCCTCCTTATTGACGAAGCCTGACCC |
| | GCAAGTAATCTGTATTGACGAAGCCTGACCGTCGATAGGACTCAATTGACGAAGCCTGACCT |
| | GGTGATAACGCTAACCCTATAGTGAGTCGTATTACCGGCC |
| 11 | GCCCCATCATGTGCCTTTCCTTGGCTTGCCCTCCTTGTCTTTGTCCATTGACGAAGCCTGACCC |
| | GCAAGTAATCTGTATTGACGAAGCCTGACCGTCGATAGGACTCAATTGACGAAGCCTGACCT |
| | GGTGATAACGCTAACCCTATAGTGAGTCGTATTACCGGCC |
| 12 | GCCCCATCATGTGCCTTTCCTGGAGGCCCCTCGTATAGCAGCTCACATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 13 | GCCCCATCATGTGCCTTTCCTCAGAGAACACTCTACCAAAGGCATAATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 14 | GCCCCATCATGTGCCTTTCCTACATGGCACGTCCTCAATCGGCTCCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 15 | GCCCCATCATGTGCCTTTCCTGTCCCCGTCTTCACAAGGAACTGGTATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 16 | GCCCCATCATGTGCCTTTCCTTGACGCTGAACTTCATCACGCGCATATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 17 | GCCCCATCATGTGCCTTTCCTGCAGCTCACCGGCGCCAGCAATAATATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 18 | GCCCCATCATGTGCCTTTCCTGATGGGGATGCAGGCATGGTCCTCCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 19 | GCCCCATCATGTGCCTTTCCTCCATCAGGGAAGGGCCTGGCCTTCAATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 20 | GCCCCATCATGTGCCTTTCCTATGTCGGTGAGAATGTTGGGGCCAGATTGACGAAGCCTGAC |
| | CCGCAAGTAATCTGTATTGACGAAGCCTGACCGTCGATAGGACTCAATTGACGAAGCCTGAC |
| | CTGGTGATAACGCTAACCCTATAGTGAGTCGTATTACCGGCC |
| 21 | GCCCCATCATGTGCCTTTCCTAGCCAGCCACCACACTGTCCTTGATATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 22 | GCCCCATCATGTGCCTTTCCTCATCATGAACATCAAACCGCACACCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 23 | GCCCCATCATGTGCCTTTCCTCACGCTTCCTGTTCAGGACACCGTAATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |
| 24 | GCCCCATCATGTGCCTTTCCTGTCAAAAGGATCCCCAGGCAGGATCATTGACGAAGCCTGACC |
| | CGCAAGTAATCTGTATTGACGAAGCCTGACCGTCGATAGGACTCAATTGACGAAGCCTGACC |
| | TGGTGATAACGCTAACCCTATAGTGAGTCGTATTACCGGCC |

TABLE 2
Secondary amplifiers

| Amplifier | Sequence |
|---|---|
| 1 | GCCCCATCATGTGCCTTTCCTTACCCGTTGAGTGGCAGGGTATCTCGCGTCTACCCGTTGA |
| | GTGGCAGGGTATCTCGCGTCCTATGTCGCGTCATTACCCTATAGTGAGTCGTATTACCGGC |
| | C |
| 2 | GCCCCATCATGTGCCTTTCCTTACCCGTTGAGTGGCAGATTAGCGCCGTATTACCCGTTGA |
| | GTGGCAGATTAGCGCCGTATATCACAGGCCGAGCTACCCTATAGTGAGTCGTATTACCGG |
| | CC |
| 3 | GCCCCATCATGTGCCTTTCCTTACCCGTTGAGTGGCAAGAGTACCGTCGATTACCCGTTGA |
| | GTGGCAAGAGTACCGTCGATTAGCGTTATCACCAGACCCTATAGTGAGTCGTATTACCGG |
| | CC |
| 4 | GCCCCATCATGTGCCTTTCCTTACCCGTTGAGTGGCCCTAGACTGATCGCGTACCCGTTGA |
| | GTGGCCCTAGACTGATCGCGACAGATTACTTGCGGACCCTATAGTGAGTCGTATTACCGG |
| | CC |
| 5 | GCCCCATCATGTGCCTTTCCTTACCCGTTGAGTGGCTGGCCTAATCGCGAATACCCGTTGA |
| | GTGGCTGGCCTAATCGCGAATAACCGAGGTTGTACACCCTATAGTGAGTCGTATTACCGG |
| | CC |
| 6 | GCCCCATCATGTGCCTTTCCTTACCCGTTGAGTGGCCAATCTTCGCGAGTATACCCGTTGA |
| | GTGGCCAATCTTCGCGAGTATGAGTCCTATCGACGACCCTATAGTGAGTCGTATTACCGGC |
| | C |

TABLE 3
Tertiary amplifiers

| Amplifier | Sequence |
|---|---|
| 1 | GCCCCATCATGTGCCTTTCCtTACCCGTTGAGTGGCAATGACGCGACATAGTACCCGTTGAG |
| | TGGCAATGACGCGACATAGGACGCGAGATACCCTaCCCTATAGTGAGTCGTATTACCGGCC |
| 2 | GCCCCATCATGTGCCTTTCCtTACCCGTTGAGTGGCAGCTCGGCCTGTGATTACCCGTTGAG |
| | TGGCAGCTCGGCCTGTGATATACGGCGCTAATCTaCCCTATAGTGAGTCGTATTACCGGCC |
| 3 | GCCCCATCATGTGCCTTTCCtTACCCGTTGAGTGGCCTGGTGATAACGCTATACCCGTTGAG |
| | TGGCCTGGTGATAACGCTAATCGACGGTACTCTTaCCCTATAGTGAGTCGTATTACCGGCC |
| 4 | GCCCCATCATGTGCCTTTCCtTACCCGTTGAGTGGCCCGCAAGTAATCTGTTACCCGTTGAG |
| | TGGCCCGCAAGTAATCTGTCGCGATCAGTCTAGGaCCCTATAGTGAGTCGTATTACCGGCC |
| 5 | GCCCCATCATGTGCCTTTCCtTACCCGTTGAGTGGCGTACAACCTCGGTTATACCCGTTGAG |
| | TGGCGTACAACCTCGGTTATTCGCGATTAGGCCAaCCCTATAGTGAGTCGTATTACCGGCC |
| 6 | GCCCCATCATGTGCCTTTCCtTACCCGTTGAGTGGCCGTCGATAGGACTCATACCCGTTGAG |
| | TGGCCGTCGATAGGACTCATACTCGCGAAGATTGaCCCTATAGTGAGTCGTATTACCGGCC |

### Example 3

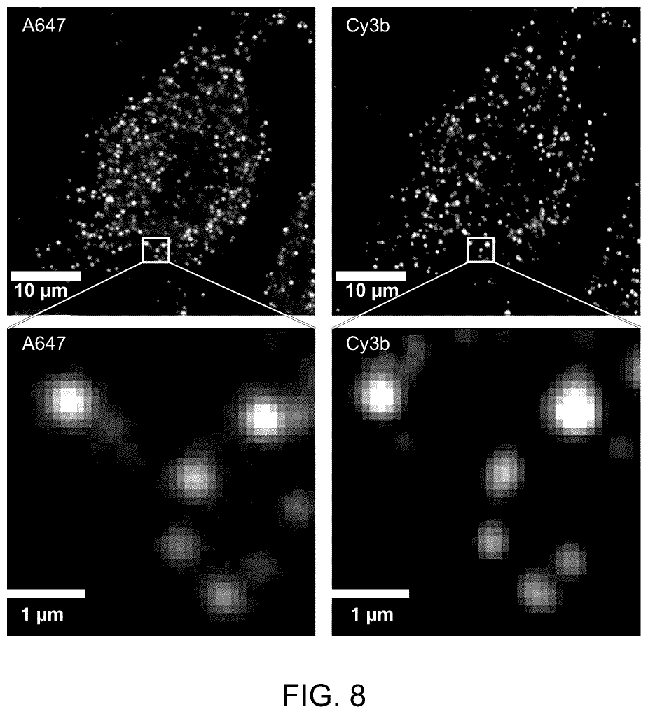

In one example, one probe (Table 4) was designed to specifically hybridize to the eef2 mRNA sequence, wherein each probe comprises a binding sequence of 35 nucleotides in length, and 3 repeats of the same amplifier-anchor binding sites. The schematics of amplification are similar to those shown in FIG. 21. Each probe can be recognized by two fluorescent readout probes comprising fluorophores A647 and Cy3b after amplification. The amplification (using the sequences from tables 5 and 6) was performed as previously described. During image acquisition, both channels were exposed for 2000 ms respectively. As a result, the fluorescent signals detected in the three channels exhibited high signal to noise ratio and high colocalization ratio of 71%, as shown in FIG. 8.

TABLE 4

| | |
|---|---|
| 35 | GCCCCATCATGTGCCTTTCCTTGACGAAGCCTGACGGTCTCGGATATGCAAATGATGGC |
| nucleotide | GCGGATCTGGTCTACCGTGAAGTTCACATTGACGAAGCCTGACTGCGTACGACAATGT |
| probe | ACCCTATAGTGAGTCGTATTACCGGCC |

TABLE 5
Secondary amplifiers

| Amplifier | Sequence |
|---|---|
| 1 | GCCCCATCATGTGCCTTTCCTACCCGTTGAGTGGCAAATAGGTTCCCGTTCGATACCCGT |
| | TGAGTGGCAAATAGGTTCCCGTTCGATGGCGAGAATTCATGACCCTATAGTGAGTCGTA |
| | TTACCGGCC |
| 2 | GCCCCATCATGTGCCTTTCCTACCCGTTGAGTGGCAACCTTTAGACGCCGTAATACCCGT |
| | TGAGTGGCAACCTTTAGACGCCGTAACGCGAAGATGCAATTACCCTATAGTGAGTCGTA |
| | TTACCGGCC |
| 3 | GCCCCATCATGTGCCTTTCCTACCCGTTGAGTGGCAAAGATTAGCGCCGTATATACCCGT |
| | TGAGTGGCAAAGATTAGCGCCGTATAATCACAGGCCGAGCTACCCTATAGTGAGTCGTA |
| | TTACCGGCC |
| 4 | GCCCCATCATGTGCCTTTCCTACCCGTTGAGTGGCAAACTAGTCGACCGTCCATACCCGT |
| | TGAGTGGCAAACTAGTCGACCGTCCAATACCGGCCTTGAGTACCCTATAGTGAGTCGTA |
| | TTACCGGCC |
| 5 | GCCCCATCATGTGCCTTTCCTACCCGTTGAGTGGCAAATACCTTCCGCGTATATACCCGT |
| | TGAGTGGCAAATACCTTCCGCGTATAAACGCTGTACGAGTGACCCTATAGTGAGTCGTA |
| | TTACCGGCC |
| 6 | GCCCCATCATGTGCCTTTCCTACCCGTTGAGTGGCAACGGCTATACTCGTACATACCCGT |
| | TGAGTGGCAACGGCTATACTCGTACAACAGTTCGCGTATCAACCCTATAGTGAGTCGTA |
| | TTACCGGCC |
| 7 | GCCCCATCATGTGCCTTTCCTACCCGTTGAGTGGCAAGGTTTAGACGTCGACATACCCGT |
| | TGAGTGGCAAGGTTTAGACGTCGACATGCATATCCGAGACCACCCTATAGTGAGTCGTA |
| | TTACCGGCC |
| 8 | GCCCCATCATGTGCCTTTCCTACCCGTTGAGTGGCAACGAGATTAGCGCGGTATACCCG |
| | TTGAGTGGCAACGAGATTAGCGCGGTAACATTGTCGTACGCAACCCTATAGTGAGTCGT |
| | ATTACCGGCC |

TABLE 6
Tertiary amplifiers

| Amplifier | Sequence |
|---|---|
| 1 | GCCCCATCATGTGCCTTTCCTTGACGAAGCCTGACAACATGAATTCTCGCCAATTGACGA |
| | AGCCTGACAACATGAATTCTCGCCAACGAACGGGAACCTATACCCTATAGTGAGTCGTA |
| | TTACCGGCC |
| 2 | GCCCCATCATGTGCCTTTCCTTGACGAAGCCTGACAAAATTGCATCTTCGCGATTGACGA |
| | AGCCTGACAAAATTGCATCTTCGCGATACGGCGTCTAAAGGACCCTATAGTGAGTCGTA |
| | TTACCGGCC |
| 3 | GCCCCATCATGTGCCTTTCCTTGACGAAGCCTGACAAAGCTCGGCCTGTGATATTGACG |
| | AAGCCTGACAAAGCTCGGCCTGTGATAATACGGCGCTAATCTACCCTATAGTGAGTCGT |
| | ATTACCGGCC |
| 4 | GCCCCATCATGTGCCTTTCCTTGACGAAGCCTGACAAACTCAAGGCCGGTATATTGACG |
| | AAGCCTGACAAACTCAAGGCCGGTATAGGACGGTCGACTAGTACCCTATAGTGAGTCG |
| | TATTACCGGCC |
| 5 | GCCCCATCATGTGCCTTTCCTTGACGAAGCCTGACAACACTCGTACAGCGTTATTGACGA |
| | AGCCTGACAACACTCGTACAGCGTTAATACGCGGAAGGTATACCCTATAGTGAGTCGTA |
| | TTACCGGCC |
| 6 | GCCCCATCATGTGCCTTTCCTTGACGAAGCCTGACAATGATACGCGAACTGTATTGACG |
| | AAGCCTGACAATGATACGCGAACTGTAGTACGAGTATAGCCGACCCTATAGTGAGTCGT |
| | ATTACCGGCC |
| 7 | GCCCCATCATGTGCCTTTCCTTGACGAAGCCTGACAAGGTCTCGGATATGCAATTGACG |
| | AAGCCTGACAAGGTCTCGGATATGCAAGTCGACGTCTAAACCACCCTATAGTGAGTCGT |
| | ATTACCGGCC |
| 8 | GCCCCATCATGTGCCTTTCCTTGACGAAGCCTGACAATGCGTACGACAATGTATTGACG |
| | AAGCCTGACAATGCGTACGACAATGTAACCGCGCTAATCTCGACCCTATAGTGAGTCGT |
| | ATTACCGGCC |

### Example 4

In one example, a pair of probes (Table 7) were designed to hybridize to two adjacent sites on the eef2 mRNA. Each probe consisted of a 21 nucleotides hybridization sequence and three repeated amplifier-anchor binding sites. The schematics of amplification are similar to those shown in FIG. 21. To enable subsequent chemical ligation, the left probe was modified with a 3′ azide group and the right probe with a 5′ alkyne group. Upon hybridization, the two probes aligned contiguously on the mRNA, positioning their reactive groups in proximity.

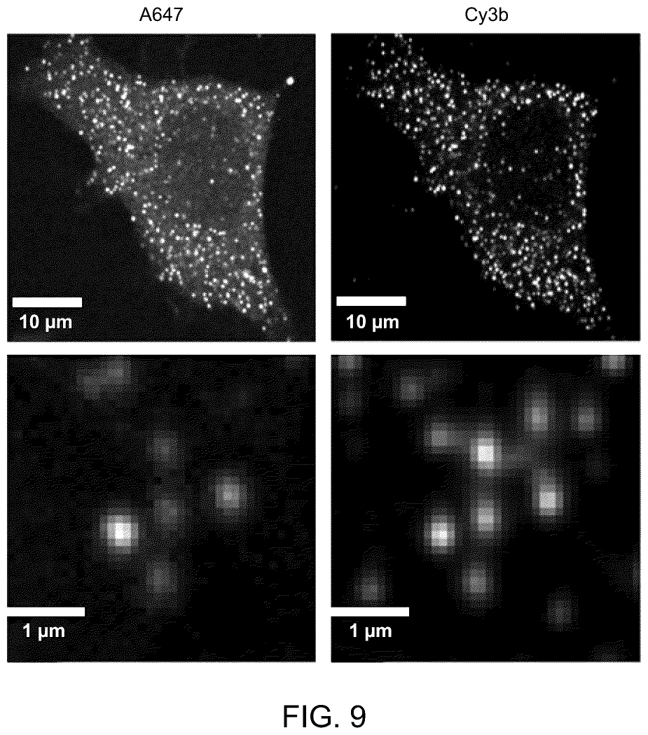

Following amplification (using the sequences from Tables 2 and 3), each transcript was detected by two fluorescent readout probes labeled with A647 and Cy3b. During confocal imaging, both fluorescence channels were acquired with a 2000 ms exposure time. The resulting images exhibited strong signal intensities and a colocalization ratio of 87%, as shown in FIG. 9.

TABLE 7

| | |
|---|---|
| left | GCCCCATCATGTGCCTTTCCTTGACGAAGCCTGACAGCTCGGCCTGTGATATTGACG |
| | AAGCCTGACCCGCAAGTAATCTGTAGGTCAGCGTGGACTTGCCGTGACCCTATAGT |
| | GAGTCGTATTACCGGCC |
| right | GCCCCATCATGTGCCTTTCCGTCCACATGGGCGATGACTGAATTGACGAAGCCTGAC |
| | GTACAACCTCGGTTAATTGACGAAGCCTGACCGTCGATAGGACTCAACCCTATAGT |
| | GAGTCGTATTACCGGCC |

### Example 5

Tissue sections were prepared at a thickness of 10 m and fixed in 4% PFA in 1×PBS at room temperature for 10 min. The sample was permeabilized by 8% Triton X-100 for 1 hour at room temperature. Subsequent sample-preparation steps followed the protocol used for fixed-cell samples, except that charge quenching was performed four times for 1 h each. The probe design was similar to that used in example 2, except that each probe contained a 35-nucleotide binding region instead of 25 nucleotides (Table 8).

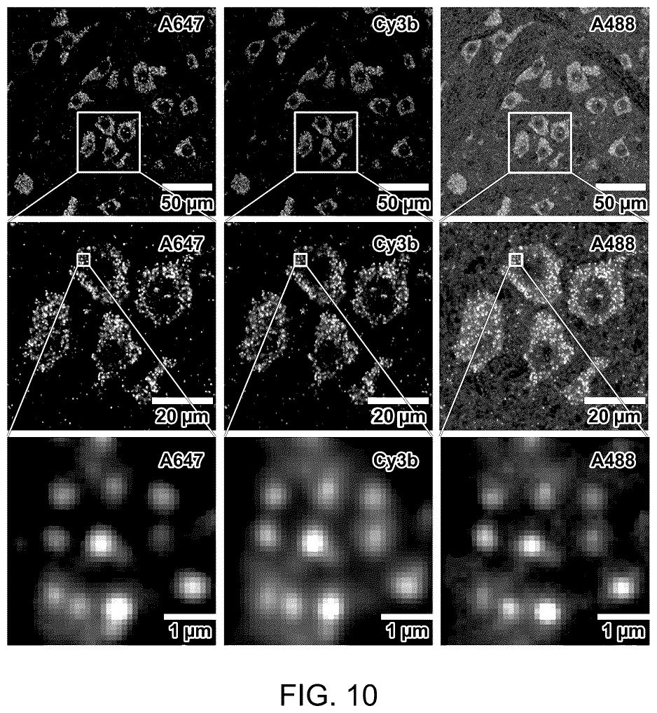

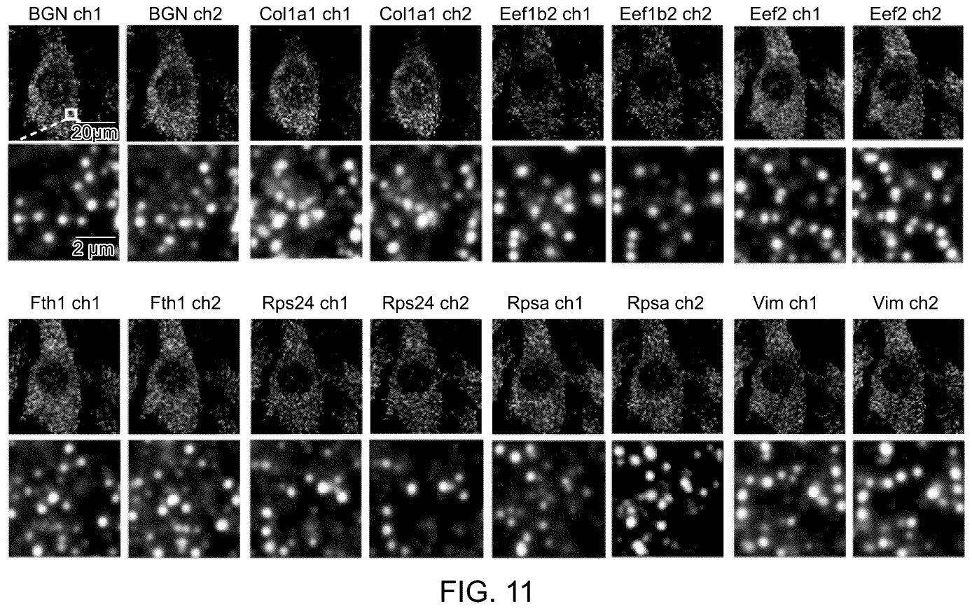

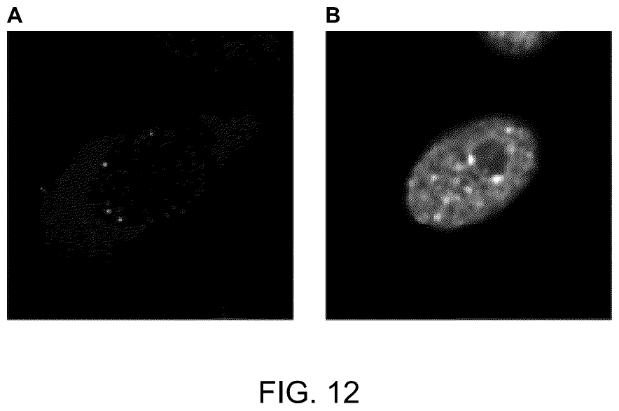

Amplification (using the sequences from Tables 5 and 6) was performed according to the standard procedure, with two modifications: (i) the hybridization time for the amplifier and anchor probes was extended to 2 hours, and (ii) the CuAAC click buffer was used at a higher concentration (2×SSC, 150 μM CuSO₄, 750 μM BTTAA, 0.25% o Triton X-100, 500 DMSO, and 6 mM (+)-sodium L-ascorbate). The schematics of amplification are similar to those shown in FIG. 21. During image acquisition, all three fluorescence channels were exposed for 2000 ins. Clear fluorescent signals and strong colocalization (810%) were observed, demonstrating the excellent compatibility of this method with tissue samples. FIGS. 10, 11, and 12.

TABLE 8

| Probe | Sequence |
|---|---|
| 1 | GCCCCATCATGTGCCTTTCcTTGACGAAGCCTGACGGTCTCGGATATGCAaA |
| | TGATGGCGCGGATCTGGTCTACCGTGAAGTTCACaTTGACGAAGCCTGACT |
| | GCGTACGACAATGTaCCCTATAGTGAGTCGTATTACCGGCC |
| 2 | GCCCCATCATGTGCCTTTCcTTGACGAAGCCTGACGGTCTCGGATATGCAaA |
| | GTCTGTCAGCGTGGACTTGCCATGGTCCACGTGGaTTGACGAAGCCTGACT |
| | GCGTACGACAATGTaCCCTATAGTGAGTCGTATTACCGGCC |
| 3 | GCCCCATCATGTGCCTTTCcTTGACGAAGCCTGACGGTCTCGGATATGCAaC |
| | ATTCTCCGAGAGCTCGTAGAAGAGGGAGATGGCAaTTGACGAAGCCTGAC |
| | TGCGTACGACAATGTaCCCTATAGTGAGTCGTATTACCGGCC |
| 4 | GCCCCATCATGTGCCTTTCcTTGACGAAGCCTGACGGTCTCGGATATGCAaC |
| | CGGCACCGTCCTTGCTCTGCTTGATGAAGTTCAAaTTGACGAAGCCTGACT |
| | GCGTACGACAATGTaCCCTATAGTGAGTCGTATTACCGGCC |
| 5 | GCCCCATCATGTGCCTTTCcTTGACGAAGCCTGACGGTCTCGGATATGCAaT |
| | CATCATCAGCACAGGCTTGATGCGCTCGGCAATGaTTGACGAAGCCTGACT |
| | GCGTACGACAATGTaCCCTATAGTGAGTCGTATTACCGGCC |
| 6 | GCCCCATCATGTGCCTTTCcTTGACGAAGCCTGACGGTCTCGGATATGCAaC |
| | ACGATGCGCTGGAAAGTCTGGTAGAGCTCCTCGGaTTGACGAAGCCTGACT |
| | GCGTACGACAATGTaCCCTATAGTGAGTCGTATTACCGGCC |
| 7 | GCCCCATCATGTGCCTTTCcTTGACGAAGCCTGACGGTCTCGGATATGCAaC |
| | CTCGCCGTAGGTGGAGATGATGACGTTCACGTTCaTTGACGAAGCCTGACT |
| | GCGTACGACAATGTaCCCTATAGTGAGTCGTATTACCGGCC |
| 8 | GCCCCATCATGTGCCTTTCcTTGACGAAGCCTGACGGTCTCGGATATGCAaG |
| | ACCCAAAGCCCACGGTACCGAGGACAGGATCGATaTTGACGAAGCCTGAC |
| | TGCGTACGACAATGTaCCCTATAGTGAGTCGTATTACCGGCC |
| 9 | GCCCCATCATGTGCCTTTCcTTGACGAAGCCTGACGGTCTCGGATATGCAaA |
| | CTTGCTGAACTTGCCGTTGGCTGGGTCAAAGTACaTTGACGAAGCCTGACT |
| | GCGTACGACAATGTaCCCTATAGTGAGTCGTATTACCGGCC |
| 10 | GCCCCATCATGTGCCTTTCcTTGACGAAGCCTGACGGTCTCGGATATGCAaG |
| | CAGGGGTTTGCCTTCTTTGTCCTTGTCCTCGCTGaTTGACGAAGCCTGACTG |
| | CGTACGACAATGTaCCCTATAGTGAGTCGTATTACCGGCC |
| 11 | GCCCCATCATGTGCCTTTCcTTGACGAAGCCTGACGGTCTCGGATATGCAaA |
| | GGGGAGGGCAGGTGGATGGTGATCATCTGCAACAaTTGACGAAGCCTGAC |
| | TGCGTACGACAATGTaCCCTATAGTGAGTCGTATTACCGGCC |
| 12 | GCCCCATCATGTGCCTTTCcTTGACGAAGCCTGACGGTCTCGGATATGCAaA |
| | GCCCCGAGAAGACTCGTCCAAAGGCGTAGAACCGaTTGACGAAGCCTGAC |
| | TGCGTACGACAATGTaCCCTATAGTGAGTCGTATTACCGGCC |
| 13 | GCCCCATCATGTGCCTTTCcTTGACGAAGCCTGACGGTCTCGGATATGCAaT |
| | CAGGTAGAGGTCCTCCTTCTTCCCAGGGGTATAGaTTGACGAAGCCTGACT |
| | GCGTACGACAATGTaCCCTATAGTGAGTCGTATTACCGGCC |
| 14 | GCCCCATCATGTGCCTTTCcTTGACGAAGCCTGACGGTCTCGGATATGCAaT |
| | GGGCTCCACGTAGCGGCCCATCATCAAGATTGTTaTTGACGAAGCCTGACT |
| | GCGTACGACAATGTaCCCTATAGTGAGTCGTATTACCGGCC |
| 15 | GCCCCATCATGTGCCTTTCcTTGACGAAGCCTGACGGTCTCGGATATGCAaA |
| | CGAGGCCCACAATGTTCCCACAAGGCACATCCTCaTTGACGAAGCCTGACT |
| | GCGTACGACAATGTaCCCTATAGTGAGTCGTATTACCGGCC |
| 16 | GCCCCATCATGTGCCTTTCcTTGACGAAGCCTGACGGTCTCGGATATGCAaT |
| | CATCACCCGCATGTTGTGCGCGTGCTCGAAGGTGaTTGACGAAGCCTGACT |
| | GCGTACGACAATGTaCCCTATAGTGAGTCGTATTACCGGCC |
| 17 | GCCCCATCATGTGCCTTTCcTTGACGAAGCCTGACGGTCTCGGATATGCAaT |
| | CCACGGCCACTCTGACAACAGGGCTGACGCTGAAaTTGACGAAGCCTGACT |
| | GCGTACGACAATGTaCCCTATAGTGAGTCGTATTACCGGCC |
| 18 | GCCCCATCATGTGCCTTTCcTTGACGAAGCCTGACGGTCTCGGATATGCAaG |
| | CGATGATATGCTCTCCCGACTCCTCGATGATGCAaTTGACGAAGCCTGACT |
| | GCGTACGACAATGTaCCCTATAGTGAGTCGTATTACCGGCC |
| 19 | GCCCCATCATGTGCCTTTCcTTGACGAAGCCTGACGGTCTCGGATATGCAaA |
| | CCGTCTCGCGGTACGAGACGACCGGGTCAGATTTaTTGACGAAGCCTGACT |
| | GCGTACGACAATGTaCCCTATAGTGAGTCGTATTACCGGCC |
| 20 | GCCCCATCATGTGCCTTTCcTTGACGAAGCCTGACGGTCTCGGATATGCAaG |
| | GACTTGGAGAGGCAGAGCACGTTCGACTCTTCACaTTGACGAAGCCTGACT |
| | GCGTACGACAATGTaCCCTATAGTGAGTCGTATTACCGGCC |
| 21 | GCCCCATCATGTGCCTTTCcTTGACGAAGCCTGACGGTCTCGGATATGCAaG |
| | CCGCGCCTTCATGTACAGCCGGTTGTGCTTGTTGaTTGACGAAGCCTGACT |
| | GCGTACGACAATGTaCCCTATAGTGAGTCGTATTACCGGCC |
| 22 | GCCCCATCATGTGCCTTTCcTTGACGAAGCCTGACGGTCTCGGATATGCAaC |
| | GGCCACCACACTGTCCTTGATCTCGTTGAGGTACaTTGACGAAGCCTGACT |
| | GCGTACGACAATGTaCCCTATAGTGAGTCGTATTACCGGCC |
| 23 | GCCCCATCATGTGCCTTTCcTTGACGAAGCCTGACGGTCTCGGATATGCAaT |
| | GACGGGCAGATAGGCCTTGACCACAAACATGGGGaTTGACGAAGCCTGAC |
| | TGCGTACGACAATGTaCCCTATAGTGAGTCGTATTACCGGCC |
| 24 | GCCCCATCATGTGCCTTTCcTTGACGAAGCCTGACGGTCTCGGATATGCAaA |
| | ATTTGTCCAGGAAGTTGTCCAGGGCAGGGATGCCaTTGACGAAGCCTGACT |
| | GCGTACGACAATGTaCCCTATAGTGAGTCGTATTACCGGCC |

### Example 6

### Read-Anchor Strategy can be Used for Detection of Interactions

We used NIH3T3 cells as samples to test the design for both the read-anchor interaction detector (coincidence detector). The NIH3T3 cells were first fixed with 4% PFA for ten minutes and permeabilized with 70% ethanol overnight. They were then treated with N-(Propionyloxy)succinimide to reduce nonspecific binding.

### Primary Probe Pairs and Bridge:

The target of interest used in the development of this method as a proof of concept is 18s rRNA. Primary A, Primary B Grab, and Primary B Loop probes (Table 9) were designed to have an rRNA binding region, an read(amplifier)/anchor probe binding region, and a grab/loop region. Optionally, the primary probes may contain a primary readout region. The rRNA binding region allows the probes to bind to designated rRNA sequences specifically. The primary readout region allows us to visually validate the presence of these primary probes after the click reaction and UV photo-crosslinking. The amplifier/anchor probe binding region allows the amplifier and anchor with functional modifications to bind to the corresponding primary probes. The grab and the loop regions are critical for the click reaction to occur, as they enforce a physical constraint to bring the primary probes close to each other. For the grab configuration, Primary A and Primary B Grab work as a pair, and their grab regions are designed to be complementary to each other; the loop configuration, on the other hand, requires Primary A, Primary B Loop, and the Loop Bridge to work together, and the Loop Bridge is designed to bind partially to Primary A and partially to Primary B Loop.

TABLE 9

| | |
|---|---|
| Primary_A | ATCCTGAACATGCCTTGATCGATCGTAATCAAAGGCCGCAGATTTAATGAGCCA |
| | TTCGCAGTTTCACTGT |
| Primary_B_grab | GAGGAGCGAGCGACCAAAGGAACCATAACTGACGGTTGTAACGGTACCCGTT |
| | GAGTGGCTGTTCAGGAT |
| Primary_B_loop | AGAGGAGCGAGCGACCAAAGGAACCATAACTGACGGTTGTAACGGTACCCGT |
| | TGAGTGGCTGGCGAGAAT |
| loop_bridge | TGTTCAGGATTTTTTATTCTCGCCA |

### Read (Amplifier), Anchor and Readout Probes:

The amplifier consists of two parts: a primary binding region and an amplifier readout region. The primary binding region is complementary to the amplifier probe binding region on the primary probe, and the amplifier readout region is used for the final readout. The amplifier was functionalized by adding an azide group to the 3′ end using Terminal Deoxynucleotidyl Transferase (TdT). The anchor only has a primary binding region which is complementary to the anchor probe binding region on the primary probes. An anchor oligo from IDT with an amine on the 5′ end was obtained. The anchor was functionalized with a photo-crosslinker via an NHS-amine reaction, and added an alkyne group on the 3′ end using TdT. The readout probe was made by coupling the fluorophores and the oligo via an NHS-amine reaction (Table 10).

TABLE 10

| | |
|---|---|
| Ter anchor | /5Hexynyl/GTCAGGCTTCGTC/iAmMC6T/ |
| | A/iAmMC6T/A/3AmMO/ |
| Sec anchor | /5Hexynyl/GCCACTCAACGGG/iAmMC6T/ |
| | T/iAmMC6T/A/3AmMO/ |

### Experiment Procedures:

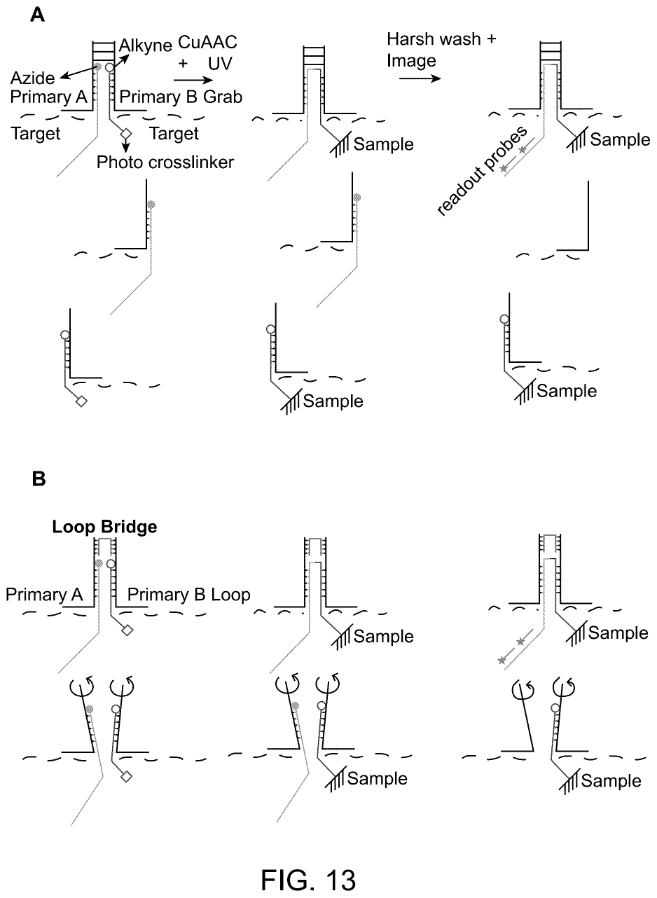

Primary probe pairs were first hybridized in the sample in a 30% formamide solution overnight (exact conditions depended on the length of the rRNA binding region). Next, the sample was washed with a 35% formamide solution to remove off-target probes. Note the primary probes do not need to be crosslinked or clicked. Then, if using loop configurations, the loop bridge was hybridized to the primary probe pairs in 10% formamide. The amplifier and anchor were mixed together in 10% formamide and hybridized to the primary probes simultaneously. Subsequently, a click solution consisting of copper sulfate, BTTAA ligand, and sodium ascorbate was added to the sample and incubated at 37 °C for one hour, followed by exposure to UV light for five minutes. Next, the sample was washed with 65% formamide at 37 °C for one hour (the harsh wash). Finally, the readout probes were hybridized, and the sample was imaged. A schematic diagram providing an overview of these steps is shown in FIG. 13.

### Results:

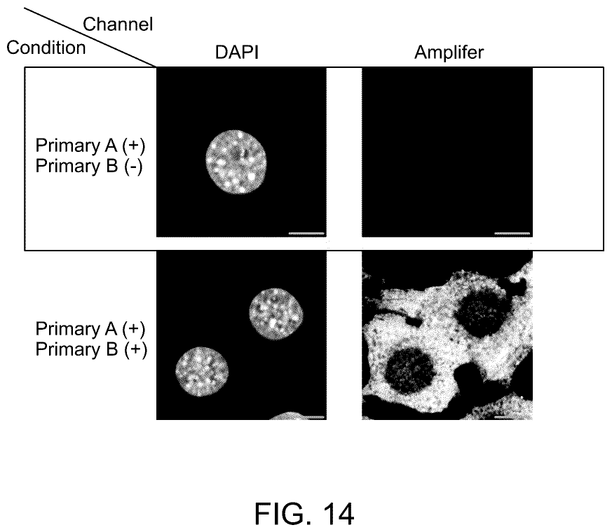

For the grab configuration, the positive condition included both Primary A and Primary B Grab probes; bright amplifier signals were observed after the harsh wash. In contrast, the negative condition included only the Primary A probe, and no amplifier signals were observed after the harsh wash (FIG. 14). These results suggest that a stable readout signal capable of withstanding the harsh wash requires the simultaneous presence of both Primary A and Primary B Grab probes, confirming the specificity of the coincidence detection design.

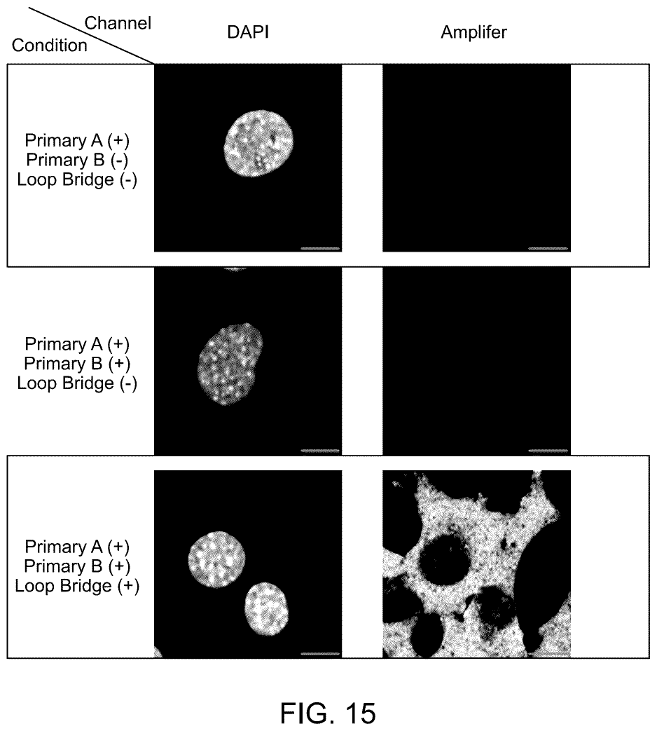

Similarly, for the loop configuration, the positive condition included Primary A, Primary B Loop, and Loop Bridge probes, and bright amplifier signals were observed after the harsh wash. Conversely, the negative condition included Primary A and Primary B Loop probes but lacked the Loop Bridge, and no signals were observed after the harsh wash (FIG. 15). This suggests the necessity of the Loop Bridge in this configuration to physically bridge the two primary probes, bringing them into the required proximity to enable the reaction and prevent signal loss during the washing step.

### Example 7

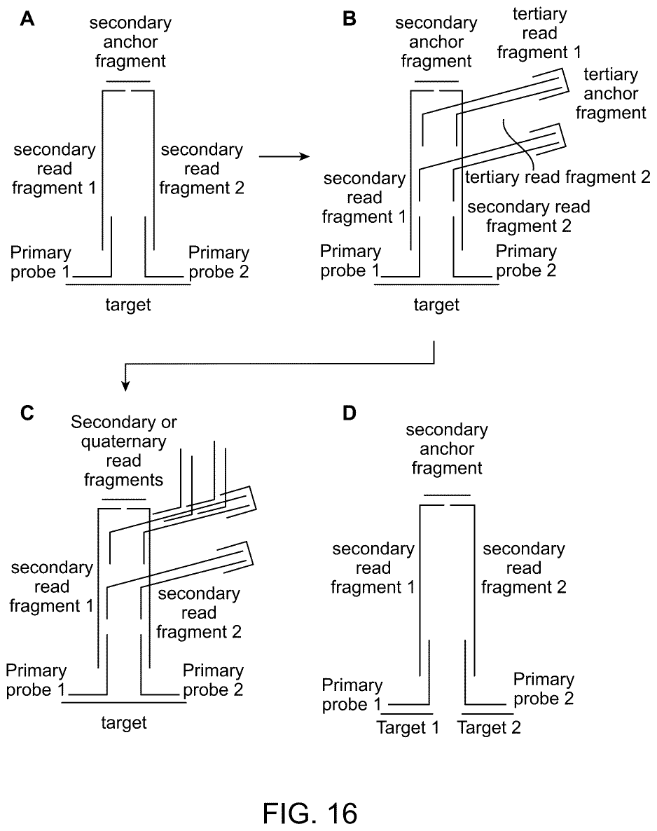

As shown in FIG. 16, an experiment was performed where 2 reading fragments were used (secondary 1 and 2). The anchoring fragment (bridge in the figure) was crosslinked to both reading fragments. The anchoring fragment was also crosslinked to the cell. The advantage of this was that even if the reading fragment “sticks” somewhere nonspecifically in the cell, it needs another reading fragment as well as an anchoring sequence for further amplification.

Further amplification was performed through tertiary reading fragments (1 and 2) with their own bridges, so that each step of amplification required three components. The chances that three components interacting nonspecifically was low compared to their interaction with specific targets. The anchoring fragment (bridge) contained additional groups to crosslink it to the cell. This allowed secondary reading fragments (secondary 1 and 2) to be crosslinked to the cell through their crosslink to the anchoring fragment (bridge), which allowed for harsh washes to remove any non-crosslinked secondary probes. Tertiary reading fragments were built upon the secondary read-anchor probes in the same way.

In another implementation, the secondary reading fragments, secondary anchoring fragments (bridge for secondary read fragments), tertiary reading fragments, and tertiary anchoring fragments (bridge for tertiary read fragments) were all mixed together to self-assemble in an “one-pot” reaction. The secondary anchoring fragment was designed to not bind to the two secondary reading fragments unless they are brought in proximity, in this case by the primary probes. The same is held with the tertiary anchoring fragment. Additionally, anchoring fragments were photo-crosslinked to the reading fragments (secondary and/or tertiary) by shining light on the sample periodically (or sporadically) to stabilize the amplified structure). Anchoring fragments in this “one-pot” amplification scheme may or may not need to be crosslinked to the cell, because the stabilization from binding to two secondary reading fragments was sufficiently energetically stable for self-assembly to occur.

### Example 8

### Method Description

In this implementation, to demonstrate FRET between amplified structures, pairs of probes were used that hybridized on adjacent regions of the RNA. Each unit consisted of two parts: the first part that hybridized to the target gene (the reverse complement of the target gene), fixing the unit in place, while the other part is the secondary amplifier binding sites that allowed different additional secondary amplifiers (single-strand DNA oligo) to bind to it. In this test, the spacer between the adjacent regions of the RNA can be 1-50 nt, and the binding sequences can be 25-100 nt each. The amplifier binding sites can be designed as 15-100 nt.

The secondary amplifiers have two tertiary binding sites, and the tertiary amplifiers have two secondary binding sites. Furthermore, tertiary amplifiers were used to bind to the secondary probes, thus enabling us to exponentially amplify and form a ball-like or tree-like amplified structure after sequentially hybridizing multiple rounds of secondary and tertiary amplifiers. Readout probes were then hybridized to the amplified structure, thereby amplifying the fluorescence signals of the target gene.

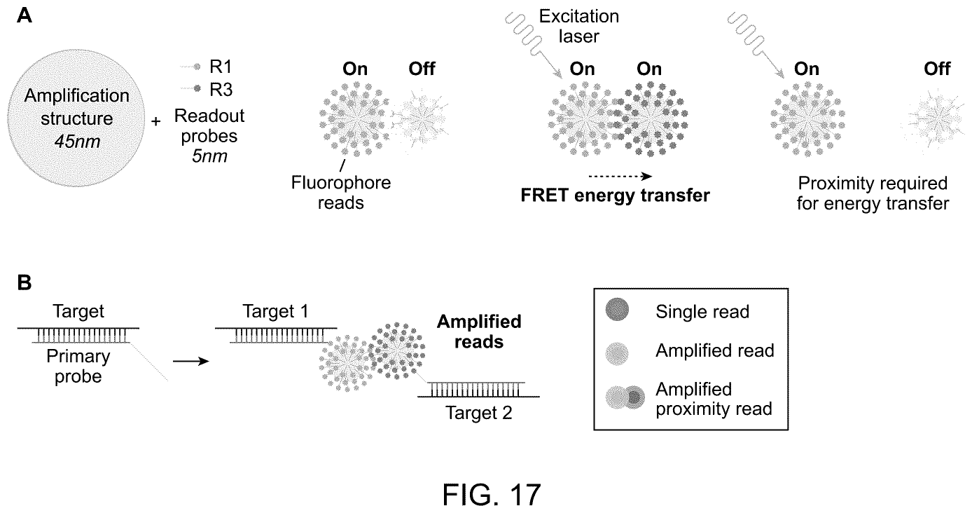

The scaffold of the complexes are ball-like structure with a diameter of roughly 45 nm after 12 rounds of exponential amplification, as shown in FIG. 17. All FRET pairs, or donor-acceptor pairs, could be used for detecting the interaction. Cy3B was chosen as the donor and Alexa647 as the acceptor specifically.

The readout probe were made by coupling fluorophores and the oligonucleotide via an NHS-amine reaction. Two kinds of readout probes were prepared for the experiment: one was attached to the donor (Cy3B), and the other is attached to the acceptor (Alexa 647). The donor readout probes hybridized/bound to the readout sites in the amplifier, R1, and the acceptor readout probes hybridized/bound to the readout sites in the amplifier, R3. Because the ball-like complexes of R1 and R3 were spatially close and interacted with each other (approximately 5 nm apart), this allowed FRET to occur between a fraction of the donors and acceptors on R1 and R3, which is shown in FIG. 17. This property was used to observe any FRET intensity in the FRET channel (using a donor excitation laser and reception of the acceptor emission) as an indication of whether there are interactions between the molecules of interest.

In order to accurately detect the interactions between amplifiers, it was crucial to accurately calculate the FRET intensity in the FRET channel. The raw intensity of each dot (emission) in the FRET channel not only contained the FRET intensity from the FRET phenomenon but also included donor leakage and acceptor leakage. Therefore, the donor leakage and acceptor leakage was subtracted from the raw intensity to obtain the true FRET intensity.

### Experiment Description:

The microscope used was a Dragonfly Confocal Microscope System, with a 63× oil objective. Three positions were imaged and captured as a 5-slice −Z-stack image, with a stepping size of 250 nm. Each slice and position was imaged using the 4 channels described above.

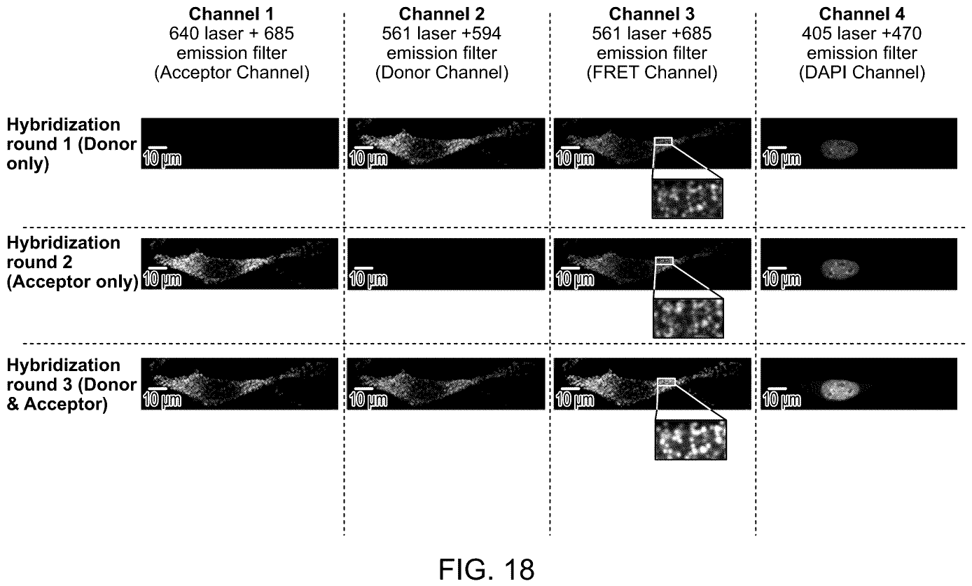

Three rounds of hybridization were performed in the following order: First, the donor was hybridized and then washed with a 65% formamide stripping buffer to strip off all of the donor from the R3 after imaging with four channels. In the second round, the acceptor was hybridized and then washed with a 65% formamide stripping buffer to strip off all of the acceptor from the R1 after imaging with four channels. In the third round, both the donor and acceptor were hybridized and then washed with a 65% formamide stripping buffer to strip off all of the acceptor from the R1 after imaging with four channels. Three positions were imaged for each hybridization round. The images for each hybridization round and channel are shown in FIG. 18, and the contrast settings for each individual channel are the same.

### Example 9

### Image Analysis

### Image Alignment:

The images were first aligned across each hybridization round using the DAPI image as a reference. Beads/fiducial markers were used on the coverslip as a reference to further align the images, ensuring that the drift of the same dots across each hybridization round was no larger than 1.5 pixels. Finally, each image was aligned across the four channels to eliminate chromatic aberration and drift.

### Dot Detection:

DAOStarFinder library in Python was used to detect all of the dots in all images, and the spatial information (x, y, z) and the intensity information (max intensity, flux, size) were recorded for further analysis.

### Donor/Acceptor Leakage and True FRET Intensity:

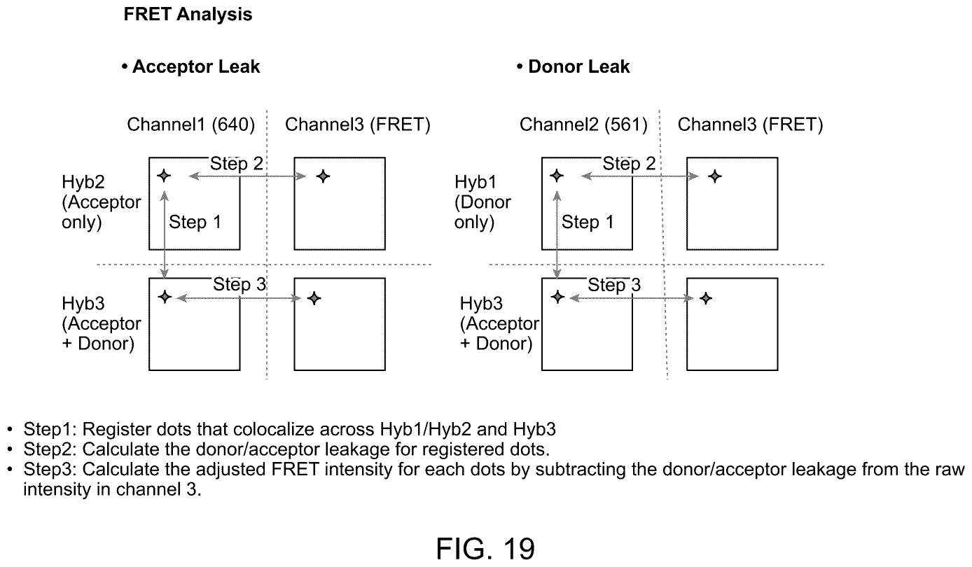

A simplified workflow is shown in FIG. 19. The donor and acceptor leakage were calculated in a similar way, and the analysis steps for calculating the donor leakage is described below.

In Step 1, all the dots that colocalize with each other were filtered and registered in Hybl Channel 2 and Hyb3 Channel 2. This step was performed first because if the “dots” do not appear in both Hybl and Hyb3, then it is very likely that they are random noise and do not require leakage calculation.

In Step 2, the “dots” were filtered that colocalize in Hybl Channel 2 and Hybl Channel 3 from the registered dots. Then, the leakage ratio of each “dot” was calculated by dividing the Channel 3 intensity by the Channel 2 intensity. The leakage ratio for each “dot” is not necessarily identical but can vary due to several factors, including the signal-to-noise ratio, differences in dye photo-physics, and variations in optical alignment across the imaging system. These factors introduce heterogeneity in the observed leakage ratios. Therefore, to obtain a robust estimation of the donor leakage, the leakage ratios of all dots were analyzed individually.

In Step 3, the “dots” were filtered that colocalize in Hyb3 Channel 2 and Hyb3 Channel 3 from the registered dots in Step 2. Then, the adjusted FRET intensity was calculated without donor leakage for each individual dot using the following formula: Channel 3 raw intensity −(donor leakage ratio for this dot×Channel 2 raw intensity).

The acceptor leakage is calculated in the same fashion, with some slight adjustments.

### For Acceptor Leakage:

In Step 1, instead of checking the colocalization in Hyb1 Channel 2 and Hyb3 Channel 2, the colocalization in Hyb2 Channel 1 and Hyb3 Channel 1 was checked.

In Step 2, “dots” were filtered that colocalize in Hyb2 Channel 1 and Hyb2 Channel 3 from the registered dots. Then, the leakage ratio of each dot was calculated by dividing the Channel 3 intensity by the Channel 1 intensity.

In Step 3, the “dots” were filtered that colocalize in Hyb3 Channel 1 and Hyb3 Channel 3 from the registered dots in Step 2. Then, the adjusted FRET intensity were calculated without acceptor leakage for each individual dot using the following formula:

Channel 3 raw intensity −(acceptor leakage ratio for this dot×Channel 1 raw intensity).

The true FRET intensity was then calculated by summing the two adjusted FRET intensities (adjusted FRET intensity without donor leakage and adjusted FRET intensity without acceptor leakage) and subtracting the raw intensity of the FRET channel. Furthermore, the FRET ratio was calculated by dividing the true FRET intensity by the raw intensity in the FRET channel. “Dots” with a positive FRET ratio indicate that there is an interaction between the two amplifiers, R1 and R3.

### Result:

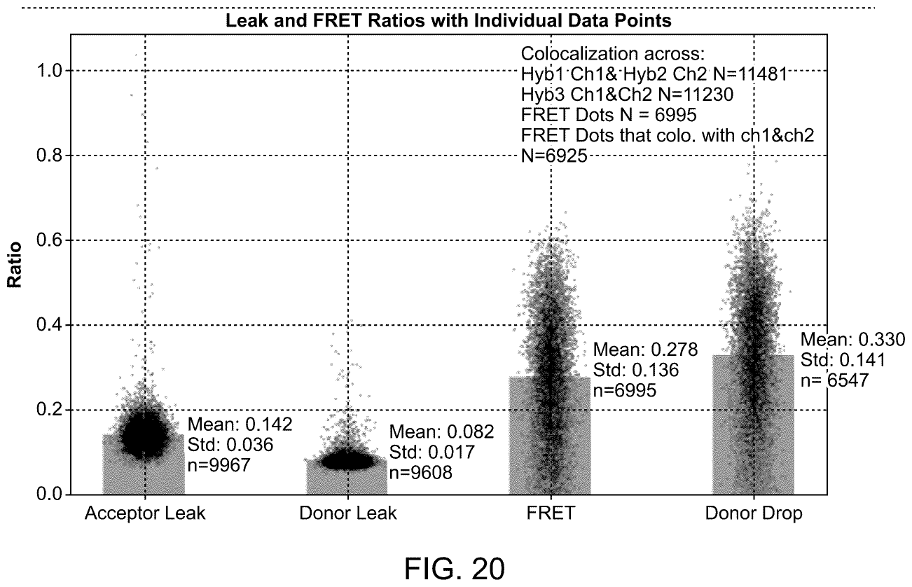

More than 10,000 dots in the same position were analyzed and shown in FIG. 20. The standard deviation and the mean of the donor and acceptor leak is primarily determined by the optical instrument features and FRET pairs used in the experiment. The standard deviation for the FRET intensity ratio is relatively large, but this is expected, since the distance between the donor and acceptor molecules undergoing FRET depends on their location on the ball-like structure. The donor acceptor pairs across two different balls are at variable relative distances, leading to non-uniform FRET efficiency. The large standard deviation reflects this variability. In contrast, the leakage signal from the donor or the acceptor follows a narrow distribution indicating a highly deterministic process.

In total, 6,995 dots were detected to have FRET between them, and 6,925 out of 6,995 of these dots colocalize with the dots that colocalize in Hyb2 Channel 1 and Hyb2 Channel 2, suggesting that the detection accuracy is almost 99%.

### REFERENCES

The following references were incorporated by their entirety.
Experimental design for kinetic analysis of protein-protein interactions with surface plasmon resonance biosensors, Karlsson, R. et al. Journal of Immunological methods. Volume 200, Issues 1-2, pages 121-133 (1997)
Transcriptome-scale super-resolved imaging in tissues by RNA seqFISH+, Eng, CH el al. Nature volume 568, pages 235-239 (2019)
International PCT Patent Application No. PCT/US2014/036258, file Apr. 30, 2014, and titled MULTIPLEX LABELING OF MOLECULES BY SEQUENTIAL HYBRIDIZATION BARCODING.
U.S. Provisional Application 63/741,406 filed on Jan. 2, 2025, READ-AND-ANCHOR SPLIT PROBE STRATEGY FOR HIGH-SPECIFICITY MOLECULAR PROFILING
U.S. Provisional Application No. 63/741,401, filed on Jan. 2, 2025, SEQFISH FRET HYBRIDIZATION.
U.S. Provisional Application No. 63/822,120, filed on Jun. 11, 2025, READ-AND-ANCHOR SPLIT PROBE STRATEGY FOR HIGH-SPECIFICITY MOLECULAR PROFILING.
Spatial Proofreading Amplification of in situ Transcript and Protein Signals, Carsten H. Tischbirek,* Katsuya L. Colón,* Saori Lobbia, Christopher J. Cronin, Long Cai.
International PCT Patent Application No. PCT/US2014/036258, filed Apr. 30, 2014, and titled MULTIPLEX LABELING OF MOLECULES BY SEQUENTIAL HYBRIDIZATION BARCODING.
International PCT Patent Application No. PCT/US2018/064616, filed Dec. 7, 2018, and titled MULTIPLEX LABELING OF MOLECULES Publication Classification BY SEQUENTIAL HYBRIDIZATION BARCODING WITH RAPID SWITCHING AND REHYBRIDIZATION OF PROBES.
International PCT Patent Application No. PCT/US2017/044994, filed Aug. 1, 2017, and titled SEQUENTIAL PROBING OF MOLECULAR TARGETS BASED ON PSEUDO—COLOR BARCODES WITH EMBEDDED ERROR CORRECTION MECHANISM.
International PCT Patent Application No. PCT/US2022/024494, filed Apr. 12, 2022, and titled HIGH-RESOLUTION WHOLE GENOME IMAGING BY NUCLEIC ACID LOCUS AND BLOCK CODING.
International PCT Patent Application No. PCT/US2022/032736, filed Jun. 8, 2022, and titled RATIOMETRIC SYMBOLS AND SEQUENTIAL CODING FOR MULTIPLEXED FISH.
International PCT Patent Application No. PCT/US2022/053995, filed Dec. 23, 2022, and titled SUPPRESSION OF NON-SPECIFIC SIGNALS BY EXONUCLEASES IN FISH EXPERIMENT.
International PCT Patent Application No. PCT/US2022/051737, filed Dec. 2, 2022, and titled METHOD OF MAPPING SPATIAL DISTRIBUTIONS OF CELLULAR COMPONENTS.
International PCT Patent Application No. PCT/US2022/017757, filed Feb. 24, 2022, and titled MULTIPLEXING OF EXPERIMENTAL CONDITIONS AND SAMPLES IN SPATIAL GENOMICS.
International PCT Patent Application No. PCT/US2025/015646, filed Feb. 12, 2025, and titled SUPER-RESOLVED OBJECT DETECTION WITH SPATIAL GENOMICS.

1.-69. (canceled)

70. A method for detecting one or more primary probes interacting with a substrate, comprising steps of:
(a) contacting the one or more primary probes with secondary reading fragments and secondary anchoring fragments, wherein each secondary reading fragment interacts with one of the primary probes and/or one of the secondary anchoring fragments, and wherein each secondary anchoring fragment interacts with one of the primary probes;
(b) linking secondary reading fragments and secondary anchoring fragments to form secondary probes;
(c) optionally, contacting secondary probes with tertiary reading fragments and tertiary anchoring fragments, wherein each tertiary reading fragment interacts with one of the secondary probes and/or one of the tertiary anchoring fragments, and wherein each tertiary anchoring fragment interacts with one of the secondary probes;
(d) optionally, linking tertiary reading fragments and tertiary anchoring fragments to form tertiary probes;
(e) optionally, linking secondary probes to primary probes at any of steps (b)-(d);
(f) optionally, linking tertiary probes to secondary probes and/or to the primary probes;
(g) optionally, washing the substrate to remove fragments and/or probes at any of steps (b)-(f);
(h) optionally, repeating any of steps (a)-(g);
(i) contacting primary, secondary, and/or tertiary probes with one or more readout probes; and
(j) detecting the one or more readout probes to detect the one or more primary probes interacting with the substrate.

71. The method of claim 70, further comprising:
(k) optionally, contacting the tertiary probes after any of steps (c)-(f) with quaternary reading fragments and quaternary anchoring fragments, wherein each quaternary reading fragment interacts with one of the tertiary probes and/or one of the quaternary anchoring fragments, and wherein each quaternary anchoring fragment interacts with one of the tertiary probes;
(l) optionally, linking quaternary reading fragments and quaternary anchoring fragments to form quaternary probes; and
(m) contacting the primary, secondary, tertiary, and/or quaternary probes with one or more readout probes.

72. The method of claim 71, wherein the steps (b)-(f) and/or (k)-(l) are performed concurrently with step (a).

73. A method for detecting one or more primary probes interacting with a substrate, comprising steps of:
(a) contacting the one or more primary probes with secondary reading fragments and optionally, secondary anchoring fragments, wherein each secondary reading fragment interacts with one of the primary probes and/or one of the secondary anchoring fragments, and wherein each secondary anchoring fragment interacts with one or more secondary reading fragments;
(b) optionally, linking the secondary reading fragments and secondary anchoring fragments to form secondary bridge complexes;
(c) optionally, contacting the secondary reading fragments with tertiary reading fragments and optionally, tertiary anchoring fragments, wherein each tertiary reading fragment interacts with one of the secondary reading fragments and/or one of the tertiary anchoring fragments, and wherein each tertiary anchoring fragment interacts with one or more tertiary reading fragments;
(d) optionally, linking tertiary reading fragments and tertiary anchoring fragments to form tertiary bridge complexes;
(e) optionally, linking secondary reading fragments to primary probes at any of steps (b)-(d);
(f) optionally, linking tertiary reading fragments to secondary reading fragments at any of steps (c)-(e);
(g) optionally, washing the substrate to remove fragments and/or probes at any of steps (b)-(f);
(h) optionally, repeating any of steps (a)-(g);
(i) contacting primary probes, secondary reading fragments, secondary anchoring fragments, tertiary reading fragments, and/or tertiary anchoring fragments with one or more readout probes; and
(j) detecting the one or more readout probes to detect the one or more primary probes interacting with the substrate.

74. The method of claim 73, further comprising:
(k) optionally, contacting the tertiary reading fragments during or after any of steps (a)-(f) with quaternary reading fragments and quaternary anchoring fragments, wherein each quaternary reading fragment interacts with one of the tertiary reading fragments and/or one of the quaternary anchoring fragments, and wherein each quaternary anchoring fragment interacts with one or more tertiary reading fragments;
(l) optionally, linking quaternary reading fragments and quaternary anchoring fragments to form quaternary bridge complexes; and
(m) contacting the primary probes, secondary reading fragments, tertiary reading fragments, quaternary reading fragments, secondary anchoring fragments, tertiary anchoring fragments, and/or quaternary anchoring fragments with one or more readout probes.

75. (canceled)

76. The method of claim 70, wherein the substrate is a cell or a hydrogel.

77. (canceled)

78. The method of claim 70, wherein the substrate comprises one or more molecular targets.

79. (canceled)

80. The method of claim 78, wherein the one or more molecular targets are selected from oligonucleotides, DNAs, DNA locus loci, RNAs, proteins, glycans, small molecules, and any combinations thereof.

81.-82. (canceled)

83. The method of claim 78, wherein the one or more primary probes interact with the one or more molecular targets.

84.-85. (canceled)

86. The method of claim 70, wherein the one or more primary probes are selected from oligonucleotides, DNAs, RNAs, small molecules, peptides, proteins, antibodies, antibodies conjugated to oligonucleotides, and combinations thereof.

87.-88. (canceled)

89. The method of claim 70, wherein the secondary reading fragments and/or secondary anchoring fragments hybridize to the one or more primary probes.

90. The method of claim 70, wherein the tertiary reading fragments and/or tertiary anchoring fragments hybridize to the secondary probes.

91. The method of claim 70, wherein the one or more readout probes hybridize to the primary, secondary, and/or tertiary probes.

92. The method of claim 70, wherein the substrate comprises one or more cellular components.

93. (canceled)

94. The method of claim 92, wherein the cellular components are selected from oligonucleotides, DNAs, RNAs, glycans, small molecules, peptides, proteins, antibodies, antibodies conjugated to oligonucleotides, organelles, subcellular compartments and combinations thereof.

95. (canceled)

96. The method of claim 71, further comprising linking the one or more primary probes, secondary reading fragments, secondary anchoring fragments, secondary probes, tertiary reading fragments, tertiary anchoring fragments, tertiary probes, quaternary reading fragments, quaternary anchoring fragments, and/or quaternary probes to the substrate at any of steps (a)-(f) or (k)-(l).

97. (canceled)

98. The method of claim 71, further comprising linking the one or more secondary reading fragments to the one or more secondary anchoring fragments, secondary probes, tertiary reading fragments, tertiary anchoring fragments, tertiary probes, quaternary reading fragments, quaternary anchoring fragments, and/or quaternary probes at any of steps (a)-(f) or (k)-(l).

99. The method of claim 71, further comprising linking the one or more secondary anchoring fragments to the secondary probes, the one or more tertiary reading fragments, tertiary anchoring fragments, tertiary probes, quaternary reading fragments, quaternary anchoring fragments, and/or quaternary probes at any of steps (a)-(f) or (k)-(l).

100.-103. (canceled)

104. The method of claim 70, wherein the one or more primary probes, secondary anchoring fragments, secondary reading fragments, tertiary reading fragments, tertiary anchoring fragments, quaternary reading fragments, and/or quaternary anchoring fragments comprise 5′ cross-linkable moieties, 3′ cross-linkable moieties, cross-linkable moieties between the 5′ and 3′ ends, or combinations thereof.

105. (canceled)

106. The method of claim 70, wherein the one or more readout probes comprise one or more detectable moieties.

107.-110. (canceled)

111. The method of claim 70, wherein the one or more readout probes are sequencing probes.

112.-147. (canceled)

148. A method, comprising steps of:
(a) contacting a cell comprising molecular targets with a plurality of primary probes, each primary probe interacting with a molecular target, so that the plurality comprises:
(i) a first primary probe that interacts with a first molecular target in the cell; and
(ii) a second primary probe that interacts with a second molecular target in the cell;
(b) forming, by contacting secondary probes to the plurality of primary probes, a first probe complex on the first primary probe and a second probe complex on the second primary probe;
(c) contacting a plurality of detectably labelled readout probes to binding sites on the first and second probe complexes, wherein the plurality detectably labelled readout probe comprises:
(i) a first readout probe that interacts with the first probe complex, wherein the first readout probe comprises a fluorescence donor;
(ii) a second readout probe that interacts with the second probe complex, wherein the second readout probe comprises a fluorescence acceptor; and
(f) imaging the cell so that the proximity between the first molecular target and the second molecular target is detected from the interaction of the fluorescent donor and fluorescent acceptor.

149.-152. (canceled)

153. A method, comprising steps of:
(a) contacting a cell comprising molecular targets with a plurality of primary probes, each primary probe interacting with a molecular target, so that the plurality comprises:
(i) a first primary probe that interacts with a first molecular target in the cell; and
(ii) a second primary probe that interacts with a second molecular target in the cell;
(b) contacting the primary probes with a plurality of secondary probes, wherein the plurality of secondary probes comprises:
(i) a first secondary probe that interacts with the first primary probe; and
(ii) a second secondary probe that interacts with the second primary probe;
(c) optionally, contacting the secondary probes with a plurality of tertiary probes, wherein the plurality of tertiary probes comprises:
(i) a first tertiary probe that interacts with the first secondary probe, and
(ii) a second tertiary probe that interacts with the second secondary probe;
(d) optionally, contacting the tertiary probes with a plurality of quaternary probes after, wherein the plurality of quaternary probes comprises:
(i) a first quaternary probe that interacts with the first tertiary probe or the first secondary probe; and
(ii) a second quaternary probe that interacts with the second tertiary probe or the second secondary probe,
(e) optionally, repeating any of the contacting steps (b), (c), or (d), wherein the secondary, tertiary, or quaternary probes comprise readout probe binding sites;
(f) contacting the secondary, tertiary, or quaternary probes with a plurality of readout probes, the plurality of readout probes comprises:
(i) a first readout probe that interacts with the first secondary probes, tertiary probes, or quaternary probes wherein the first readout probe comprises a moiety that is a fluorescent donor;
(ii) a second readout probe that interacts with the second secondary probes, tertiary probes, or quaternary probes wherein the second readout probe comprises a moiety that is a fluorescent acceptor; and
(g) imaging the cell so that the proximity between the first molecular target and the second molecular target is detected from the interaction of the fluorescent donor and fluorescent acceptor.

154.-177. (canceled)
